(19) United States
(12) Patent Application Publication
(10) Pub. No.: US 2026/0174822 A1
(43) Pub. Date: Jun. 25, 2026

(54) FUSION PROTEINS FOR THE TREATMENT OF NONALCOHOLIC STEATOHEPATITIS

(71) Applicant: Shattuck Labs, Inc., Austin, TX (US)

(72) Inventors: Taylor SCHREIBER, Austin, TX (US); George FROMM, Austin, TX (US)

(21) Appl. No.: 19/126,366

(22) PCT Filed: Nov. 2, 2023
(86) PCT No.: PCT/US2023/078517

§ 371 Date: May 1, 2025

Related U.S. Application Data
(60) Provisional application No. 63/382,085, filed on Nov. 2, 2022.
(60) Provisional application No. 63/386,073, filed on Dec. 5, 2022.
(60) Provisional application No. 63/579,233, filed on Aug. 28, 2023.

(51) Int. Cl. A61K 38/00

(52) U.S. Cl. CPC A61K 38/00

(57) ABSTRACT

The present disclosure relates to, inter alia, compositions and methods, including heterologous chimeric proteins that find use, inter alia, in the treatment of nonalcoholic fatty liver disease (NAFLD), non-alcoholic steatohepatitis (NASH), obesity, or metabolic syndrome.

### PRIORITY

This application claims the benefit of, and priority to, U.S. Provisional Application No. 63/382,085, filed Nov. 2, 2022, U.S. Provisional Application No. 63/386,073, filed Dec. 5, 2022, and U.S. Provisional Application No. 63/579,233, filed Aug. 28, 2023, the contents of each of which are hereby incorporated by reference in their entirety.

### TECHNICAL FIELD

The present disclosure relates to, inter alia, compositions and methods, including heterologous chimeric proteins that find use, inter alia, in the treatment of nonalcoholic fatty liver disease (NAFLD), non-alcoholic steatohepatitis (NASH), obesity, or metabolic syndrome.

### SEQUENCE LISTING

The instant application contains a sequence listing, which has been submitted in XML format via Patent Center. The contents of the XML copy named “SHK-075PC_116981-5075_Sequence_Listing”, which was created on Nov. 2, 2023, and is 118,048 bytes in size, are incorporated herein by reference in their entirety.

### BACKGROUND

Non-alcoholic fatty liver disease (NAFLD) causes symptoms ranging from steatosis to non-alcoholic steatohepatitis (NASH) and cirrhosis. NAFLD is the most common chronic liver disorder in western countries and the U.S., where about 30% of the adult population is believed to suffer from NAFLD. See, e.g., Bellentani et al., Epidemiology of non-alcoholic fatty liver disease, Dig Dis 28(1):155-61 (2010). NAFLD can progress to non-alcoholic steatohepatitis (NASH), which can lead to cirrhosis and hepatocellular carcinoma (HCC).

Currently there is no treatment approved for treating NAFLD or NASH, although weight loss through a combination of a healthy diet and exercise can help to some degree. Liver transplantation is performed to help some very serious NASH patients. Not surprisingly, patients with NASH have an overall higher mortality rate and the primary cause of death in the early stages of NASH is cardiovascular diseases, while the cause of death in patients with late-stage fibrosis is liver related. Thus, there is a large unmet medical need to identify and develop effective treatment options for the benefit of the patients.

### SUMMARY

In various aspects, the present disclosure provides compositions and methods that are useful, inter alia, in the treatment of nonalcoholic fatty liver disease (NAFLD), non-alcoholic steatohepatitis (NASH), obesity, or metabolic syndrome.

Accordingly, in aspects, the present disclosure provides a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In embodiments, the GLP-1 receptor agonist is selected from GLP-1, DPP4 degradation resistant GLP-1 (7-37, A8G), exenatide, lixisenatide, albiglutide, dulaglutide, or a variant thereof, having one or more amino acid mutations, independently selected from substitutions, insertions, deletions, and truncations. In embodiments, the GLP-1 receptor agonist has an amino acid sequence of any one of SEQ ID NOs: 57 to 62, 75, and 87-90, or a variant having about 1, 2, 3, 4, 5, 6, 7, 8, 9, or 10 or more amino acid mutations with respect to an amino acid sequence selected from SEQ ID NOs: 57 to 62, 75, and 87-90. In embodiments, the mutations are independently selected from substitutions, insertions, deletions, and truncations. In embodiments, GLP-1 receptor agonist is capable of binding a GLP-1 receptor. In embodiments, the GLP-1 receptor agonist is capable of stimulating insulin secretion and/or inhibiting glucagon secretion.

In embodiments, the fibroblast growth factor comprises FGF19, or an analog thereof. In embodiments, the analog of FGF19 is aldafermin (NGM282). In embodiments, the fibroblast growth factor is capable of activating FGFR4, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence of SEQ ID NOs: 65 or 66.

Additionally, or alternatively, in embodiments, the fibroblast growth factor comprises FGF21, or an analog thereof. In embodiments, the analog of FGF21 is selected from efruxifermin, LY2405319, FGF21 (RGE) and FGF21 (L146P). In embodiments, the fibroblast growth factor is capable of activating FGFR1c, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence selected from SEQ ID NOs: 66 to 68, 76 and 77.

In embodiments, the linker comprises a protease-cleavable polypeptide linker. In embodiments, the protease-cleavable polypeptide linker cleavable by a protease that is endogenous to a mammalian liver. In embodiments, the protease-cleavable linker is cleavable by a protease selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the protease-cleavable linker comprises a consensus recognition and/or cleavage site of a protease selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOS: 70 to 74.

In embodiments, the chimeric protein comprises two protease-cleavable polypeptide linkers. In embodiments, wherein the first protease-cleavable polypeptide linker is C terminal to the first domain and the second protease-cleavable polypeptide linker is N terminal to the second domain. In embodiments, the two protease-cleavable polypeptide linkers are cleavable by a protease that is endogenous to a mammalian liver. In embodiments, the two protease-cleavable polypeptide linkers are cleavable by a protease independently selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers comprise consensus recognition and/or cleavage sites of a proteases independently selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers are cleavable by a protease independently comprises an amino acid sequence selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

In embodiments, the first domain comprising a glucagon-like peptide-1 (GLP-1) receptor agonist and the protease-cleavable polypeptide linker is C terminal to the first domain; or the second domain comprises a glucagon-like peptide-1 (GLP-1) receptor agonist and the protease-cleavable polypeptide linker is N terminal to the second domain. In embodiments, the chimeric protein comprises two protease-cleavable polypeptide linkers, such protease-cleavable polypeptide linker independently selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74. In embodiments, the first protease-cleavable polypeptide linker is C terminal to the first domain and the second protease-cleavable polypeptide linker is N terminal to the second domain.

In embodiments, the linker comprises a hinge-CH2-CH3 Fc domain derived from IgG1. In embodiments, the IgG1 is human IgG1. In embodiments, the linker comprises a hinge-CH2-CH3 Fc domain derived from IgG4. In embodiments, the IgG4 is human IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 79. In embodiments, the linker further comprises the linker comprises one or more joining linkers, such joining linkers independently selected from SEQ ID NOs: 4 to 50 or 79. In embodiments, the linker comprises two or more joining linkers each joining linker independently selected from SEQ ID NOs: 4 to 50 or 79; wherein one joining linker is N terminal to the hinge-CH2-CH3-Fc domain and another joining linker is C terminal to the hinge-CH2-CH3-Fc domain.

In aspects, the present disclosure provides an isolated polynucleotide encoding the chimeric protein of any one of the embodiments disclosed herein.

In embodiments, the polynucleotide is mRNA or a modified mRNA (mmRNA). In embodiments, the polynucleotide is an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. In embodiments, the mmRNA further comprises a 5′-cap and/or a poly A tail.

In embodiments, the polynucleotide is DNA. In embodiments, the polynucleotide comprises a liver-specific control element. In embodiments, the liver-specific control element is a liver-specific promoter selected from albumin promoter, thyroxine-binding globulin (TBG) promoter, hybrid liver-specific promoter (HLP), human α1-antitrypsin promoter, LP1 promoter, and hemopexin promoter.

In aspects, the present disclosure provides a vector comprising the polynucleotide of any one of the embodiments disclosed herein.

In aspects, the present disclosure provides a host cell comprising the vector of any of the embodiments disclosed herein. A host cell comprising the mmRNA of any of the embodiments disclosed herein.

In aspects, the present disclosure provides a pharmaceutical composition comprising the chimeric protein of any one of the embodiments disclosed herein, the isolated polynucleotide of any one of the embodiments disclosed herein, or the vector of the embodiments disclosed herein, or the host of any of the embodiments disclosed herein, and a pharmaceutically acceptable carrier. In embodiments, the pharmaceutical composition comprises the mmRNA of any one of the embodiments disclosed herein.

In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticle (LNP), a lipoplex, or a liposome. In embodiments, the pharmaceutical composition is formulated as a lipid nanoparticle (LNP).

In embodiments, the pharmaceutical composition is formulated as a lipid nanoparticle (LNP). In embodiments, the LNP comprises a molar ratio of about 20-60% ionizable amino lipid, about 5-25% phospholipid, about 25-55% structural lipid, and about 0.5-1.5% PEG lipid. In embodiments, the LNP comprises a molar ratio of about 50% ionizable amino lipid, about 8-12% phospholipid, about 37-40% structural lipid, and about 1-2% PEG lipid. In embodiments, the lipid nanoparticles comprise lipids selected from one or more of an ionizable lipid (e.g., an ionizable cationic lipid selected from DLin-DMA, DLin-K-DMA, DLin-KC2-DMA, DLin-MC3-DMA, 98N12-5, a and C12-200); structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

In embodiments, the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the polynucleotide (e.g., mmRNA of any of the embodiments disclosed herein).

In embodiments, the lipid nanoparticles comprise lipids selected from one or more of an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the polynucleotide (e.g., mmRNA of any of the embodiments disclosed herein). In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g., C12, a PEG-dimyristyloxypropyl (C14), a PEG-dipalmityloxypropyl (C16), or a PEG-distearyloxypropyl (C18)).

In embodiments, the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In any of the embodiments disclosed herein, the pharmaceutical composition is formulated for parenteral administration. In any of the embodiments disclosed herein, the pharmaceutical composition is formulated for topical administration.

In aspects, the present disclosure provides a pharmaceutical composition comprising the mmRNA of any embodiment disclosed herein, or an LNP comprising an mmRNA of any embodiment disclosed herein. In embodiments, the pharmaceutical composition is formulated for parenteral administration.

In embodiments, the pharmaceutical composition comprises a modified mRNA (mmRNA) encoding a heterologous chimeric protein having an amino acid sequence that has at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence selected from SEQ ID NOs: 80-93. In embodiments, the pharmaceutical composition is formulated as an LNP comprising an ionizable amino lipid, a phospholipid, a structural lipid and a PEG lipid.

In embodiments, the pharmaceutical composition is formulated for parenteral administration. In embodiments, the pharmaceutical composition is formulated for intradermal, intramuscular, intraperitoneal, intraarticular, intravenous, subcutaneous, intraarterial or transdermal administration.

In aspects, the present disclosure provides a method of treating or preventing nonalcoholic fatty liver disease (NAFLD), non-alcoholic steatohepatitis (NASH), obesity, or metabolic syndrome, or of reducing blood glucose, or of reducing fed and fasting blood glucose, or of reducing steady state plasma insulin, or of reducing food intake, or of reducing serum cholesterol, or of reducing liver adiposity, or of reducing liver weight, or of reducing epididymal white adipose tissue (eWAT) accumulation, or of reducing subcutaneous white adipose tissue (sWAT) accumulation, or of decreasing hepatocellular ballooning, or of reducing liver fibrosis, or of reducing excessive fatty acid intake by liver cells, or of protecting liver cells from fatty acid-induced toxicity a subject in need thereof, the method comprising administering to the subject a chimeric protein of any one of the embodiments disclosed herein, the isolated polynucleotide of any one of the embodiments disclosed herein, or the vector of any of the embodiments disclosed herein, or the host cell of any of the embodiments disclosed herein, or the pharmaceutical composition of any one of the embodiments disclosed herein.

In aspects, the present disclosure provides a method of treating nonalcoholic fatty liver disease (NAFLD) or non-alcoholic steatohepatitis (NASH) in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

Any aspect or embodiment disclosed herein can be combined with any other aspect or embodiment as disclosed herein.

### BRIEF DESCRIPTION OF THE DRAWINGS

FIG. 1A and FIG. 1B show the non-limiting schematic illustrations of the chimeric proteins FGF19/FGF21-Fc-protease-cleavable linker-GLP-1 (FIG. 1A) and GLP-1-protease-cleavable linker-Fc-FGF19/FGF21 (FIG. 1B).

FIG. 2 shows the results of a Meso Scale Discovery (MSD) platform-based ELISA assay demonstrating the contemporaneous binding of the chimeric proteins disclosed herein to antibodies specific to FGF19 or FGF21 and detected using antibodies specific to the Fc domain.

FIG. 3A to FIG. 3D demonstrate the binding to human GLP-1 receptor by the purified GLP-1-RFRS-Fc-FGF19 or GLP-1-RFRS-Fc-FGF21 (RGE) chimeric proteins, and detected using an anti-FGF21 antibody (FIG. 3A and FIG. 3C) or the culture supernatant of cells transfected with modified mRNA (mmRNA) encoding the GLP-1-RFRS-Fc-FGF19 or GLP-1-RFRS-Fc-FGF21 (RGE) chimeric proteins and detected using an anti-FGF19 antibody (FIG. 3B and FIG. 3D) as detected using a Meso Scale Discovery (MSD) platform-based ELISA assay.

FIG. 4A and FIG. 4B demonstrate that the chimeric proteins disclosed herein bind to cells expressing GLP-1 and FGF receptors. FIG. 4A is line graph showing the binding of the GLP-1-RFRS-Fc-FGF21 (RGE) and GLP-1-RFRS-Fc-FGF19 chimeric proteins to CHO-K1 cells that are engineered to express a human GLP-1 receptor. FIG. 4B is a line graph showing the binding of the GLP-1-RFRS-Fc-FGF21 (RGE) and GLP-1-RFRS-Fc-FGF19 chimeric proteins to HepG2 cells, which express receptors for both FGF19 and FGF21.

FIG. 5 shows the results of a Meso Scale Discovery (MSD) platform-based ELISA assay demonstrating the contemporaneous binding to the indicated recombinant FGF receptor and recombinant GLP-1 receptor by the chimeric proteins produced by cells transfected with modified mRNA (mmRNA) encoding the GLP-1-RFRS-Fc-FGF19 or GLP-1-RFRS-Fc-FGF21 (RGE) chimeric proteins. Cells expressing eGFP were used as negative control. Dotted lines show the amount of background signal generated by the culture supernatants of the cells expressing eGFP.

FIG. 6 shows the results of Meso Scale Discovery (MSD) platform-based ELISA assay demonstrating the contemporaneous binding to recombinant mouse GLP-1 receptor and Klotho alone or in the combination with the indicated recombinant FGF receptor by the chimeric proteins produced from cells transfected with modified mRNA (mmRNA) encoding the GLP-1-Fc-FGF19, GLP-1-RFRS-Fc-FGF19, or GLP-1-RFRS-Fc-FGF21 (RGE) chimeric proteins chimeric proteins. Supernatants from cells expressing eGFP were used as negative control.

FIG. 7A shows the schematic representation of the GLP-1R reporter assay. FIG. 7B demonstrates the activation of GLP-1R receptor by the purified chimeric proteins using the experimental system of FIG. 7A. FIG. 7C demonstrate the activation of GLP-1R receptor by culture supernatants of cells transfected with modified mRNA encoding the chimeric proteins using the experimental system of FIG. 7A. FIG. 7D demonstrates the activation of GLP-1R receptor using a second cell-based reporter assay by the culture supernatants of cells transfected with modified mRNA encoding the chimeric proteins based on the detection of CAMP in CHO-K1 cells expressing human GLP-1 receptor.

FIG. 8A shows the schematic representation of the cell based reporter assay specific for activation of FGFR1/Klotho receptor. FIG. 8B demonstrate the activation of FGFR1/Klotho by the purified chimeric proteins. FIG. 8C demonstrate the activation of FGFR1/Klotho by the culture supernatants of cells transfected with modified mRNA encoding the GLP-1-Fc-FGF19, or GLP-1-RFRS-Fc-FGF21 (RGE) chimeric proteins relative to un-transfected cell supernatants as a negative control.

FIG. 9A and FIG. 9B demonstrate the activation of GLP-1R and glucose-stimulated insulin secretion (GSIS) induced by the chimeric proteins disclosed herein. FIG. 9A demonstrates the activation of GLP-1R by the GLP-1-Fc fusion protein, the GLP-1-Fc-FGF19 or GLP-1-Fc-FGF21 chimeric proteins, or dulaglutide as a positive control. The reporter assay used in this figure is identical to the CHO-K1/hGLP1R CAMP assay used in FIG. 7D. FIG. 9B demonstrates the glucose-stimulated insulin secretion (GSIS) by the rat insulinoma INS-1 cells harboring a cAMP-luciferase reporter gene upon the treatment with dulaglutide, tirzepatide, or the GLP-1-Fc-FGF19 chimeric protein.

FIG. 10A to FIG. 10E demonstrate the in vivo activity of the GLP-1-Fc-FGF21 fusion protein and the mmRNA encoding the GLP-1-Fc-FGF21 fusion protein in a mouse model of obesity, hepatic steatosis and early-stage liver fibrosis. FIG. 10A shows the schematic representation of generation of a mouse model used in this study. Shown are results demonstrating the relative effects on body weight (FIG. 10B), food intake (FIG. 10C), plasma insulin and triglycerides (FIG. 10D), and liver lipin1 and Glut4 mRNA (FIG. 10E) in mice treated with dulaglutide as a positive control, a human GLP1-Fc-FGF21 recombinant fusion protein, or mice treated with an mmRNA/LNP encoding human GLP1-Fc-FGF21.

FIG. 11A to FIG. 11L demonstrate the efficacy of the GLP-1-Fc-FGF19 and GLP-1-Fc-FGF21 (RGE) chimeric proteins in a mouse model of obesity, hepatic steatosis and early-stage liver fibrosis. Shown are body weight (FIG. 11A), plasma glucose level (FIG. 11B), food consumption (FIG. 11C), liver weight (FIG. 11D), epididymal white adipose tissue (eWAT) weight (FIG. 11E) and subcutaneous white adipose tissue (sWAT) weight (FIG. 11F) in mice treated with tirzepatide as a positive control, or with a recombinant human GLP1-Fc-FGF19 fusion protein, or with an mmRNA/LNP encoding human GLP1-Fc-FGF19. Liver histology was performed to determine the degree of hepatic steatosis in mice treated with the indicated recombinant proteins or mmRNA/LNP encoding the indicated recombinant protein (FIG. 11G stained with H&E and FIG. 11H stained with picrosirius red). RNA was also isolated from the liver of treated mice to determine the expression of various inflammatory markers including glut4 mRNA expression (FIG. 11I) or lipin1 mRNA expression (FIG. 11J). Finally, plasma insulin (FIG. 11K), and serum cholesterol (FIG. 11L) was evaluated from mice treated as indicated in the mouse model of FIG. 10A.

FIG. 12A demonstrates that the GLP-1-Fc-FGF21 (RGE) chimeric protein or the mmRNA encoding the GLP-1-Fc-FGF21 (RGE) fusion protein decrease lipid intake in HEPG2 cells. FIG. 12B demonstrates that the GLP-1-Fc-FGF21 (RGE) chimeric protein or the mmRNA encoding the GLP-1-Fc-FGF21 (RGE) fusion protein decrease lipid intake-associated apoptosis of HEPG2 cells. FIG. 12C demonstrates that the GLP-1-Fc-FGF19 chimeric protein or the mmRNA encoding the GLP-1-Fc-FGF19 fusion protein decrease lipid intake in HEPG2 cells. FIG. 12D demonstrates that the GLP-1-Fc-FGF19 chimeric protein or the mmRNA encoding the GLP-1-Fc-FGF19 fusion protein decrease lipid intake-associated apoptosis of HEPG2 cells.

### DETAILED DESCRIPTION

The present disclosure is based, in part, on the creation of a fusion protein comprising a glucagon-like peptide-1 (GLP-1) receptor agonist, which is connected via a to a protease-cleavable linker to a polypeptide comprising a hinge-CH2-CH3 Fc domain-fibroblast growth factor 19 (FGF19), FGF21, FGF23, or a variant thereof. Surprisingly, the fusion proteins disclosed herein bind to the GLP-1 receptor with an EC₅₀ that is orders of magnitude lower compared to the recombinant purified GLP-1 protein, indicating more potent binding. The present disclosure is based, in part, on the delivery of a nucleic acid encoding the fusion protein to liver, expression of the fusion protein in liver, cleavage of the protease-cleavable linker in the liver, leading to the release of the glucagon-like peptide-1 (GLP-1) receptor agonist in the circulation. As shown herein, the modified mRNA mmRNA disclosed herein drives the expression of the disclosed fusion protein that contemporaneously binds to the GLP-1 receptor and the FGF receptor.

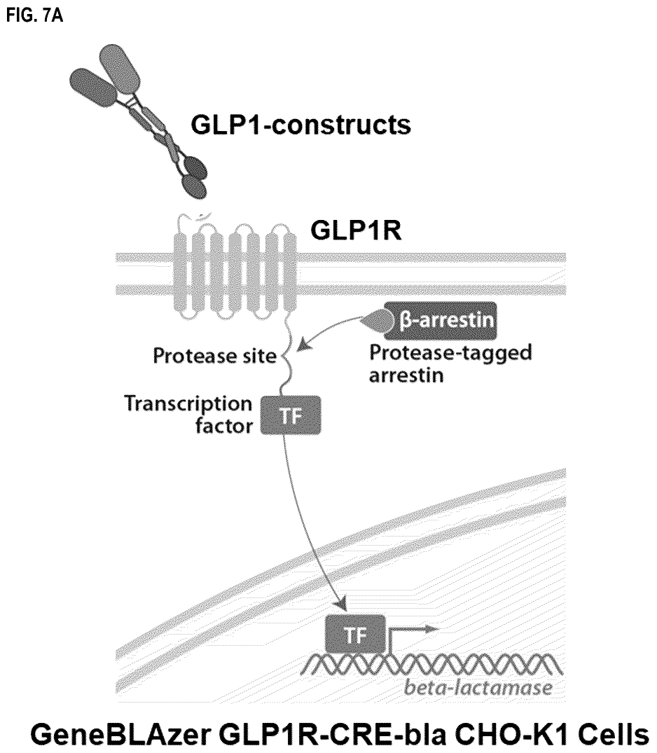

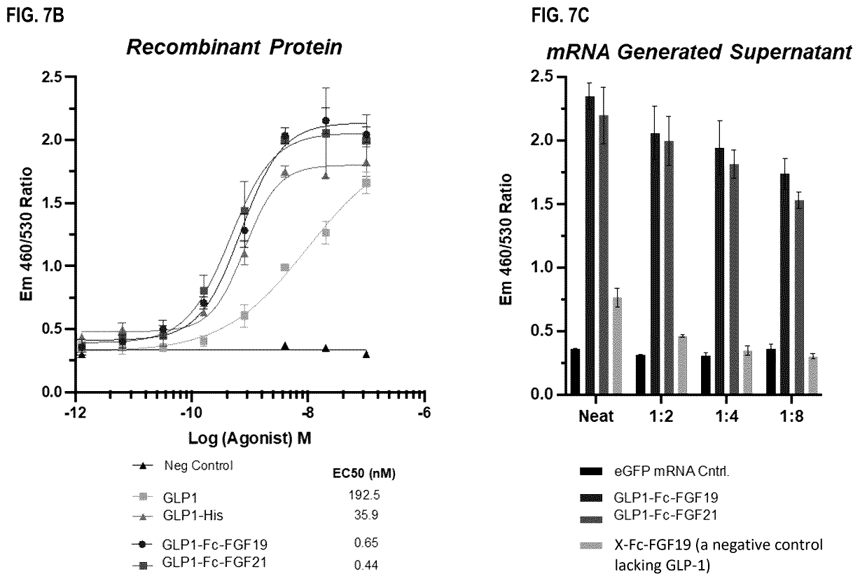

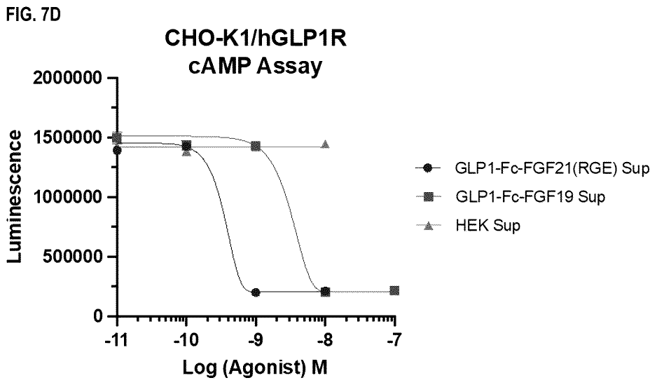

The present disclosure is based, in part, on the observation that the chimeric proteins disclosed herein and the nucleic acids encoding the chimeric proteins disclosed herein (without limitation, e.g., mmRNA) activate the GLP-1R receptor with a higher potency compared to the recombinant purified GLP-1-His, GLP-1-RFRS-His proteins lacking the FGF domain (FIG. 7B).

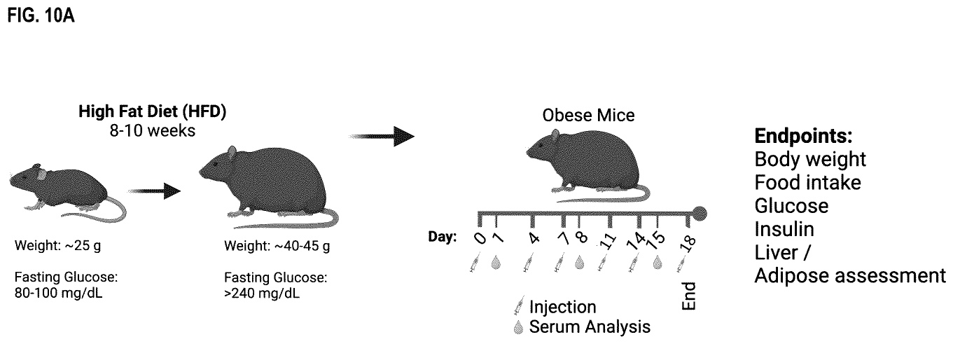

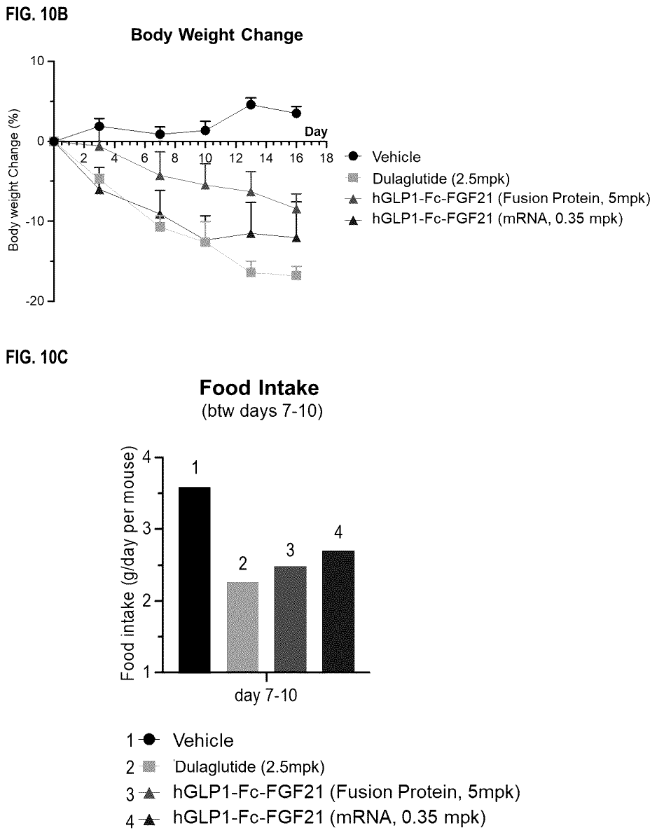

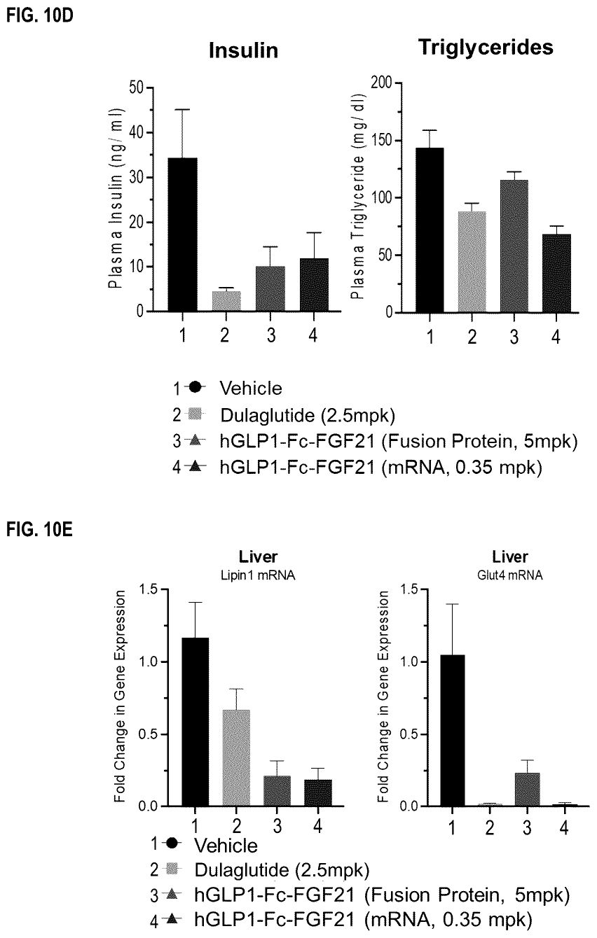

The present disclosure is based, in part, on the observation that the chimeric proteins disclosed herein and the nucleic acids encoding the chimeric proteins disclosed herein (without limitation, e.g., mmRNA) control body weight, plasma insulin, blood glucose, serum cholesterol, food intake, insulin resistance, obesity, Type II diabetes, metabolic syndrome, hyperglycemia, liver adiposity, liver weight, epididymal white adipose tissue (eWAT) accumulation, subcutaneous white adipose tissue (sWAT) accumulation, excessive fatty acid intake by liver cells, fatty acid toxicity to liver, hepatocellular ballooning liver, steatosis, and fibrosis. Surprisingly, as shown herein, the chimeric proteins disclosed herein and the mmRNA encoding the chimeric proteins disclosed herein are more effective in the downregulation of lipin1 compared to dulaglutide (FIG. 10E). Therefore, it is expected that the chimeric proteins disclosed herein and the mmRNA encoding the chimeric proteins disclosed herein are effective against hyperglycemia and insulin resistance. See Ryu et al., TORC2 Regulates Hepatic Insulin Signaling via a Mammalian Phosphatidic Acid Phosphatase, LIPIN1, Cell Metabolism 9:240-251 (2009).

Accordingly, In aspects, the present disclosure provides a chimeric protein and an isolated nucleic acid encoding the chimeric protein, wherein the chimeric protein has a general structure of: N terminus—(a)-(b) (c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist. In embodiments, the chimeric protein binds the GLP-1 receptor more potently compared to GLP-1 itself.

### Non-Alcoholic Fatty Liver Disease (NAFLD)

Non-alcoholic fatty liver disease (NAFLD) has a broad spectrum of pathologies, ranging from simple steatosis to non-alcoholic steatohepatitis (NASH), with potential progression to cirrhosis and hepatocellular carcinoma (HCC). NAFLD is caused by a combination of genetic and environmental factors and sedentary lifestyle. The metabolic syndrome, obesity, insulin resistance (IR), and type 2 diabetes are believed to manifest in NAFLD. The global prevalence of NAFLD is around 25% in adults, including the high prevalence in South America (31%) and the Middle East (32%), followed by Asia (27%), North America (24%), and Europe (23%). Cano et al., Role of Endocrine-Disrupting Chemicals in the Pathogenesis of Non-Alcoholic Fatty Liver Disease: A Comprehensive Review, Int J Mol Sci. 22(9): 4807 (2021). No treatment has been approved for NAFLD, and current treatments target the causes by reducing body weight (BW) and exercise. Untreated NAFLD can progress to NASH, to cirrhosis and hepatocellular carcinoma (HCC).

### Non-Alcoholic Steatohepatitis (NASH)

Nonalcoholic Steatohepatitis (NASH) is characterized, without limitation, by the presence of steatosis, lobular inflammation, cellular ballooning and varying degrees of fibrosis. It is a common, often “silent” disease. While the symptoms resemble alcoholic liver disease, but it occurs in people who drink little or no alcohol.

Most people with NASH are asymptomatic. Nevertheless, NASH can be severe and can lead to cirrhosis, in which the liver is permanently damaged and scarred and no longer able to work properly. Cirrhosis can progress even further to become hepatocellular carcinoma. Approximately 10-15% of patients with histologically proven NASH progress to cirrhosis and its sequelae such as liver failure and hepatocellular carcinoma (HCC).

NASH affects 2 to 5% of Americans. An additional 10 to 20 percent of Americans have fat in their liver, but no inflammation or liver damage, a condition called “fatty liver” (or steatosis). Although having fat in the liver is not normal, by itself it probably causes little harm or permanent damage.

NASH is usually first suspected in patients showing abnormal in liver function tests, such as alanine aminotransferase (ALT) or aspartate aminotransferase (AST) and triglycerides (TG). If further evaluation shows no apparent reason for liver disease (e.g., viral hepatitis, or excessive alcohol consumption), imaging studies are performed, which can show fat in liver. Liver biopsy (e.g., needle biopsy) is then used for definitive diagnosis of NASH. NASH is diagnosed based on the presence of steatosis (fatty liver), inflammation, and fibrosis.

Patients generally feel well in the early stages and only begin to have symptoms, such as fatigue, weight loss, and weakness, once the disease is more advanced or cirrhosis develops. The progression of NASH can take years, even decades. NASH can slowly worsen, causing increased fibrosis, which can lead to cirrhosis. Liver transplantation is the only treatment for advanced cirrhosis with liver failure, and transplantation is increasingly performed in people with NASH.

Overweight or obese patients with elevated blood lipids, such as cholesterol and triglycerides, and diabetes or pre-diabetes are at risk of NASH. Obesity, increased BMI and Type 2 Diabetes are associated with an increased risk of NASH. It is also well established that genetic factors predispose individuals to NAFLD and around 25% of people diagnosed with NAFLD have polymorphisms in adiponutrin (PNPLA3). However, some NASH patients are not obese, do not have diabetes or pre-diabetes, and have normal blood cholesterol and lipids. NASH can occur without any apparent risk factor and can even occur in children. Thus, NASH is not simply obesity that affects the liver.

While the underlying mechanisms for the liver injury that causes NASH is not known, several factors are possible candidates. These include insulin resistance, release of toxic inflammatory proteins by fat cells (cytokines) and oxidative stress. No specific therapies for NASH exist. The most important recommendations given to persons with this disease are to reduce their weight (if obese or overweight), follow a balanced and healthy diet, increase physical activity, avoid alcohol, avoid unnecessary medications and control their blood sugar, usually by using diabetes medications.

### Fusion Proteins of the Present Disclosure

Accordingly, In aspects, the present disclosure provides a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist. In embodiments, the chimeric protein is administered to a patient. In embodiments, a nucleic acid encoding the chimeric protein (e.g. modified mRNA or DNA) is administered to a patient. In embodiments, the nucleic acid encoding harbors control elements that enable the expression of the chimeric protein (e.g. modified mRNA or DNA) is the liver.

### GLP-1 Receptor Agonists

Glucagon-like peptide 1 (GLP-1) is a 30-amino acid peptide hormone produced in the intestine. GLP-1 is normally produced after meals and stimulates insulin secretion and inhibits glucagon secretion. It is also involved in the regulation of B-cell growth and survival, gastric emptying, and appetite. In the body, GLP-1 is degraded by dipeptidyl peptidase IV and has a short half-life of around 2 minutes. Reduced GLP-1 secretion is associated with type 2 diabetes and the development of obesity-two of the conditions associated with increased risk of NAFLD and NASH.

GLP-1R is coupled to a G protein that, once activated, increases intracellular cyclic AMP (CAMP) and induces downstream signaling such as the activation of protein kinase A (PKA), extracellular signal-regulated kinase (ERK)1/2 and phosphoinositol 3 kinase (PI3K)/protein kinase B (PKB). See, e.g., Andreozzi et al., The GLP-1 receptor agonists exenatide and liraglutide activate Glucose transport by an AMPK-dependent mechanism, J Transl Med. 14:229 (2016).

In embodiments, the chimeric proteins disclosed herein have a general structure of: N terminus—(a)-(b)-(c)—C terminus, wherein (a) is a first domain comprising a glucagon-like peptide-1 (GLP-1) receptor agonist. In these embodiments, (b) is a linker adjoining the first domain and a second domain, optionally wherein the linker comprises one or more protease-cleavable polypeptide linkers, and/or a hinge-CH2-CH3 Fc domain. In these embodiments, (c) is the second domain comprising a fibroblast growth factor 19 (FGF19), FGF21 or a variant thereof, or an analog thereof.

In alternative embodiments, the chimeric proteins disclosed herein have a general structure of: N terminus-(a)-(b)-(c)—C terminus, wherein (c) is a first domain comprising a glucagon-like peptide-1 (GLP-1) receptor agonist. In these embodiments, (b) is a linker adjoining the first domain and a second domain, optionally wherein the linker comprises one or more protease-cleavable polypeptide linkers, and/or a hinge-CH2-CH3 Fc domain. In these embodiments, (a) is the second domain comprising a fibroblast growth factor 19 (FGF19), FGF21 or a variant thereof, or an analog thereof.

In embodiments, the GLP-1 receptor agonist signals through its receptor, GLP-1 receptor (GLP-1R), a G-protein coupled receptor. In embodiments, the GLP-1 receptor agonist activates the GLP-1R on the surface of pancreatic B-cells. In embodiments, the GLP-1 receptor agonist induces increased insulin production by the pancreatic B-cells in a glucose dependent manner in response to the GLP-1R activation. In embodiments, the GLP-1 receptor agonist activates the GLP-1R on the surface of pancreatic q-cells. In embodiments, the GLP-1 receptor agonist activates GLP-1R suppresses glucose-dependent glucagon secretion by the pancreatic q-cells in response to the GLP-1R activation.

In embodiments, any of a number of drugs that mimic the action of GLP-1 by binding and activating the GLP-1 receptor is suitable. In embodiments, the GLP-1 receptor agonist is a short acting form (without limitation, e.g., exenatide). In embodiments, the GLP-1 receptor agonist is a long acting forms (without limitation, e.g., dulaglutide and liraglutide).

In embodiments, the GLP-1 receptor agonist is wild-type human GLP-1. In embodiments, the GLP-1 receptor agonist is GLP-1 receptor agonists. In embodiments, the GLP-1 receptor agonist is variant of these peptides that can activate the GLP-1 receptor. Suitable GLP-1 receptor agonists are disclosed in U.S. Pat. Nos. 5,188,666, 5,120,712, 5,523,549, 5,512,549, 5,977,071, 6,191,102; 6,956,026; 6,506,724; 6,703,359; 6,858,576; 6,872,700; 6,902,744; 7,157,555; 7,223,725; 7,220,721; 9,161,953; PCT International Publication Nos: WO 1998/008871; WO 1998/05351; WO 1999/07404; WO 1999/25727; WO 1999/25728; WO 1999/40788; WO 2000/034331; WO 2000/41546; WO 2000/41548; WO 2000/069911; WO 2000/73331; WO 2001/004156; WO 2001/51078; WO 2003/018516; WO 2003/099314; U.S. Publication No. 2003/0036504; and U.S. Publication No. 2006/0094652, the entire contents of which are hereby incorporated by reference in their entirety.

GLP-1 is produced by the alpha cells of the pancreas and in the intestinal L cells in the distal ileum and colon in form of a precursor called preglucagon that is cleaved in different organs into glicentin, glicentin-related pancreatic polypeptide (GRPP), oxyntomodulin, glucagon, glucagon-like peptide 1 (GLP-1, indicated in a boldface-underlined font), and glucagon-like peptide 2 (GLP-2). Preglucagon has the following sequence (: MKSIYFVAGLFVMVQGSWQRSLQDTEEKSRSFSASQADPLSDPDQMNEDKRHSQGTFTSDYSKYLDSR RAQDFVQWLMNTKRNRNNIAKRRHDEFERHAEGTFTSDVSSYLEGQAAKEFIAWLVKGRGRRDFPEEVAI VEELGRRHADGSFSDEMNTILDNLAARDFINWLIQTKITDRK (SEQ ID NO: 87)

In embodiments, the GLP-1 receptor agonist is GLP-1 having the following sequence:

(SEQ ID NO: 57)
HAEGTFTSDVSSYLEGQAAKEFIAWLVKGR

In embodiments, the GLP-1 receptor agonist is a GLP-1, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 57.

In embodiments, the GLP-1 receptor agonist is DPP4 degradation resistant GLP-1 (7-37, A8G) having the following sequence:

(SEQ ID NO: 75)
HGEGTFTSDVSSYLEEQAAKEFIAWLVKGRG

In embodiments, the GLP-1 receptor agonist is a GLP-1, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 75.

In embodiments, the GLP-1 receptor agonist is exenatide having the following sequence:

(SEQ ID NO: 58)
HGEGTFTSDLSKQMEEEAVRLFIEWLKNGGPSSGAPPPS

In embodiments, the GLP-1 receptor agonist is an exenatide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 58.

In embodiments, the GLP-1 receptor agonist is GLP-1 having the following sequence, and is also referred to herein as GLP-1 (1-36):

(SEQ ID NO: 88)
HDEFERHAEGTFTSDVSSYLEGQAAKEFIAWLVKGR

In embodiments, the GLP-1 receptor agonist is a GLP-1, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 88.

In embodiments, the GLP-1 receptor agonist is GLP-1 having the following sequence, and is also referred to herein as GLP-1 (7-36) or GLP-1:

(SEQ ID NO: 89)
HAEGTFTSDVSSYLEGQAAKEFIAWLVKGR

In embodiments, the GLP-1 receptor agonist is a GLP-1, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 89.

In embodiments, the GLP-1 receptor agonist lixisenatide is having the following sequence:

(SEQ ID NO: 59)
HGEGTFTSDLSKQMEEEAVRLFIEWLKNGGPSSGAPPSKKKKKK

In embodiments, the GLP-1 receptor agonist is a lixisenatide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 59.

In embodiments, the GLP-1 receptor agonist is albiglutide having the following sequence:

(SEQ ID NO: 60)
HGEGTFTSDVSSYLEGQAAKEFIAWLVKGR

In embodiments, the GLP-1 receptor agonist is an albiglutide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 60.

In embodiments, the GLP-1 receptor agonist is liraglutide having the following sequence:

(SEQ ID NO: 90)
HAEGTFTSDVSSYLEGQAAKEFIAWLVRGRG

In embodiments, the GLP-1 receptor agonist is an liraglutide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 90.

In embodiments, the GLP-1 receptor agonist is exendin-4 having the following sequence:

(SEQ ID NO: 61)
HGEGTFTSDLSKQMEEEAVRLFEWLKNGGPSSGAPPPS

In embodiments, the GLP-1 receptor agonist is an exendin-4, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 61.

In embodiments, the GLP-1 receptor agonist is the GLP-1 moiety of dulaglutide having the following sequence:

(SEQ ID NO: 62)
HGEGTFTSDVSSYLEEQAAKEFIAWLVKGGG_

In embodiments, the GLP-1 receptor agonist is the GLP-1 moiety of dulaglutide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 62.

Dulaglutide (GLP-1 moiety-Fc fusion protein; GLP-1 moiety underlined, (GGGGS)₃ shown in a boldface font) has the following sequence:

(SEQ ID NO: 63)
HGEGTFTSDVSSYLEEQAAKEFIAWLVKGGGGGGGGGGGGSGGGGSAES
KYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSR
WQEGNVFSCSVMHEALHNHYTQKSLSLSLG

In embodiments, the GLP-1 receptor agonist is an GLP-1 moiety of dulaglutide, or a variant thereof having one or more amino acid mutations, independently selected from substitutions, insertions, deletions, and truncations. In embodiments, the GLP-1 receptor agonist has an amino acid sequence having about 1, 2, 3, 4, 5, 6, 7, 8, 9, or 10 or more amino acid mutations with respect to an amino acid sequence selected from SEQ ID NO: 62.

In embodiments, the GLP-1 receptor agonist is an GLP-1 moiety of liraglutide, or an amino acid sequence having about 1, 2, 3, 4, 5, 6, 7, 8, 9, or 10 or more amino acid mutations with respect to an amino acid sequence of the GLP-1 moiety of liraglutide. In embodiments, the GLP-1 receptor agonist is an GLP-1 moiety of semaglutide, or an amino acid sequence having about 1, 2, 3, 4, 5, 6, 7, 8, 9, or 10 or more amino acid mutations with respect to an amino acid sequence of the GLP-1 moiety of semaglutide. In embodiments, the GLP-1 receptor agonist is an GLP-1 moiety of taspoglutide, or an amino acid sequence having about 1, 2, 3, 4, 5, 6, 7, 8, 9, or 10 or more amino acid mutations with respect to an amino acid sequence of the GLP-1 moiety of taspoglutide.

In embodiments, the chimeric protein of the disclosure binds to human GLP-1 receptor with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human GLP-1 receptor with a Ko of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human GLP-1 receptor with a K_{D} of from about 300 pM to about 700 pM.

GLP-1 derivatives can be constructed from available structural data, including that described by Chang et al., Structure and Folding of Glucagon-like Peptide-1-(7-36)-amide in Trifluoroethanol Studied by NMR, Magn Reson Chem 39:477-483 (2001); Underwood et al., Crystal structure of glucagon-like peptide-1 in complex with the extracellular domain of the glucagon-like peptide-1 receptor, J Biol Chem 285:723-730 (2010); Lau et al., Discovery of the Once-Weekly Glucagon-Like Peptide-1 (GLP-1) Analogue Semaglutide, J Med Chem 58:7370-7380 (2015); Oddo et al., alpha-Helix or beta-Turn? An Investigation into N-Terminally Constrained Analogues of Glucagon-like Peptide 1 (GLP-1) and Exendin-4, Biochemistry 57:4148-4154 (2018); Zhang et al., Cryo-EM structure of the activated GLP-1 receptor in complex with a G protein, Nature 546:248-253 (2017); Bueno et al., Structural insights into probe-dependent positive allosterism of the GLP-1 receptor, Nat Chem Biol 16:1105-1110 (2020); Zhang et al., Differential GLP-1R Binding and Activation by Peptide and Non-peptide Agonists, Mol Cell 80:485 (2020).

In embodiments, the GLP-1 receptor agonist is selected from GLP-1, DPP4 degradation resistant GLP-1 (7-37, A8G), exenatide, lixisenatide, albiglutide, dulaglutide, or a variant thereof, having one or more amino acid mutations, independently selected from substitutions, insertions, deletions, and truncations. In embodiments, the GLP-1 receptor agonist has an amino acid sequence of any one of SEQ ID NOs: 57 to 62, 75, and 87-90, or a variant having about 1, 2, 3, 4, 5, 6, 7, 8, 9, or 10 or more amino acid mutations with respect to an amino acid sequence selected from SEQ ID NOs: 57 to 62, 75, and 87-90. In embodiments, the mutations are independently selected from substitutions, insertions, deletions, and truncations. In embodiments, GLP-1 receptor agonist is capable of binding a GLP-1 receptor. In embodiments, the GLP-1 receptor agonist is capable of stimulating insulin secretion and/or inhibiting glucagon secretion.

### FGF19 and FGF21

Fibroblast growth factors (FGFs) are signaling proteins involved in development and metabolism. In humans, there are three endocrine FGFs: FGF19, FGF21, and FGF23.

In embodiments, the chimeric proteins disclosed herein have a general structure of: N terminus—(a)-(b)-(c)—C terminus, wherein (c) is the second domain comprising a fibroblast growth factor 19 (FGF19), FGF21 or a variant thereof, or an analog thereof. In these embodiments, (b) is a linker adjoining the first domain and a second domain, optionally wherein the linker comprises one or more protease-cleavable polypeptide linkers, and/or a hinge-CH2-CH3 Fc domain. In these embodiments, (a) is a first domain comprising a glucagon-like peptide-1 (GLP-1) receptor agonist.

In alternative embodiments, the chimeric proteins disclosed herein have a general structure of: N terminus-(a)-(b)-(c)—C terminus, wherein (a) is the second domain comprising fibroblast FGF19, FGF21 or a variant thereof, or an analog thereof. In these embodiments, (b) is a linker adjoining the first domain and a second domain, optionally wherein the linker comprises one or more protease-cleavable polypeptide linkers, and/or a hinge-CH2-CH3 Fc domain. In these embodiments, (c) is a first domain comprising a glucagon-like peptide-1 (GLP-1) receptor agonist.

In embodiments, the FGF19, FGF21 or a variant thereof, or an analog thereof is the murine FGF15, the ortholog of human FGF19. In embodiments, the FGF19, FGF21 or a variant thereof lack the heparin binding domain common in other FGFs. In embodiments, the FGF19, FGF21 or a variant thereof has low heparan sulphate affinity. Accordingly, in embodiments, the FGF19, FGF21 or a variant thereof enter into the circulatory system and perform endocrine signaling functions.

In embodiments, the FGF19, FGF21 or a variant thereof activate the fibroblast growth factor receptors (FGFRs). In embodiments, the FGF19, FGF21 or a variant thereof require the transmembrane proteins α-Klotho or β-Klotho as a cofactor for signaling. While FGFR proteins are widely expressed, α-Klotho and β-Klotho exhibit tissue specific expression patterns, limiting the tissues where the endocrine FGFs are active. α-Klotho is expressed predominantly in the kidney and brain, and β-Klotho is predominantly expressed in the liver, adipose tissue, and pancreas. Accordingly, in embodiments, the FGF19, FGF21 or a variant thereof binds FGFR-Klotho complexes in an FGF-specific manner (e.g., FGF19 and variants primarily act through FGFR4 and β-Klotho; FGF21 and variants act through FGFR1c and β-Klotho; and FGF23 and variants through FGFR1c with α-Klotho).

FGF19 is produced in the ileum in response to bile acid absorption there. In embodiments, the FGF19 or a variant thereof regulates the production of bile acid. Bile acid is produced and released in the liver, stored in the gall bladder, and released into the duodenum, where it helps emulsify and solubilize fat. In embodiments, the FGF19 or a variant thereof enter circulation, and, in the liver, reduces the expression of the cholesterol 7 alpha-hydroxylase (CYP71A) enzyme, the rate limiting enzyme in bile acid production.

In embodiments, the FGF19, FGF21 or a variant thereof are wild-type human FGF19, FGF21 or FGF23. Suitable FGF19, FGF21 are disclosed in U.S. Pat. Nos. 7,576,190; 8,012,931; 8,541,369; 8,535,912; 8,741,841; 8,883,726; 8,927,492; 8,951,966; 9,089,525; 9,422,353; 9,493,530; 9,889,177; 9,889,178; 9,895,416; 9,974,833; 9,963,494; 9,925,242, and US Publication No. 2007/0237768, the contents of which are hereby incorporated by reference in their entirety.

In embodiments, the FGF19 or a variant thereof is the FGF19 having the following sequence:

(SEQ ID NO: 64)
LAFSDAGPHVHYGWGDPIRLRHLYTSGPHGLSSCFLRIRADGVVDCARG
QSAHSLLEIKAVALRTVAIKGVHSVRYLCMGADGKMQGLLQYSEEDCAF
EEEIRPDGYNVYRSEKHRLPVSLSSAKQRQLYKNRGFLPLSHFLPMLPM
VPEEPEDLRGHLESDMFSSPLETDSMDPFGLVTGLEAVRSPSFEK.

In embodiments, the present chimeric protein comprises FGF19 which has the amino acid sequence of SEQ ID NO: 64. In embodiments, the present chimeric proteins may comprise the FGF19 as described herein, or a variant or functional fragment thereof. For instance, the chimeric protein may comprise a sequence of the FGF19 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the FGF19 as described herein.

In embodiments, the FGF19 reduces bile acid-induced liver damage in choleostatic liver diseases. In embodiments, the FGF19 reduces fat absorption into the body.

In embodiments, the FGF19 or a variant thereof is the adafermin (as M70 or NGM282) having the following sequence:

(SEQ ID NO: 65)
MRDSSPLVHYGWGDPIRLRHLYTSGPHGLSSCFLRIRADGVVDCARGQS
AHSLLEIKAVALRTVAIKGVHSVRYLCMGADGKMQGLLQYSEEDCAFEE
EIRPDGYNVYRSEKHRLPVSLSSAKQRQLYKNRGFLPLSHELPMLPMVP
EEPEDLRGHLESDMFSSPLETDSMDPFGLVTGLEAVRSPSFEK

In embodiments, the present chimeric protein comprises FGF19, FGF21 or a variant thereof is adafermin, which has the amino acid sequence of SEQ ID NO: 65. In embodiments, the present chimeric proteins may comprise adafermin as described herein, or a variant or functional fragment thereof. For instance, the chimeric protein may comprise a sequence of adafermin as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of adafermin as described herein.

In embodiments, the FGF19 or a variant thereof reduces liver fat content (without limitation, e.g., in human NASH patients. In embodiments, the FGF19 or a variant thereof reduces production of bile acid. In embodiments, the fibroblast growth factor comprises FGF19, or an analog thereof. In embodiments, the analog of FGF19 is aldafermin (NGM282). In embodiments, the fibroblast growth factor is capable of activating FGFR4, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence of SEQ ID NOs: 65 or 66.

FGF19 derivatives can be constructed from available structural data, including that described by Harmer et al., The crystal structure of fibroblast growth factor (FGF) 19 reveals novel features of the FGF family and offers a structural basis for its unusual receptor affinity, Biochemistry 43:629-640 (2004); Goetz et al., Molecular insights into the klotho-dependent, endocrine mode of action of fibroblast growth factor 19 subfamily members, Mol Cell Biol 27:3417-3428 (2007); Liu et al., Novel Abs targeting the N-terminus of fibroblast growth factor 19 inhibit hepatocellular carcinoma growth without bile-acid-related side-effects, Cancer Sci 111:1750-1760 (2020); Kuzina et al., Structures of ligand-occupied beta-Klotho complexes reveal a molecular mechanism underlying endocrine FGF specificity and activity, Proc Natl Acad Sci USA 116:7819-7824 (2019).

FGF21 is predominantly expressed in the liver and helps regulate glucose and lipid homeostasis. In embodiments, the chimeric protein reduces weight without decreased caloric intake and improved hepatosteatosis. In embodiments, the chimeric protein reduces glucose levels, body weight, insulin, and cholesterol and triglycerides. Pegbelfermin (BMS-986036) has been shown to reduce liver fat content in human NASH patients.

In embodiments, the FGF21 has the following sequence:

(SEQ ID NO: 66)
HPIPDSSPLLQFGGQVRQRYLYTDDAQQTEAHLEIREDGTVGGAADQSP
ESLLQLKALKPGVIQILGVKTSRFLCQRPDGALYGSLHFDPEACSFREL
LLEDGYNVYQSEAHGLPLHLPGNKSPHRDPAPRGPARFLPLPGLPPALP
EPPGILAPQPPDVGSSDPLSMVGPSQGRSPSYAS

In embodiments, the present chimeric protein comprises FGF21 which has the amino acid sequence of SEQ ID NO: 66. In embodiments, the present chimeric proteins may comprise the FGF21 as described herein, or a variant or functional fragment thereof. For instance, the chimeric protein may comprise a sequence of the FGF21 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the FGF21 as described herein.

In embodiments, the FGF21 or a variant thereof is the FGF21 moiety from efruxifermin (AMG876) having the following sequence:

(SEQ ID NO: 67)
HPIPDSSPLLQFGGQVRQRYLYTDDAQQTEAHLEIREDGTVGGAADQSP
ESLLQLKALKPGVIQILGVKTSRFLCQRPDGALYGSLHFDPEACSFRER
LLEDGYNVYQSEAHGLPLHLPGNKSPHRDPAPRGPARFLPLPGLPPAPP
EPPGILAPQPPDVGSSDPLSMVGGSQGRSPSYES

Efruxifermin has the following amino acid sequence (AMG876; FGF21 moiety underlined, (GGGGS)₃ shown in a boldface font):

(SEQ ID NO: 69)
MDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPGKGGGGSGGGGSGGGGSHP
IPDSSPLLQFGGQVRQRYLYTDDAQQTEAHLEIREDGTVGGAADQSPES
LLQLKALKPGVIQILGVKTSRFLCQRPDGALYGSLHFDPEACSFRERLL
EDGYNVYQSEAHGLPLHLPGNKSPHRDPAPRGPARFLPLPGLPPAPPEP
PGILAPQPPDVGSSDPLSMVGGSQGRSPSYES

In embodiments, the present chimeric protein comprises FGF21 moiety from efruxifermin (AMG876) which has the amino acid sequence of SEQ ID NO: 67. In embodiments, the present chimeric proteins may comprise the FGF21 moiety from efruxifermin (AMG876) as described herein, or a variant or functional fragment thereof. For instance, the chimeric protein may comprise a sequence of the FGF21 moiety from efruxifermin (AMG876) as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the FGF21 moiety from efruxifermin (AMG876) as described herein.

In embodiments, the FGF21 or a variant thereof is LY2405319 having the following amino acid sequence:

(SEQ ID NO: 68)
DSSPLLQFGGQVRQRYLYTDDAQQTEAHLEIREDGTVGGAADQSPESLL
QLKALKPGVIQILGVKTSRFLCQRPDGALYGSLHFDPEACSFRELLLED
GYNVYQSEAHGLPLHCPGNKSPHRDPAPRGPCRFLPLPGLPPALPEPPG
ILAPQPPDVGSSDPLAMVGPSQGRSPSYAS

In embodiments, the present chimeric protein comprises LY2405319, which has the amino acid sequence of SEQ ID NO: 67. In embodiments, the present chimeric proteins may comprise the LY2405319, as described herein, or a variant or functional fragment thereof. For instance, the chimeric protein may comprise a sequence of the LY2405319 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the LY2405319 as described herein.

In embodiments, the FGF21 (RGE), which has the substitutions in comparison with SEQ ID NO: 66 indicated with a boldface font, has the following sequence:

(SEQ ID NO: 76)
HPIPDSSPLLQFGGQVRQRYLYTDDAQQTEAHLEIREDGTVGGAADQSP
ESLLQLKALKPGVIQILGVKTSRFLCQRPDGALYGSLHFDPEACSFRER
LLEDGYNVYQSEAHGLPLHLPGNKSPHRDPAPRGPARFLPLPGLPPALP
EPPGILAPQPPDVGSSDPLSMVGGSQGRSPSYES

In embodiments, the present chimeric protein comprises FGF21 which has the amino acid sequence of SEQ ID NO: 76. In embodiments, the present chimeric proteins may comprise the FGF21 (RGE) as described herein, or a variant or functional fragment thereof. For instance, the chimeric protein may comprise a sequence of the FGF21 (RGE) as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the FGF21 (RGE) as described herein.

In embodiments, the FGF21 (L146P), which has the substitution in comparison with SEQ ID NO: 66 indicated with a boldface font, has the following sequence:

(SEQ ID NO: 77)
HPIPDSSPLLQFGGQVRQRYLYTDDAQQTEAHLEIREDGTVGGAADQSP
ESLLQLKALKPGVIQILGVKTSRFLCQRPDGALYGSLHFDPEACSFREL
LLEDGYNVYQSEAHGLPLHPPGNKSPHRDPAPRGPARFLPLPGLPPALP
EPPGILAPQPPDVGSSDPLSMVGPSQGRSPSYAS

In embodiments, the present chimeric protein comprises FGF21 which has the amino acid sequence of SEQ ID NO: 77. In embodiments, the present chimeric proteins may comprise the FGF21 as described herein, or a variant or functional fragment thereof. For instance, the chimeric protein may comprise a sequence of the FGF21 (L146P) as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the FGF21 (L146P) as described herein.

In embodiments, the fibroblast growth factor comprises FGF21, or an analog thereof. In embodiments, the analog of FGF21 is selected from efruxifermin, LY2405319, FGF21 (RGE) and FGF21 (L146P). In embodiments, the fibroblast growth factor is capable of activating FGFR1c, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence selected from SEQ ID NOs: 66 to 68, 76 and 77.

In embodiments, the analog of FGF21 is the FGF21 moiety from pegbelfermin (BMS-986036) or an amino acid sequence that is at least about 90%, or at least about 95%, or at least about 97%, or at least about 98%, or at least about 99% identity thereto.

FGF21 derivatives can be constructed from available structural data, including that described by Lee et al., Structures of beta-klotho reveal a ‘zip code’-like mechanism for endocrine FGF signaling, Nature 553:501-505 (2018); Kharitonenkov et al., Rational Design of a Fibroblast Growth Factor 21-Based Clinical Candidate, LY2405319. PLOS ONE 8(3):e58575 (2013); and Huang, J. et al., Development of a Novel Long-Acting Antidiabetic FGF21 Mimetic by Targeted Conjugation to a Scaffold Antibody, The Journal Of Pharmacology And Experimental Therapeutics 346(2):270-280 (2013).

### Linker

In embodiments, the chimeric protein comprises a linker.

In embodiments, the chimeric proteins disclosed herein have a general structure of: N terminus—(a)-(b)-(c)—C terminus, wherein (a) is a first domain comprising a glucagon-like peptide-1 (GLP-1) receptor agonist and (b) is a linker adjoining the first domain and a second domain, optionally wherein the linker comprises one or more protease-cleavable polypeptide linkers, and/or a hinge-CH2-CH3 Fc domain. In these embodiments, (c) is the second domain comprising a fibroblast growth factor 19 (FGF19), FGF21 or a variant thereof, or an analog thereof.

In alternative embodiments, the chimeric proteins disclosed herein have a general structure of: N terminus-(a)-(b)-(c)—C terminus, wherein (c) is a first domain comprising a glucagon-like peptide-1 (GLP-1) receptor agonist, and (b) is a linker adjoining the first domain and a second domain, optionally wherein the linker comprises one or more protease-cleavable polypeptide linkers, and/or a hinge-CH2-CH3 Fc domain. In these embodiments, (a) is the second domain comprising a fibroblast growth factor 19 (FGF19), FGF21 or a variant thereof, or an analog thereof.

In embodiments, the present chimeric proteins may comprise variants of the protease-cleavable polypeptide linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50 or 79.

TABLE 1
Illustrative protease-cleavable polypeptide
linkers

| SEQ ID NO. | Sequence |
|---|---|
| 70 | HSSKLQ |
| 71 | GPLGVRG |
| 72 | IPVSLRSG |
| 73 | VPLSLYSG |
| 74 | SGESPAYYTA |
| 78 | RFRS |

In embodiments, the protease-cleavable polypeptide linkers are cleavable by a protease that is endogenous to a mammalian liver. In embodiments, the protease is selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. Accordingly, in embodiments, the protease that cleaves the protease-cleavable polypeptide linkers is already present in the subject and an exogenous protease need not be administered. In embodiments, levels of the protease are elevated by liver injury, nonalcoholic fatty liver disease (NAFLD), non-alcoholic steatohepatitis (NASH), and/or fibrosis. In embodiments, the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74. In embodiments, the protease-cleavable polypeptide linker is C terminal to the first domain or N terminal to the second domain. Additional suitable protease-cleavable polypeptide linkers are disclosed in US Publication Nos. 2009/0042787 and 2021/0130430, the contents of which are hereby incorporated by reference in their entirety.

In embodiments, the first domain comprising a glucagon-like peptide-1 (GLP-1) receptor agonist and the protease-cleavable polypeptide linker is C terminal to the first domain; or the second domain comprises a glucagon-like peptide-1 (GLP-1) receptor agonist and the protease-cleavable polypeptide linker is N terminal to the second domain. In embodiments, the chimeric protein comprises two protease-cleavable polypeptide linkers, such protease-cleavable polypeptide linker independently selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74. In embodiments, the first protease-cleavable polypeptide linker is C terminal to the first domain and the second protease-cleavable polypeptide linker is N terminal to the second domain.

In embodiments, the linker comprises a protease-cleavable polypeptide linker. In embodiments, the protease-cleavable polypeptide linker cleavable by a protease that is endogenous to a mammalian liver. In embodiments, the protease-cleavable linker is cleavable by a protease selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the protease-cleavable linker comprises a consensus recognition and/or cleavage site of a protease selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

In embodiments, the chimeric protein comprises two protease-cleavable polypeptide linkers. In embodiments, wherein the first protease-cleavable polypeptide linker is C terminal to the first domain and the second protease-cleavable polypeptide linker is N terminal to the second domain. In embodiments, the two protease-cleavable polypeptide linkers are cleavable by a protease that is endogenous to a mammalian liver. In embodiments, the two protease-cleavable polypeptide linkers are cleavable by a protease independently selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers comprise consensus recognition and/or cleavage sites of a proteases independently selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers are cleavable by a protease independently comprises an amino acid sequence selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

In embodiments, the linker comprises at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present disclosure, the linker is a polypeptide selected from a flexible amino acid sequence, an IgG hinge region, or an antibody sequence. In embodiments, the linker comprises hinge-CH2-CH3 Fc domain derived from IgG4, optionally human IgG4. In embodiments, the linker comprises hinge-CH2-CH3 Fc domain derived from IgG1, optionally human IgG1.

In embodiments, the linker may be derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker is a synthetic linker such as PEG.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1> IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2).

In a chimeric protein of the present disclosure, the linker comprises a hinge-CH2-CH3 Fc domain derived from IgG4. In embodiments, the linker comprises a hinge-CH2-CH3 Fc domain derived from a human IgG4. In embodiments, the linker has at least about 95%, or at least about 97%, or at least about 97%, or at least about 98% sequence identity with the amino acid sequence of any one of SEQ ID NO: 1 to SEQ ID NO: 3 or SEQ ID NO: 79, e.g., at least 95% identical to the amino acid sequence of SEQ ID NO: 2. In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, I253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an I253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

In embodiments, the Fc domain in a linker comprises the amino acid sequence of SEQ ID NO: 1 (see the below table), or at least about 90%, or at least about 93%, or at least about 95%, or at least about 97%, or at least about 98%, or at least about 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see the below table), or at least about 90%, or at least about 93%, or at least about 95%, or at least about 97%, or at least about 98%, or at least about 99% identity thereto. An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e., other than FcRn) with effector function.

In embodiments, the Fc domain in a linker has the amino acid sequence of SEQ ID NO: 1 (see Table 2, below), or at least at least about 90%, or at least about 93%, or at least about 95%, or at least about 97%, or at least about 98%, or at least about 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 2, below), or at least at least about 90%, or at least about 93%, or at least about 95%, or at least about 97%, or at least about 98%, or at least about 99% identity thereto. For instance, in embodiments, the Fc domain in a linker comprises the amino acid sequence of SEQ ID NO: 3 (see Table 2, below), or at least at least about 90%, or at least about 93%, or at least about 95%, or at least about 97%, or at least about 98%, or at least about 99% identity thereto.

In embodiments, the linker comprises a hinge-CH2-CH3 Fc domain derived from IgG1. In embodiments, the IgG1 is human IgG1. In embodiments, the linker comprises a hinge-CH2-CH3 Fc domain derived from IgG4. In embodiments, the IgG4 is human IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 79. In embodiments, the linker further comprises the linker comprises one or more joining linkers, such joining linkers independently selected from SEQ ID NOs: 4 to 50 or 79. In embodiments, the linker comprises two or more joining linkers each joining linker independently selected from SEQ ID NOs: 4 to 50 or 79; wherein one joining linker is N terminal to the hinge-CH2-CH3-Fc domain and another joining linker is C terminal to the hinge-CH2-CH3-Fc domain.

Further, one or more joining linkers may be employed to connect an Fc domain in a linker (e.g., one of SEQ ID NO: 1, SEQ ID NO: 2, SEQ ID NO: 3 or at least at least about 90%, or at least about 93%, or at least about 95%, or at least about 97%, or at least about 98%, or at least about 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50 or 79, or variants thereof are located between an extracellular domain as disclosed herein and an Fc domain as disclosed herein.

In embodiments, the present chimeric proteins may comprise variants of the joining linkers disclosed in Table 2, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50 or 79.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatamers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NOs: 4 to 50 or 79 and are provided in Table 2 below:

TABLE 2
Illustrative linkers (Fc domain linkers and joining linkers)

| SEQ ID | |
|---|---|
| NO. | Sequence |
| 1 | APEFLGGPSVFLFPPKPKDTLMISRTPEVTCVVVDVSQEDPEVQFNWYVDGVEVHNAKTK |
| | PREEQFNSTYRVVSVLTVLHQDWLSGKEYKCKVSSKGLPSSIEKTISNATGQPREPQVYTL |
| | PPSQEEMTKNQVSLTCLVKGFYPSDIAVEWESNGQPENNYKTTPPVLDSDGSFFLYSRLTV |
| | DKSSWQEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTK |
| | PREEQFNSTYRVVSVLTTPHSDWLSGKEYKCKVSSKGLPSSIEKTISNATGQPREPQVYTL |
| | PPSQEEMTKNQVSLTCLVKGFYPSDIAVEWESNGQPENNYKTTPPVLDSDGSFFLYSRLTV |
| | DKSSWQEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTK |
| | PREEQFNSTYRVVSVLTVLHQDWLSGKEYKCKVSSKGLPSSIEKTISNATGQPREPQVYTL |
| | PPSQEEMTKNQVSLTCLVKGFYPSDIAVEWESNGQPENNYKTTPPVLDSDGSFFLYSRLTV |
| | DKSRWQEGNVFSCSVLHEALHNHYTQKSLSLSLGK |
| 79 | PCPAPEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GGS |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |
| 80 | EPKSCDKTHTCP |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS (SEQ ID NO: 19. In embodiments, the joining linker is GS or LE. In embodiments, the joining linker is EPKSCDKTHTCP (SEQ ID NO: 80).

In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, where a chimeric protein comprises a glucagon-like peptide-1 (GLP-1), a protease-cleavable linker preceding the Fc domain, an Fc domain, a joining linker following the Fc domain, and FGF19, the chimeric protein may comprise the following structure:
Glucagon-like peptide-1 (GLP-1)—a protease-cleavable linker—Fc Domain—Joining Linker—FGF19

In embodiments, where a chimeric protein comprises FGF19, a joining linker preceding the Fc domain, an Fc domain, a protease-cleavable linker following the Fc domain, a glucagon-like peptide-1 (GLP-1), and the chimeric protein may comprise the following structure:
FGF19—Joining Linker—Fc Domain—a protease-cleavable linker—glucagon-like peptide-1 (GLP-1)

In embodiments, where a chimeric protein comprises a glucagon-like peptide-1 (GLP-1), a protease-cleavable linker preceding the Fc domain, an Fc domain, a joining linker following the Fc domain, and FGF21, the chimeric protein may comprise the following structure:
Glucagon-like peptide-1 (GLP-1)—a protease-cleavable linker—Fc Domain—Joining Linker—FGF21

In embodiments, where a chimeric protein comprises FGF21, a joining linker preceding the Fc domain, an Fc domain, a protease-cleavable linker following the Fc domain, a glucagon-like peptide-1 (GLP-1), and the chimeric protein may comprise the following structure:
FGF21—Joining Linker—Fc Domain—a protease-cleavable linker—glucagon-like peptide-1 (GLP-1)

In embodiments, a chimeric protein comprises only one joining linkers. In embodiments, a chimeric protein comprises only two joining linkers. In embodiments, a chimeric protein lacks joining linkers.

An illustrative GLP-1-Fc-FGF19 chimeric protein has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF19 is shown in an italics font, with the mutations therein shown in a boldface-italics font):

(SEQ ID NO: 81)
HGEGTFTSDVSSYLEEQAAKEFIAWLVKGRGEPKSCDKTHTCPPCPAPE
AAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMH
EALHNHYTQKSLSLSPGKIEGRMDMRDSSPLVHYGWGDPIRLRHLYTSG
PHGLSSCFLRIRADGVVDCARGQSAHSLLEIKAVALRTVAIKGVHSVRY
LCMGADGKMQGLLQYSEEDCAFEEEIRPDGYNVYRSEKHRLPVSLSSAK
QRQLYKNRGFLPLSHFLPMLPMVPEEPEDLRGHLESDMFSSPLETDSMD
PFGLVTGLEAVRSPSFEK

In embodiments, the chimeric protein comprises a variant of the GLP-1-Fc-FGF19 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

An illustrative GLP-1-Fc-FGF21 (RGE) chimeric protein has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF21 (RGE) is shown in an italics font, with the mutations therein shown in a boldface-italics font):

(SEQ ID NO: 82)
HGEGTFTSDVSSYLEEQAAKEFIAWLVKGRGEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKIEGRMDHPIPDSSPLL
QFGGQVRQRYLYTDDAQQTEAHLEIREDGTVGGAADQSPESLLQLKALKPGVIQILGVKTSRFLCQRPDGA
LYGSLHFDPEACSFRERLLEDGYNVYQSEAHGLPLHLPGNKSPHRDPAPRGPARFLPLPGLPPALPEPPGIL
APQPPDVGSSDPLSMVGGSQGRSPSYES

In embodiments, the chimeric protein comprises a variant of the GLP-1-Fc-FGF21 (RGE) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

An illustrative GLP-1-Fc-FGF21 (L146P) chimeric protein has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF21 (L146P) is shown in an italics font, with the mutations therein shown in a boldface-italics font):

(SEQ ID NO: 83)
HGEGTFTSDVSSYLEEQAAKEFIAWLVKGRGEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKIEGRMDHPIPDSSPLL
QFGGQVRQRYLYTDDAQQTEAHLEIREDGTVGGAADQSPESLLQLKALKPGVIQILGVKTSRFLCQRPDGA
LYGSLHFDPEACSFRELLLEDGYNVYQSEAHGLPLHPPGNKSPHRDPAPRGPARFLPLPGLPPALPEPPGIL
APQPPDVGSSDPLSMVGPSQGRSPSYAS

In embodiments, the chimeric protein comprises a variant of the GLP-1-Fc-FGF21 (L146P) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

An illustrative GLP-1 (RSRF)-Fc-FGF19 chimeric protein has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF19 is shown in an italics font, with the mutations therein shown in a boldface-italics font):

(SEQ ID NO: 84)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDMRDSS
PLVHYGWGDPIRLRHLYTSGPHGLSSCFLRIRADGVVDCARGQSAHSLLEIKAVALRTVAIKGVHSVRYLCM
GADGKMQGLLQYSEEDCAFEEEIRPDGYNVYRSEKHRLPVSLSSAKQRQLYKNRGFLPLSHELPMLPMVP
EEPEDLRGHLESDMFSSPLETDSMDPFGLVTGLEAVRSPSFEK

In embodiments, the chimeric protein comprises a variant of the GLP-1 (RSRF)-Fc-FGF19 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84.

An illustrative GLP-1 (RSRF)-Fc-FGF21 (RGE) chimeric protein has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF21 (RGE) is shown in an italics font, with the mutations therein shown in a boldface-italics font):

(SEQ ID NO: 85)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDHPIPDS
SPLLQFGGQVRQRYLYTDDAQQTEAHLEIREDGTVGGAADQSPESLLQLKALKPGVIQILGVKTSRFLCQR
PDGALYGSLHFDPEACSFRERLLEDGYNVYQSEAHGLPLHLPGNKSPHRDPAPRGPARFLPLPGLPPALPE
PPGILAPQPPDVGSSDPLSMVGGSQGRSPSYES

In embodiments, the chimeric protein comprises a variant of the GLP-1 (RSRF)-Fc-FGF21 (RGE) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85.

An illustrative GLP-1 (RSRF)-Fc-FGF21 (L146P) chimeric protein has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF21 (L146P) is shown in an italics font, with the mutations therein shown in a boldface-italics font):

(SEQ ID NO: 86)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDHPIPDS
SPLLQFGGQVRQRYLYTDDAQQTEAHLEIREDGTVGGAADQSPESLLQLKALKPGVIQILGVKTSRFLCQR
PDGALYGSLHFDPEACSFRELLLEDGYNVYQSEAHGLPLHPPGNKSPHRDPAPRGPARFLPLPGLPPALPE
PPGILAPQPPDVGSSDPLSMVGPSQGRSPSYAS

In embodiments, the chimeric protein comprises a variant of the GLP-1 (RSRF)-Fc-FGF21 (L146P) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86.

### Isolated Polynucleotide Encoding the Chimeric Protein

In aspects, the present disclosure provides an isolated polynucleotide encoding the chimeric protein of any one of the embodiments disclosed herein.

Accordingly, In aspects, the present disclosure provides an isolated polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist. In embodiments, the chimeric protein is administered to a patient. In embodiments, a nucleic acid encoding the chimeric protein (e.g., modified mRNA or DNA) is administered to a patient. In embodiments, the chimeric protein the glucagon-like peptide-1 (GLP-1) receptor agonist of any of the embodiments disclosed herein. In embodiments, the chimeric protein the fibroblast growth factor 19 (FGF19), FGF21, FGF23, or a variant thereof, or an analog thereof of any of the embodiments disclosed herein. In embodiments, the nucleic acid encoding harbors control elements that enable the expression of the chimeric protein (e.g., modified mRNA or DNA) is the liver.

Each of glucagon-like peptide-1 (GLP-1), fibroblast growth factor 19 (FGF19), fibroblast growth factor 21 (FGF21) have a very short half-life, limiting their potential direct use as therapeutics. For example, GLP-1 secreted in the blood has a very short half-life of less than 2 minutes, which is caused by a loss of activity due to the cleavage of amino acids at the N-terminus by the enzyme dipeptidyl peptidase-4 (DPP-4). Similarly, FGF19 and FGF21 has a very short half-life of ˜0.5-2 hours. Therefore, novel approaches to deliver GLP-1, FGF19 and/or FGF21 are required. The present disclosure addresses this need by delivering pharmaceutical compositions which can contain nucleic acids such as modified mRNA (mmRNA) or DNA.

In aspects, the present disclosure provides an isolated polynucleotide encoding a chimeric protein having a general structure of: N terminus—(a)-(b)-(c)—C terminus, wherein: (a) is a first domain comprising a glucagon-like peptide-1 (GLP-1); (b) is a linker adjoining the first domain and a second domain comprising a hinge-CH2-CH3 Fc domain, and (c) is FGF19. In embodiments, the isolated polynucleotide encodes a chimeric protein comprises a glucagon-like peptide-1 (GLP-1), a protease-cleavable linker preceding the Fc domain, an Fc domain, a joining linker following the Fc domain, and FGF19, wherein the chimeric protein may comprise the following structure:
Glucagon-like peptide-1 (GLP-1)—a protease-cleavable linker—Fc Domain—Joining Linker—FGF19

In aspects, the present disclosure provides an isolated polynucleotide encoding a chimeric protein having a general structure of: N terminus—(a)-(b)-(c)—C terminus, wherein: (a) is FGF19; (b) is a linker adjoining the first domain and a second domain comprising a hinge-CH2-CH3 Fc domain, and (c) is GLP-1. In embodiments, the isolated polynucleotide encodes a chimeric protein comprises FGF19, a joining linker preceding the Fc domain, an Fc domain, a protease-cleavable linker following the Fc domain, a glucagon-like peptide-1 (GLP-1), and the chimeric protein may comprise the following structure:
FGF19—Joining Linker—Fc Domain—a protease-cleavable linker—glucagon-like peptide-1 (GLP-1)

In aspects, the present disclosure provides an isolated polynucleotide encoding a chimeric protein having a general structure of: N terminus—(a)-(b)-(c)—C terminus, wherein: (a) is a first domain comprising a glucagon-like peptide-1 (GLP-1); (b) is a linker adjoining the first domain and a second domain comprising a hinge-CH2-CH3 Fc domain, and (c) is FGF21. In embodiments, the isolated polynucleotide encodes a chimeric protein comprises a glucagon-like peptide-1 (GLP-1), a protease-cleavable linker preceding the Fc domain, an Fc domain, a joining linker following the Fc domain, and FGF21, the chimeric protein may comprise the following structure:
Glucagon-like peptide-1 (GLP-1)—a protease-cleavable linker—Fc Domain—Joining Linker—FGF21

In aspects, the present disclosure provides an isolated polynucleotide encoding a chimeric protein having a general structure of: N terminus—(a)-(b)-(c)—C terminus, wherein: (a) is a first domain comprising FGF21; (b) is a linker adjoining the first domain and a second domain comprising a hinge-CH2-CH3 Fc domain, and (c) is a glucagon-like peptide-1 (GLP-1). In embodiments, the isolated polynucleotide encodes a In embodiments, where a chimeric protein comprises FGF21, a joining linker preceding the Fc domain, an Fc domain, a protease-cleavable linker following the Fc domain, a glucagon-like peptide-1 (GLP-1), and the chimeric protein may comprise the following structure:
FGF21—Joining Linker—Fc Domain—a protease-cleavable linker—glucagon-like peptide-1 (GLP-1)

In embodiments, a chimeric protein comprises only one joining linkers. In embodiments, a chimeric protein comprises only two joining linkers. In embodiments, a chimeric protein lacks joining linkers.

An illustrative GLP-1-Fc-FGF19 chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF19 is shown in an italics font, with the mutations therein shown in a boldface-italics font):

(SEQ ID NO: 81)
HGEGTFTSDVSSYLEEQAAKEFIAWLVKGRGEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKIEGRMDMRDSSPLVH
YGWGDPIRLRHLYTSGPHGLSSCFLRIRADGVVDCARGQSAHSLLEIKAVALRTVAIKGVHSVRYLCMGADG
KMQGLLQYSEEDCAFEEEIRPDGYNVYRSEKHRLPVSLSSAKQRQLYKNRGFLPLSHELPMLPMVPEEPE
DLRGHLESDMFSSPLETDSMDPFGLVTGLEAVRSPSFEK

In embodiments, the isolated polynucleotide of the present disclosure encode a chimeric protein that comprises a variant of the GLP-1-Fc-FGF19 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

An illustrative GLP-1-Fc-FGF21 (RGE) chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF21 (RGE) is shown in an italics font, with the mutations therein shown in a boldface-italics font):

(SEQ ID NO: 82)
HGEGTFTSDVSSYLEEQAAKEFIAWLVKGRGEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKIEGRMDHPIPDSSPLL
QFGGQVRQRYLYTDDAQQTEAHLEIREDGTVGGAADQSPESLLQLKALKPGVIQILGVKTSRFLCQRPDGA
LYGSLHFDPEACSFRERLLEDGYNVYQSEAHGLPLHLPGNKSPHRDPAPRGPARFLPLPGLPPALPEPPGIL
APQPPDVGSSDPLSMVGGSQGRSPSYES

In embodiments, the isolated polynucleotide of the present disclosure encodes a variant of the GLP-1-Fc-FGF21 (RGE) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

An illustrative GLP-1-Fc-FGF21 (L146P) chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a joining linker is shown in an underlined-boldface-italic font, and FGF21 (L146P) is shown in an italics font, with the mutations therein shown in a boldface-italics font):

(SEQ ID NO: 83)
HGEGTFTSDVSSYLEEQAAKEFIAWLVKGRGEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKIEGRMDHPIPDSSPLL
QFGGQVRQRYLYTDDAQQTEAHLEIREDGTVGGAADQSPESLLQLKALKPGVIQILGVKTSRFLCQRPDGA
LYGSLHFDPEACSFRELLLEDGYNVYQSEAHGLPLHPPGNKSPHRDPAPRGPARFLPLPGLPPALPEPPGIL
APQPPDVGSSDPLSMVGPSQGRSPSYAS

In embodiments, the isolated polynucleotide of the present disclosure encodes a variant of the GLP-1-Fc-FGF21 (L146P) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

An illustrative GLP-1 (RSRF)-Fc-FGF19 chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF19 is shown in an italics font, with the mutations therein shown in a boldface-italics font):

(SEQ ID NO: 84)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDMRDSS
PLVHYGWGDPIRLRHLYTSGPHGLSSCFLRIRADGVVDCARGQSAHSLLEIKAVALRTVAIKGVHSVRYLCM
GADGKMQGLLQYSEEDCAFEEEIRPDGYNVYRSEKHRLPVSLSSAKQRQLYKNRGFLPLSHELPMLPMVP
EEPEDLRGHLESDMFSSPLETDSMDPFGLVTGLEAVRSPSFEK

In embodiments, the isolated polynucleotide of the present disclosure encodes a variant of the GLP-1 (RSRF)-Fc-FGF19 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84.

An illustrative GLP-1 (RSRF)-Fc-FGF21 (RGE) chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF21 (RGE) is shown in an italics font, with the mutations therein shown in a boldface-italics font):

(SEQ ID NO: 85)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDHPIPDS
SPLLQFGGQVRQRYLYTDDAQQTEAHLEIREDGTVGGAADQSPESLLQLKALKPGVIQILGVKTSRFLCQR
PDGALYGSLHFDPEACSFRERLLEDGYNVYQSEAHGLPLHLPGNKSPHRDPAPRGPARFLPLPGLPPALPE
PPGILAPQPPDVGSSDPLSMVGGSQGRSPSYES

In embodiments, the isolated polynucleotide of the present disclosure encodes a variant of the GLP-1 (RSRF)-Fc-FGF21 (RGE) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85.

An illustrative GLP-1 (RSRF)-Fc-FGF21 (L146P) chimeric protein that an isolated polynucleotide of the present disclosure encode has the following sequence (GLP-1 is shown by an underline, a linker comprising a mutant Fc domain of human IgG1 is shown in boldface font, joining linkers are shown in a boldface-underlined font, a protease-cleavable linker is shown in an unmarked font, a joining linker is shown in an underlined-boldface-italic font, and FGF21 (L146P) is shown in an italics font, with the mutations therein shown in a boldface-italics font):

(SEQ ID NO: 86)
HGEGTFTSDVSSYLEEQAAKEFIAWLVKGRGRFRSEPKSCDKTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPGKIEGRMDHPIPDS
SPLLQFGGQVRQRYLYTDDAQQTEAHLEIREDGTVGGAADQSPESLLQLKALKPGVIQILGVKTSRFLCQR
PDGALYGSLHFDPEACSFRELLLEDGYNVYQSEAHGLPLHPPGNKSPHRDPAPRGPARFLPLPGLPPALPE
PPGILAPQPPDVGSSDPLSMVGPSQGRSPSYAS

In embodiments, the isolated polynucleotide of the present disclosure encode a variant of the GLP-1 (RSRF)-Fc-FGF21 (L146P) chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86.

In embodiments, the polynucleotide is RNA, optionally, an mRNA. In embodiments, the polynucleotide is codon optimized.

In embodiments, the polynucleotide is mRNA or a modified mRNA (mmRNA). In embodiments, the polynucleotide may include a polynucleotide modification including, but not limited to, a nucleoside modification. In embodiments, the polynucleotide is an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

In embodiments, the polypeptide the at least one chemically modified nucleoside is selected from pseudouridine (4), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), .alpha.-thio-guanosine, .alpha.-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m11), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof.

In embodiments, the mmRNA does not cause a substantial induction of the innate immune response of a cell into which the mmRNA is introduced. In embodiments, the modification in the mmRNA enhance one or more of the efficiency of production of the chimeric protein, intracellular retention of the mmRNA, and viability of contacted cells, and possess reduced immunogenicity.

In embodiments, the mmRNA has a length sufficient to include an open reading frame encoding the chimeric protein of the present disclosure.

In embodiments, the mmRNA is not uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

In embodiments, the mmRNA may contain a modified pyrimidine such as uracil or cytosine. In embodiments, at least about 5%, at least about 10%, at least about 25%, at least about 50%, In embodiments, the modified uracil may be replaced by a compound having a single unique structure, or can be replaced by a plurality of compounds having different structures disclosed above (e.g., same mmRNA may contain 2, 3, 4 or more types of uniquely modified uracil). In embodiments, at least about 5%, at least about 10%, at least about 25%, at least about 50%, at least about 80%, at least about 90% or 100% of the cytosine in the nucleic acid may be replaced with a modified cytosine. The modified cytosine can be replaced by a compound having a single unique structure, or can be replaced by a plurality of compounds having different structures disclosed above (e.g., same mmRNA may contain 2, 3, 4 or more types of uniquely modified cytosine).

In embodiments, the mmRNA comprises at least one chemically modified nucleoside. In embodiments, wherein the at least one chemically modified nucleoside is selected from pseudouridine (Y), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), .alpha.-thio-guanosine, .alpha.-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m11), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In embodiments, the mmRNA comprises at least one chemically modified nucleoside, wherein the at least one chemically modified nucleoside is selected from pseudouridine, N1-methylpseudouridine, 5-methylcytosine, 5-methoxyuridine, and a combination thereof. In embodiments, the mmRNA comprises at least one chemically modified nucleoside is N1-methylpseudouridine. In embodiments, the mmRNA is fully modified with chemically-modified uridines. In embodiments, the mmRNA is a fully modified N1-methylpseudouridine mRNA. Additional chemical modifications are disclosed in US Patent Application Publication No. 20190111003, the entire contents of which are hereby incorporated by reference.

In embodiments, modified nucleosides include pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, modified nucleosides include 2-aminopurine, 2,6-diaminopurine, 6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In embodiments, the nucleotide can be modified on the major groove face and can include replacing hydrogen on C-5 of uracil with a methyl group or a halo group.

In embodiments, a modified nucleoside is 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine or 5′-O-(1-Thiophosphate)-Pseudouridine.

Further examples of modified nucleotides and modified nucleotide combinations are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, and US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO/2017112943, WO 2014/028429, WO 2017/201325 the entire contents of which are hereby incorporated by reference. The methods for synthesizing the modified mRNA are disclosed, e.g., in US Patent Application Publication Nos. 20170204152, the entire contents of which are hereby incorporated by reference.

In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues of the mmRNA are replaced by a modified cytosine residues. In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the uracil residues of the mmRNA are replaced by modified uracil residues. In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the adenine residues of the mmRNA are replaced by modified adenine residues. In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the guanine residues of the mmRNA are replaced by modified guanine residues.

In embodiments, the mmRNA further comprises a 5′ untranslated region (UTR) and/or a 3′UTR, wherein either or both may independently contain one or more different nucleoside modifications. In such embodiments, nucleoside modifications may also be present in the translatable region. In embodiments, the mmRNA further comprises a Kozak sequence. In embodiments, the mmRNA further comprises a internal ribosome entry site (IRES).

In embodiments, the mmRNA further comprises a 5′-cap and/or a poly A tail.

In embodiments, the 5′-cap contains a 5′-5′-triphosphate linkage between the 5′-most nucleotide and guanine nucleotide. In embodiments, the 5′-cap comprises a methylation of the ultimate and penultimate most 5′-nucleotides on the 2′-hydroxyl group. In embodiments, the 5′-cap facilitates binding the mRNA Cap Binding Protein (CBP), confers mRNA stability in the cell and/or confers translation competency.

In embodiments, the poly-A tail is greater than about 30 nucleotides, or greater than about 40 nucleotides in length. In embodiments, the poly-A tail at least about 40 nucleotides, or at least about 45 nucleotides, or at least about 55 nucleotides, or at least about 60 nucleotides, or at least about 80 nucleotides, or at least about 90 nucleotides, or at least about 100 nucleotides, or at least about 120 nucleotides, or at least about 140 nucleotides, or at least about 160 nucleotides, or at least about 180 nucleotides, or at least about 200 nucleotides, or at least about 250 nucleotides, or at least about 300 nucleotides, or at least about 350 nucleotides, or at least about 400 nucleotides, or at least about 450 nucleotides, or at least about 500 nucleotides, or at least about 600 nucleotides, or at least about 700 nucleotides, or at least about 800 nucleotides, or at least about 900 nucleotides, or at least about 1000 nucleotides in length.

In embodiments, the mmRNA comprises a 3′ untranslated region (UTR). In embodiments, the 3′ UTR comprises a nucleic acid sequence at least about 90%, or at least about 95%, or at least about 96%, or at least about 97%, or at least about 98%, or at least about 99%, or about 100% identical to a sequence listed in Table 4A or Table 4B of US Patent Application Publication No. 20190114089, which is incorporated herein in its entirety. In embodiments, the 3′ UTR comprises at least one microRNA-122 (miR-122) binding site, wherein the miR-122 binding site is a miR-122-3p binding site or a miR-122-5-binding site. In embodiments, the mmRNA comprises a nucleic acid sequence comprising a miRNA binding site. In some embodiments, the miRNA binding site binds to miR-122. In a particular embodiment, the miRNA binding site binds to miR-122-3p or miR-122-5p. In embodiments, the mmRNA comprises at least two, at least three, at least four, at least five, at least six, at least seven, at least eight, at least nine, or at least ten miRNA binding sites.

In embodiments, the 3′ UTR sequence is derived from human hemoglobin subunit beta, which has the following nucleotide sequence:

(SEQ ID NO: 91)
GCTCGCTTTCTTGCTGTCCAATTTCTATTAAAGGTTCCTTTGTTCCCTAAGTCCAACTACTAAACTGGGG
GATATTATGAAGGGCCTTGAGCATCTGGATTCTGCCTAATAAAAAACATTTATTTTCATTGCAATTGCCATG
TGTATGTGGGTTCGCCCACATACTCTGATGATCCCCAATCGTGGCGTGTCGGCCTGCTTCGGCAGGCA
CTGGCGCCGGGATCATTCATGGCAA

In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 91. In embodiments, the 3′ UTR comprises a nucleotide acid sequence that is at least about 90%, or at least about 95%, or at least about 96%, or at least about 97%, or at least about 98%, or at least about 99%, or 100% identical to the nucleotide acid sequence of SEQ ID NO: 91 with one or more T nucleotide replaced with a U nucleotide, a modified U nucleotide or a homolog thereof. In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 91 with at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% T nucleotide replaced with a U nucleotide, a modified U nucleotide or a homolog thereof. In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 91 at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues, thymidine residues, adenosine residues and/or guanosine residues are replaced by modified cytidine residues, modified uridine residues, modified adenosine residues and/or modified guanosine residues, respectively.

In embodiments, the 3′ UTR sequence is derived from a dengue virus UTR, which has the following nucleotide sequence:

(SEQ ID NO: 92)
AAAGCAAAACTAACATGAAACAAGGCTAGAAGTCAGGTCGGATTAAGCCATAGTACGGAAAAAACTATG
CTACCTGTGAGCCCCGTCCAAGGACGTTAAAAGAAGTCAGGCCATCATAAATGCCATAGCTTGAGTAAA
CTATGCAGCCTGTAGCTCCACCTGAGAAGGTGTAAAAAATCCGGGAGGCCACAAACCATGGAAGCTGT
ACGCATGGCGTAGTGGACTAGCGGTTAGAGGAGACCCCTCCCTTACAAATCGCAGCAACAATGGGGGC
CCAAGGCGAGATGAAGCTGTAGTCTCGCTGGAAGGACTAGAGGTTAGAGGAGACCCCCCCGAAACAA
AAAACAGCATATTGACGCTGGGAAAGACCAGAGATCCTGCTGTCTCCTCAGCATCATTCCAGGCACAG
AACGCCAGAAAATGGAATGGTGCTGTTGAATCAACAGGTTCT

In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 92. In embodiments, the 3′ UTR comprises a nucleotide acid sequence that is at least about 90%, or at least about 95%, or at least about 96%, or at least about 97%, or at least about 98%, or at least about 99%, or 100% identical to the nucleotide acid sequence of SEQ ID NO: 92 with one or more T nucleotide replaced with a U nucleotide, a modified U nucleotide or a homolog thereof. In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 92 with at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% T nucleotide replaced with a U nucleotide, a modified U nucleotide or a homolog thereof. In embodiments, the 3′ UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 92 at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues, thymidine residues, adenosine residues and/or guanosine residues are replaced by modified cytidine residues, modified uridine residues, modified adenosine residues and/or modified guanosine residues, respectively.

In embodiments, the miRNA binding site is inserted within the 3′ UTR. In embodiments, the polynucleotide is DNA. In embodiments, the further comprises a spacer sequence between the open reading frame and the miRNA binding site. In aspects, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR comprises a nucleic acid sequence at least about 90%, or at least about 95%, or at least about 96%, or at least about 97%, or at least about 98%, or at least about 99%, or about 100% identical to a sequence listed in Table 3 of US Patent Application Publication No. 20190114089, or a sequence disclosed in PCT International Application Publication Nos. WO 2017/201325 and WO 2014/164253, each of which is incorporated herein in its entirety. In embodiments, the 5′ UTR bears features, which play roles in translation initiation. In embodiments, the 5′ UTR harbors signatures like Kozak sequences which are commonly known to be involved in the process by which the ribosome initiates translation of many genes. In embodiments, the 5′ UTR forms secondary structures which are involved in elongation factor binding. In embodiments, the 5′ UTR of mRNA known to be upregulated in cancers, such as c-myc, may be used to enhance expression of a nucleic acid molecule, such as a polynucleotides, in cancer cells. In embodiments, the 5′ UTR of mRNA known to be upregulated in liver and/or spleen may be used to enhance expression of a nucleic acid molecule, such as a polynucleotides, in liver and/or spleen.

In embodiments, the 5′ UTR sequence is derived from human hemoglobin subunit beta, which has the following nucleotide sequence:

(SEQ ID NO: 93)
ACATTTGCTTCTGACACAACTGTGTTCACTAGCAACCTCAAACAGACACC.

In embodiments, the 5′UTR comprises a nucleotide acid sequence that is at least about 90%, or at least about 92%, or at least about 94%, or at least about 95%, or at least about 98%, or about 100% identical to the nucleotide acid sequence of SEQ ID NO: 93. In embodiments, the 5′UTR comprises a nucleotide acid sequence that is at least about 90%, or at least about 92%, or at least about 94%, or at least about 95%, or at least about 98%, or about 100% identical to the nucleotide acid sequence of SEQ ID NO: 93 with one or more T nucleotide replaced with a U nucleotide, a modified U nucleotide or a homolog thereof. In embodiments, the 5′UTR comprises a nucleotide acid sequence that is at least about 90%, or at least about 92%, or at least about 94%, or at least about 95%, or at least about 98%, or about 100% identical to the nucleotide acid sequence of SEQ ID NO: 93 with at least 1, at least 2, at least 3, at least 4, at least 5, at least 6, at least 7, at least 8, at least 9, at least 10, at least 11, or all 12 T nucleotide replaced with a U nucleotide, a modified U nucleotide or a homolog thereof. In embodiments, the 5′UTR comprises a nucleotide acid sequence that is at least about 90%, or at least about 92%, or at least about 94%, or at least about 95%, or at least about 98%, or about 100% identical to the nucleotide acid sequence of SEQ ID NO: 93 wherein at least 1, at least 2, at least 3, at least 4, at least 5, at least 6, at least 7, at least 8, at least 9, at least 10, at least 11, or all 12 of the thymidine residues present in SEQ ID NO: 93 are replaced with modified uridine residues; at least 1, at least 2, at least 3, at least 4, at least 5, at least 6, at least 7, at least 8, at least 9, at least 10, at least 11, at least 12, at least 13, at least 14, at least 15, or all 16 of the cytosine residues present in SEQ ID NO: 93 are replaced with modified cytidine residues; at least 1, at least 2, at least 3, at least 4, at least 5, at least 6, at least 7, at least 8, at least 9, at least 10, at least 11, at least 12, at least 13, at least 14, or all 15 of the adenosine residues present in SEQ ID NO: 93 are replaced with modified adenosine residues; and/or at least 1, at least 2, at least 3, at least 4, at least 5, or all 6 of the guanosine residues present in SEQ ID NO: 93 are replaced with modified guanosine residues.

In embodiments, the 5′ UTR sequence is derived from a hepatitis C virus UTR sequence, which has the following nucleotide sequence:

(SEQ ID NO: 94)
TTGGGGGCGACACTCCACCATAGATCACTCCCCTGTGAGGAACTACTGTCTTCACGCAGAAAGCGTCT
AGCCATGGCGTTAGTATGAGTGTCGTGCAGCCTCCAGGACCCCCCCTCCCGGGAGAGCCATAGTGGT
CTGCGGAACCGGTGAGTACACCGGAATTGCCAGGACGACCGGGTCCTTTCTTGGATTAACCCGCTCAA
TGCCTGGAGATTTGGGCGTGCCCCCGCGAGACTGCTAGCCGAGTAGTGTTGGGTCGCGAAAGGCCTT
GTGGTACTGCCTGATAGGGTGCTTGCGAGTGCCCCGGGAGGTCTCGTAGACCGTGCATCATGAGCAC
AAATCCTAAACCTCAAAGAAAAACCAAACGTAACAAGGGCGAATTCGTTGGTAAAGCCACC.

In embodiments, the 5′UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 94. In embodiments, the 5′UTR comprises a nucleotide acid sequence that is at least about 90%, or at least about 95%, or at least about 96%, or at least about 97%, or at least about 98%, or at least about 99%, or 100% identical to the nucleotide acid sequence of SEQ ID NO: 94 with one or more T nucleotide replaced with a U nucleotide, a modified U nucleotide or a homolog thereof. In embodiments, the 5′UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 94 with at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% T nucleotide replaced with a U nucleotide, a modified U nucleotide or a homolog thereof. In embodiments, the 5′UTR comprises a nucleotide acid sequence that is at least about 90%, or at least about 95%, or at least about 96%, or at least about 97%, or at least about 98%, or at least about 99%, or about 100% identical to the nucleotide acid sequence of SEQ ID NO: 94, wherein at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues, thymidine residues, adenosine residues and/or guanosine residues are replaced by modified cytidine residues, modified uridine residues, modified adenosine residues and/or modified guanosine residues, respectively.

In embodiments, the 5′ UTR sequence lacks an AUG sequence element. In embodiments, the 5′ UTR sequence contains a GC-rich region. In embodiments the GC rich region is at least 8 nucleotides in length. In embodiments the GC rich region is located immediately upstream of the Kozak sequence. In embodiments that GC rich region contains between 30% and 49% cytosine.

In embodiments, at least one of the regions of linked nucleosides of A comprises a sequence of linked nucleosides which functions as a 5′ UTR and at least one of the regions of linked nucleosides of C comprises a sequence of linked nucleosides which functions as a 3′ UTR. In embodiments, the 5′ UTR and the 3′ UTR are from the same or different species. In embodiments, the 5′ UTR and the 3′ UTR may be the native untranslated regions from different proteins from the same or different species. In embodiments, the 5′ UTR and the 3′ UTR may have synthetic sequences.

In embodiments, the mmRNA further comprises a 3′ polyadenylation (polyA tail).

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In embodiments, the polynucleotide is in vitro transcribed (IVT). In embodiments, the polynucleotide is chimeric. In embodiments, the polynucleotide is circular.

In embodiments, the polynucleotide is or comprises DNA. In embodiments, the polynucleotide is or comprises a minicircle or a plasmid DNA. In embodiments, the plasmid DNA is devoid of any prokaryotic components. In embodiments, the polynucleotide comprises a tissue-specific control element. In embodiments, the tissue-specific control element is a promoter or an enhancer. In embodiments, the plasmid DNA is an expression vector. In embodiments, the DNA is or comprises a minicircle. In embodiments, the minicircle is a circular molecule, which is optionally small. In embodiments, the minicircle utilizes a cellular transcription and translation machinery to produce an encoded gene product. In embodiments, the minicircle is devoid of any prokaryotic components. In embodiments, the minicircle only comprises substantially only sequences of mammalian origin (or those that have been optimized for mammalian cells). In embodiments, the minicircle lacks or has reduced amount of DNA sequence elements that are recognized by the innate immune system and/or toll-like receptors. In embodiments, the minicircle is produced by excising any bacterial components of from a parental plasmid, thereby making it smaller than a parental DNA sequence. In embodiments, the minicircle is of non-viral origin. In embodiments, the minicircle remains episomal. In embodiments, the minicircle does not replicate with a host cell. In embodiments, expression of the chimeric protein in non-dividing cells harboring a minicircle lasts for at least 2 days, or at least 4 days, or at least 6 days, or at least 8 days, or at least 10 days, or at least 12 days, or at least 14 days, or at least 16 days, or at least 18 days, or at least 20 days, or at least 22 days, or at least 24 days, or longer in dividing cells. In embodiments, expression of the chimeric protein in non-dividing cells harboring a minicircle lasts for at least 4 days, or at least 6 days, or at least 8 days, or at least 10 days, or at least 1 week, or at least 2 weeks, or at least 3 weeks, or at least 4 weeks, or at least 5 weeks, or at least 6 weeks, or at least 1 month, or at least 2 months, or at least 3 months, or at least 4 months, or at least 5 months, or at least 6 months, or at least 8 months, or longer in dividing cells.

In embodiments, the mmRNAs of the present disclosure are produced by means available in the art, including but not limited to in vitro transcription (IVT) and synthetic methods. Enzymatic IVT, solid-phase, liquid-phase, combined synthetic methods, small region synthesis, and ligation methods may be utilized. In embodiments, mmRNAs are made using IVT enzymatic synthesis methods. Methods of making polynucleotides by IVT are known in the art and are described in International Application PCT International Patent Publication No. WO 2013/151666, the contents of which are incorporated herein by reference in their entirety. Accordingly, the present disclosure also includes polynucleotides, e.g., DNA, constructs and vectors that may be used to in vitro transcribe an mRNA described herein.

In embodiments, the polynucleotide is DNA. In embodiments, the polynucleotide comprises a liver-specific control element. In embodiments, the liver-specific control element is a liver-specific promoter selected from albumin promoter, thyroxine-binding globulin (TBG) promoter, hybrid liver-specific promoter (HLP), human α1-antitrypsin promoter, LP1 promoter, and hemopexin promoter. The gene therapy in accordance with the present disclosure can be performed using vector systems. In embodiments, the liver-specific promoter is an LP1 promoter. The LP1 promoter can be a human LP1 promoter, which can be constructed as described, e.g., in Nathwani et al. Blood vol. 107(7) (2006):2653-61, which is incorporated herein by reference in its entirety.

In aspects, the present disclosure provides a vector comprising the polynucleotide of any one of the embodiments disclosed herein. In embodiments, the chimeric protein can be provided as an expression vector. In embodiments, the expression vector is a DNA expression vector or an RNA expression vector. In embodiments, the expression vector is a viral expression vector. In embodiments, the expression vector is a non-viral expression vector (without limitation, e.g., a plasmid).

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. In embodiments, the expression vector may include, among others, chromosomal and episomal vectors, e.g., vectors derived from bacterial plasmids, from transposons, from yeast episomes, from insertion elements, from yeast chromosomal elements, and vectors derived from combinations thereof. The present constructs may contain control regions that regulate as well as engender expression.

A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In embodiments, the expression vector is an autonomously replicating plasmid or a virus (e.g., AAV vectors). In embodiments, the expression vector is non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In embodiments, the polynucleotide or cell therapy may employ expression vectors, which comprise the nucleic acid encoding the chimeric protein operably linked to an expression control region that is functional in the host cell. The expression control region is capable of driving expression of the operably linked encoding nucleic acid such that the chimeric protein is produced in a human cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a human cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In various embodiments, the chimeric protein expression is inducible or repressible. Typically, the amount of increase or decrease conferred by such elements is proportional to the amount of signal present; the greater the amount of signal, the greater the increase or decrease in expression.

Expression systems functional in human cells are well known in the art and include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family, and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences including CRISPR/CAS9, zinc finger, TALEN, and meganuclease gene-editing technologies.

In aspects, the present disclosure provides a host cell comprising the vector of any of the embodiments disclosed herein. A host cell comprising the mmRNA of any of the embodiments disclosed herein.

### Pharmaceutical Compositions

In aspects, the present disclosure provides a pharmaceutical composition comprising the chimeric protein of any one of the embodiments disclosed herein, the isolated polynucleotide of any one of the embodiments disclosed herein, or the vector of the embodiments disclosed herein, the host cell of any of the embodiments disclosed herein, and a pharmaceutically acceptable carrier. In embodiments, the pharmaceutical composition comprises the mmRNA of any one of the embodiments disclosed herein.

Suitable pharmaceutical compositions are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO 2017112943, WO 2014/028429, WO 2017/201325 the entire contents of which are hereby incorporated by reference.

In aspects, the present disclosure relates to a pharmaceutical composition comprising an isolated modified mRNA (mmRNA) encoding a heterologous chimeric protein having an amino acid sequence that has at least about 95% sequence identity with an amino acid sequence selected from SEQ ID NOs: 80-93.

In embodiments, the carrier is mmRNA comprises a modification (e.g., an RNA element), wherein the modification provides a desired translational regulatory activity. Such modifications are described in PCT Application No. PCT International Application Publication No. WO 2018/213789, the entire contents of which are herein incorporated by reference.

In embodiments, the mmRNA further comprises a 3′ untranslated region (UTR). In embodiments, the 3′ UTR comprises at least one microRNA-122 (miR-122) binding site. In embodiments, the miR-122 binding site is a miR-122-3p binding site or a miR-122-5-binding site. In embodiments, the mmRNA further comprises a spacer sequence between the open reading frame and the miRNA binding site. In embodiments, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR harbors a Kozak sequence and/or forms a secondary structure that stimulate elongation factor binding.

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In any of the embodiments disclosed herein, the mmRNA may comprise one or more modifications. In any of the embodiments disclosed herein, the mmRNA may comprise at least one modification. In embodiments, the modification is nucleoside modification. In embodiments, the modification is a base modification. In embodiments, the modification is a sugar-phosphate backbone modification.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and a combination of any two or more thereof. In embodiments, the modifications are selected from pseudouridine (Y), N1-methylpseudouridine (m1Y), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), .alpha.-thio-guanosine, .alpha.-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1l), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof. In embodiments, modification is selected from pseudouridine, N1-methylpseudouridine, 5-methylcytosine, 5-methoxyuridine, and a combination thereof.

In embodiments, the mmRNA comprises at least one N1-methylpseudouridine. In embodiments, the mmRNA is fully modified with chemically-modified uridines. In embodiments, the mmRNA is a fully modified with N1-methylpseudouridine.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

In embodiments, the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, the modifications are selected from 2-aminopurine, 2,6-diaminopurine, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In embodiments, the modifications are present on the major groove face. In embodiments, a hydrogen on C-5 of uracil is replaced with a methyl group or a halo group.

In embodiments, the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

In embodiments, the pharmaceutical composition is formulated as a lipid nanoparticle (LNP), a lipoplex, or a liposome. In embodiments, the pharmaceutical composition is formulated as a lipid nanoparticle (LNP). In embodiments, the mmRNAs described herein may be formulated in a cationic oil-in-water emulsion where the emulsion particle comprises an oil core and a cationic lipid that can interact with the mRNA anchoring the molecule to the emulsion particle. In embodiments, the mRNAs described herein may be formulated in a water-in-oil emulsion comprising a continuous hydrophobic phase in which the hydrophilic phase is dispersed. Exemplary emulsions can be made by the methods described in PCT International Application Publication Nos. WO 2012/006380 and WO 2010/87791, each of which is herein incorporated by reference in its entirety.

In some embodiments, nucleic acids of the invention (e.g., mRNA) are formulated in a lipid nanoparticle (LNP). Lipid nanoparticles comprise typically comprise ionizable cationic lipid, non-cationic lipid, sterol and PEG lipid components along with the nucleic acid cargo of interest. The lipid nanoparticles of the invention can be generated using components, compositions, and methods as are disclosed, e.g., in PCT International Application Publication Nos. WO 2021/231854, WO 2021/050986, WO 2021/055833, WO 2021/213924, WO 2021/055849, WO 2021/214204, WO 2021/188969, WO 2021/055835, WO 2020/061284, WO 2020/061295, WO 2017/049245, WO 2017/031232, WO 2017/112865, WO 2017/218704, WO 2017/218704, WO 2017/099823, WO 2017/049074, WO 2017/117528, WO 2017/180917, WO 2017/075531, WO 2017/223135, WO 2016/118724, WO 2015/164674, WO 2015/038892, WO 2014/152211, and WO 2013/090648, the entire contents of each which are herein incorporated by reference. PEG-lipids selected from an ionizable lipid (e.g. as known in the art, such as those described in U.S. Pat. No. 8,158,601 and PCT International Application Publication Nos. WO 2012/099755 and WO 2015/130584, which are incorporated herein by reference in their entirety. The ionizable lipid may be selected from, but not limited to, a ionizable lipid described in International Publication Nos. WO 2013/086354 and WO 2013/116126; the contents of each of which are herein incorporated by reference in their entirety. In embodiments, the lipid may be a cleavable lipid such as those described in PCT International Publication No. WO 2012/170889, herein incorporated by reference in its entirety. In embodiments, the lipid may be synthesized by methods known in the art and/or as described in International Publication Nos. WO 2013/086354; the contents of each of which are herein incorporated by reference in their entirety. In embodiments, the LNP formulations described herein can additionally comprise a permeability enhancer molecule. Non-limiting permeability enhancer molecules are described in U.S. Publication No. US 2005/0222064, herein incorporated by reference in its entirety.

In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as lipid nanoparticles (LNPs), a lipoplex, or a liposome. In embodiments, the pharmaceutical composition is formulated as lipid nanoparticles (LNPs).

In embodiments, the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the polynucleotide (e.g., mmRNA of any of the embodiments disclosed herein).

In embodiments, the LNP comprises a molar ratio of about 20-60% ionizable amino lipid, about 5-25% phospholipid, about 25-55% structural lipid, and about 0.5-1.5% PEG lipid. In embodiments, the ionizable amino lipid comprises the following formula:

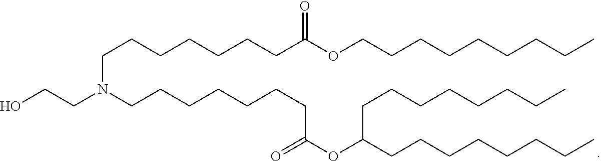 [SMILES: CCCCCCCCCOC(=O)CCCCCCCN(CCO)CCCCCCCC(=O)OC(CCCCCCCC)CCCCCCCC]

In embodiments, the lipid nanoparticles comprise lipids selected from one or more of an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the polynucleotide (e.g., mmRNA of any of the embodiments disclosed herein). In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g. C12, a PEG-dimyristyloxypropyl (C14), a PEG-dipalmityloxypropyl (C16), or a PEG-distearyloxypropyl (C18)).

In embodiments, the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the isolated polynucleotide is conjugated polynucleotide sequence that is introduced into cells by various transfection methods such as, e.g., methods that employ lipid particles. In embodiments, a composition, including a gene transfer construct, comprises a delivery particle. In embodiments, the delivery particle comprises a lipid-based particle (e.g., a lipid nanoparticle (LNP), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of small interfering RNA (siRNA) and mRNA, and for in vitro and in vivo delivering CRISPR/Cas9 components to hepatocytes and the liver. For example, U.S. Pat. No. 10,195,291 describes the use of LNPs for delivery of RNA interference (RNAi) therapeutic agents.

In embodiments, the composition in accordance with embodiments of the present disclosure is in the form of a LNP. In embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC).

In embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively. In embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5.

In embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-′)amino)ethyl)(2 hydroxydodecyl)amino)ethyl) piperazin-1-yl)ethylazanediyl)didodecan-2-ol (Tech G1), 1,2-Dilinoleyloxo-3-(2-N,N-dimethylamino) ethoxypropane (DLin-EG-DMA), or a mixture thereof.

In embodiments, the LNP comprises one or more molecules selected from polyethylenimine (PEI) and poly(lactic-co-glycolic acid) (PLGA), and N-Acetylgalactosamine (GalNAc), which are suitable for hepatic delivery. In embodiments, the LNP comprises a hepatic-directed compound as described, e.g., in U.S. Pat. No. 5,985,826, which is incorporated by reference herein in its entirety. GalNAc is known to target Asialoglycoprotein Receptor (ASGPR) expressed on mammalian hepatic cells. See Hu et al. Protein Pept Lett. 2014; 21(10):1025-30.

In some examples, the isolated polynucleotide can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, the LNP formulations may further contain a phosphate conjugate, which can increase in vivo circulation times and/or increase the targeted delivery of the nanoparticle. Phosphate conjugates can be made by the methods described in, e.g., PCT International Publication No. WO 2013033438 or U.S. Pub. No. US 20130196948. The LNP formulation can also contain a polymer conjugate (e.g., a water-soluble conjugate) as described in, e.g., U.S. Publication Nos. US 20130059360, US 20130196948, and US 20130072709, each of the references is herein incorporated by reference in its entirety.

In embodiments, the LNP formulations may comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., PCT International Publication No. WO 2012/109121, herein incorporated by reference in its entirety). In embodiments, the LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Publication No. US 2013/0183244, herein incorporated by reference in its entirety. In embodiments, the LNP formulations can be engineered to alter the surface properties of particles so that the lipid nanoparticles can penetrate the mucosal barrier as described in U.S. Pat. No. 8,241,670 or PCT International Publication No. WO 2013/110028, each of which is herein incorporated by reference in its entirety. In embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., PCT International Publication No. WO 2013/110028, herein incorporated by reference in its entirety.

In embodiments, an mmRNA described herein is formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in PCT International Publication No. WO 2013105101, herein incorporated by reference in its entirety.

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In embodiments, the chimeric protein or the therapeutic nanoparticle comprising mRNA can be formulated for sustained release, which, as used herein, refers to a pharmaceutical composition or compound that conforms to a release rate over a specific period of time. In embodiments, the period of time may include, but is not limited to, hours, days, weeks, months and years. As a non-limiting example, the sustained release nanoparticle of the mRNAs described herein can be formulated as disclosed in PCT International Publication No. WO 2010/075072 and U.S. Publication Nos. US 2010/0216804, US 2011/0217377, US 2012/0201859 and US 2013/0150295, each of which is herein incorporated by reference in their entirety.

In embodiments, the chimeric protein or the isolated polynucleotide (e.g., mmRNA) (and/or additional agents) are included various formulations. Any chimeric protein, or the isolated polynucleotide (e.g., mmRNA) (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In embodiments, the present disclosure provides an expression vector, comprising a nucleic acid encoding the chimeric protein described herein. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λP_{L}. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the disclosure comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the disclosure is capable of expressing operably linked encoding nucleic acid in a human cell. In embodiments, the cell is a tumor cell. In embodiments, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present disclosure contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter which is usually located 5′ of the transcribed sequence. Another example of an expression control element is an enhancer, which can be located 5′ or 3′ of the transcribed sequence, or within the transcribed sequence.

Expression systems functional in human cells are well known in the art and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric proteins including CRISPR/CAS9, zinc finger, TALEN, and meganuclease gene-editing technologies.

In aspects, the disclosure provides expression vectors for the expression of the chimeric proteins (and/or additional agents) that are viral vectors. Many viral vectors useful for gene therapy are known (see, e.g., Lundstrom, Trends Biotechnol., 21:1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the disclosure provides methods of transducing a human cell in vivo, comprising contacting a solid tumor in vivo with a viral vector of the disclosure.

In embodiments, the present disclosure provides a host cell, comprising the expression vector comprising the chimeric protein described herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

Where necessary, the formulations comprising the chimeric protein, or the isolated polynucleotide (e.g., mmRNA) (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the chimeric protein (and/or additional agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any chimeric protein, or the isolated polynucleotide (e.g., mmRNA) (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In some instances, administration results in the release of any agent described herein into the bloodstream, or alternatively, the agent is administered directly to the site of active disease.

Any chimeric protein, or the isolated polynucleotide (e.g., mmRNA) (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In embodiments, the pharmaceutical composition is formulated for parenteral administration. In embodiments, the pharmaceutical composition is formulated for intradermal, intramuscular, intraperitoneal, intraarticular, intravenous, subcutaneous, intraarterial or transdermal administration.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein, or the isolated polynucleotide (e.g., mmRNA) (and/or additional agents) described herein as well as the dosing schedule can depend on various parameters, including, but not limited to, the disease being treated, the subject's general health, and the administering physician's discretion. Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart.

The dosage of any chimeric protein, or the isolated polynucleotide (e.g., mmRNA) (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In embodiments, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any chimeric protein, or the isolated polynucleotide (e.g., mmRNA) (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533) may be used.

Administration of any chimeric protein, or the isolated polynucleotide (e.g., mmRNA) (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein, or the isolated polynucleotide (e.g., mmRNA) (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any chimeric protein (and/or additional agents) described herein can be administered in a single daily dose, or the total daily dosage can be administered in divided doses of two, three or four times daily. Furthermore, any chimeric protein (and/or additional agents) described herein can be administered continuously rather than intermittently throughout the dosage regimen.

### Methods of Treatment

In aspects, the present disclosure provides a method of treating nonalcoholic fatty liver disease (NAFLD) or non-alcoholic steatohepatitis (NASH) in a subject in need thereof, the method comprising administering pharmaceutical composition comprising a polynucleotide any one of the embodiments disclosed herein encoding a chimeric protein of any one of the embodiments disclosed herein.

NAFLD is characterized by hepatic steatosis with no secondary causes of hepatic steatosis including excessive alcohol consumption, other known liver diseases, or long-term use of a steatogenic medication (Chalasani et al., The diagnosis and management of nonalcoholic fatty liver disease: Practice guidance from the American Association for the Study of Liver Diseases, Hepatology 2018, 67(1):328-357, which is hereby incorporated by reference in its entirety). In embodiments, the subject has NAFLD selected from non-alcoholic fatty liver (NAFL) and non-alcoholic steatohepatitis (NASH). In embodiments, the subject has NAFL, as indicated by the presence of >5% hepatic steatosis without evidence of hepatocellular injury in the form of hepatocyte ballooning. In embodiments, the subject has NASH as indicated by the presence of >5% hepatic steatosis and inflammation with hepatocyte injury (e.g., ballooning), with or without any liver fibrosis. In embodiments, the subject has NASH, which is associated with hepatic inflammation and liver fibrosis, which optionally has progressed to cirrhosis, end-stage liver disease, and/or hepatocellular carcinoma. In embodiments, the subject has NASH without liver fibrosis. In embodiments, the subject has fibrosis of very low severity of fibrosis.

There are many approaches used to assess and evaluate whether a subject has NAFLD and if so, the severity of the disease including differentiating whether the NAFLD is NAFL or NASH. For example, these approaches include determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. Accordingly, in embodiments, the subject selected for the treatment with the compositions comprising the chimeric protein disclosed herein or the polynucleotides encoding the chimeric protein disclosed herein based on diagnosis by determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. In embodiments, the subject selected for the treatment with the chimeric protein and/or compositions disclosed herein based on diagnosis of a physiological indicator of NAFLD selected from liver morphology, liver stiffness, and the size or weight of the subject's liver. In some embodiments, NAFLD in the subject is evidenced by an accumulation of hepatic fat and detection of a biomarker indicative of liver damage. For example, elevated serum ferritin and low titers of serum autoantibodies can be common features of NAFLD. In embodiments, the subject selected for the treatment with the chimeric proteins or compositions disclosed herein based on diagnosis of NAFLD using a technique including, but not limited to, magnetic resonance imaging, either by spectroscopy or by proton density fat fraction (MRI-PDFF) to quantify steatosis, transient elastography (FIBROSCAN®), hepatic venous pressure gradient (HPVG), hepatic stiffness measurement with MRE for diagnosing significant liver fibrosis and/or cirrhosis, and assessing histological features of liver biopsy. In some embodiments, magnetic resonance imaging is used to detect one or more of steatohepatitis (NASH-MRI), liver fibrosis (Fibro-MRI), and steatosis see, for example, U.S. Application Publication Nos. 2016/146715 and 2005/0215882, each of which are incorporated herein by reference in their entireties.

In embodiments, the subject selected for the treatment with the compositions comprising the chimeric protein disclosed herein or the polynucleotides encoding the chimeric protein disclosed herein based on diagnosis symptoms selected from one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In embodiments, hepatic steatosis is determined by one or more methods selected from ultrasonography, computed tomography (CT), magnetic resonance imaging, magnetic resonance spectroscopy (MRS), magnetic resonance elastography (MRE), transient elastography (TE) (e.g., FIBROSCAN®), measurement of liver size or weight, or by liver biopsy (see, e.g., Di Lascio et al, Ultrasound Med Biol. 2018; 44(8): 1585-1596; Lv et al, J Clin Transl Hepatol. 2018 Jun. 28; 6(2): 217-221; Reeder, et ah, JMagn Re son Imaging. 2011 October; 34(4): 848-855; and de Ledinghen V, et ah, J Gastroenterol Hepatol. 2016 April; 31(4):848-55, each of which are incorporated herein by reference in their entireties). In embodiments, a subject diagnosed with NAFLD may have more than about 5% hepatic steatosis, for example, about 5% to about 25%, about 25% to about 45%, about 45% to about 65%, or greater than about 65% hepatic steatosis. In embodiments, a subject with about 5% to about 33% hepatic steatosis has stage 1 hepatic steatosis, a subject with about 33% to about 66% hepatic steatosis has stage 2 hepatic steatosis, and a subject with greater than about 66% hepatic steatosis has stage 3 hepatic steatosis.

In embodiments, the amount of hepatic steatosis is determined prior to administration of the combination of (a) the compositions comprising the chimeric protein disclosed herein or the polynucleotides encoding the chimeric protein disclosed herein, and (b) an additional therapeutic agent. In embodiments, the amount of hepatic steatosis is determined during the period of time or after the period of time of administration of the combination of (a) and (b). In embodiments, a reduction in the amount of hepatic steatosis during the period of time or after the period of time of administration of the combination of (a) and (b) compared to prior to administration of the combination of (a) and (b) indicates treatment of NAFLD. For example, a reduction in the amount of hepatic steatosis by about 1% to about 50%, about 25% to about 75%, or about 50% to about 100% indicates treatment of NAFLD. In embodiments, a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In embodiments, treatment of NAFLD can be assessed by measuring hepatic steatosis. In embodiments, treatment of NAFLD comprises a reduction in hepatic steatosis following administration of one or more chimeric proteins or composition encoding the chimeric proteins described herein. In embodiments, the treatment of NAFLD with the chimeric proteins or compositions disclosed herein comprises one or more of a decrease in symptoms; a reduction in the amount of hepatic steatosis; a decrease in the NAS; a decrease in hepatic inflammation; a decrease in the level of biomarkers indicative of one or more of liver damage, inflammation, liver fibrosis, and/or liver cirrhosis; and a reduction in fibrosis and/or cirrhosis, a lack of further progression of fibrosis and/or cirrhosis, or a slowing of the progression of fibrosis and/or cirrhosis. In embodiments, treatment of NAFLD comprises a decrease of one or more symptoms associated with NAFLD in the subject. In embodiments, the total body weight of the subject does not increase. In embodiments, the total body weight of the subject decreases. In embodiments, the body mass index (BMI) of the subject does not increase. In embodiments, the body mass index (BMI) of the subject decreases. In embodiments, the waist and hip (WTH) ratio of the subject does not increase. In embodiments, the waist and hip (WTH) ratio of the subject decreases.

In embodiments, the severity of NALFD can be assessed using the NAS. In embodiments, treatment of NAFLD can be assessed using the NAS. In embodiments, treatment of NAFLD comprises a reduction in the NAS following administration of one or more compounds described herein. In embodiments, the NAS can be determined as described in Kleiner et al., Hepatology. 2005, 41(6): 1313-1321, which is hereby incorporated by reference in its entirety.

In embodiments, the NAS following administration is determined non-invasively, for example, as described in U.S. Application Publication No. 2018/0140219, which is incorporated by reference herein in its entirety. In embodiments, the NAS following administration is determined for a sample from the subject prior to administration of the compositions comprising the chimeric protein disclosed herein or the polynucleotides encoding the chimeric protein disclosed herein. In embodiments, the NAS following administration is determined during the period of time or after the period of time of administration of the compositions comprising the chimeric protein disclosed herein or the polynucleotides encoding the chimeric protein disclosed herein. In embodiments, a lower NAS score during the period of time or after the period of time of administration of the compositions comprising the chimeric protein disclosed herein or the polynucleotides encoding the chimeric protein disclosed herein compared to prior to administration of the compositions comprising the chimeric protein disclosed herein or the polynucleotides encoding the chimeric protein disclosed herein indicates treatment of NAFLD. For example, a decrease in the NAS by 1, by 2, by 3, by 4, by 5, by 6, or by 7 indicates treatment of NAFLD. In embodiments, the NAS following administration of the compositions comprising the chimeric protein disclosed herein or the polynucleotides encoding the chimeric protein disclosed herein is 7 or less. In embodiments, the NAS during the period of time of administration of the compositions comprising the chimeric protein disclosed herein or the polynucleotides encoding the chimeric protein disclosed herein is 5 or less, 4 or less, 3 or less, or 2 or less. In embodiments, the NAS during the period of time of administration of the compositions comprising the chimeric protein disclosed herein or the polynucleotides encoding the chimeric protein disclosed herein is 7 or less. In embodiments, the NAS during the period of time of administration of the compositions comprising the chimeric protein disclosed herein or the polynucleotides encoding the chimeric protein disclosed herein is 5 or less, 4 or less, 3 or less, or 2 or less. In embodiments, the NAS after the period of time of administration of the compositions comprising the chimeric protein disclosed herein or the polynucleotides encoding the chimeric protein disclosed herein is 7 or less. In embodiments, the NAS after the period of time of administration of the compositions comprising the chimeric protein disclosed herein or the polynucleotides encoding the chimeric protein disclosed herein is 5 or less, 4 or less, 3 or less, or 2 or less.

In some embodiments, the presence of hepatic inflammation is determined by one or more methods selected from the group consisting of biomarkers indicative of hepatic inflammation and a liver biopsy sample(s) from the subject. In some embodiments, the severity of hepatic inflammation is determined from a liver biopsy sample(s) from the subject. For example, hepatic inflammation in a liver biopsy sample can be assessed as described in Kleiner et al., Hepatology. 2005, 41(6): 1313-1321 and Brunt et al., Am J Gastroenterol 1999, 94:2467-2474, each of which are hereby incorporated by reference in their entireties.

In some embodiments, the severity of hepatic inflammation is determined prior to administration of the compositions comprising the chimeric protein disclosed herein or the polynucleotides encoding the chimeric protein disclosed herein. In some embodiments, the severity of hepatic inflammation is determined prior to administration of the compositions comprising the chimeric protein disclosed herein or the polynucleotides encoding the chimeric protein disclosed herein. In some embodiments, the severity of hepatic inflammation is determined during the period of time or after the period of time of administration of the compositions comprising the chimeric protein disclosed herein or the polynucleotides encoding the chimeric protein disclosed herein. In some embodiments, a decrease in the severity of hepatic inflammation during the period of time or after the period of time of administration of the compositions comprising the chimeric protein disclosed herein or the polynucleotides encoding the chimeric protein disclosed herein compared to prior to administration of the compositions comprising the chimeric protein disclosed herein or the polynucleotides encoding the chimeric protein disclosed herein indicates treatment of NAFLD. For example, a decrease in the severity of hepatic inflammation by about 1% to about 50%, about 25% to about 75%, or about 50% to about 100% indicates treatment of NAFLD. In some embodiments, a decrease in the severity of hepatic inflammation by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, treatment of NAFLD comprises treatment of fibrosis and/or cirrhosis, e.g., a decrease in the severity of fibrosis, a lack of further progression of fibrosis and/or cirrhosis, or a slowing of the progression of fibrosis and/or cirrhosis. In some embodiments, the presence of fibrosis and/or cirrhosis is determined by one or more methods selected from the group consisting of transient elastography (e.g., FIBROSCAN®), non-invasive markers of hepatic fibrosis, and histological features of a liver biopsy. In some embodiments, the severity (e.g., stage) of fibrosis is determined by one or more methods selected from the group consisting of transient elastography (e.g., FIBROSCAN®), a fibrosis-scoring system, biomarkers of hepatic fibrosis (e.g., non-invasive biomarkers), and hepatic venous pressure gradient (HVPG). Non-limiting examples of fibrosis scoring systems include the NAFLD fibrosis scoring system (see, e.g., Angulo, et al., Hepatology. 2007; 45(4):846-54), the fibrosis scoring system in Brunt et al., Am J Gastroenterol. 1999, 94:2467-2474, the fibrosis scoring system in Kleiner et al., Hepatology. 2005, 41(6): 1313-1321, and the ISHAK fibrosis scoring system (see Ishak et al., J Hepatol. 1995; 22:696-9), the contents of each of which are incorporated by reference herein in their entireties.

In some embodiments, the severity of fibrosis is determined prior to administration of the compositions comprising the chimeric protein disclosed herein or the polynucleotides encoding the chimeric protein disclosed herein. In some embodiments, the severity of fibrosis is determined prior to administration of a combination of (a) the compositions comprising the chimeric protein disclosed herein or the polynucleotides encoding the chimeric protein disclosed herein, and (b) an additional therapeutic agent. In some embodiments, the severity of fibrosis is determined during the period of time or after the period of time of administration of the combination of (a) and (b). In some embodiments, a decrease in the severity of fibrosis during the period of time or after the period of time of administration of the combination of (a) and (b) compared to prior to administration of the combination of (a) and (b) indicates treatment of NAFLD. In some embodiments, a decrease in the severity of fibrosis, a lack of further progression of fibrosis and/or cirrhosis, or a slowing of the progression of fibrosis and/or cirrhosis indicates treatment of NAFLD. In some embodiments, the severity of fibrosis is determined using a scoring system such as any of the fibrosis scoring systems described herein, for example, the score can indicate the stage of fibrosis, e.g., stage 0 (no fibrosis), stage 1, stage 2, stage 3, and stage 4 (cirrhosis) (see, e.g., Kleiner et al). In some embodiments, a decrease in the stage of the fibrosis is a decrease in the severity of the fibrosis. For example, a decrease by 1, 2, 3, or 4 stages is a decrease in the severity of the fibrosis. In some embodiments, a decrease in the stage, e.g., from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 indicates treatment of NAFLD. In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 following administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 during the period of time of administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 after the period of time of administration of the combination of (a) and (b) compared to prior to administration of the combination of (a) and (b).

In some embodiments, the presence of NAFLD is determined by one or more biomarkers indicative of one or more of liver damage, inflammation, liver fibrosis, and/or liver cirrhosis or scoring systems thereof. In some embodiments, the severity of NAFLD is determined by one or more biomarkers indicative of one or more of liver damage, inflammation, liver fibrosis, and/or liver cirrhosis or scoring systems thereof. The level of the biomarker can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et ah, Gut. 2010 September; 59(9): 1265-9, which is incorporated by reference herein in its entirety); hyaluronic acid; pro-inflammatory cytokines; a panel of biomarkers consisting of a2-macroglobulin, haptoglobin, apolipoprotein AI, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, a2-macroglobulin combined with the subject's age and sex (e.g., HEPASCORE®; see, e.g., Adams et al., Clin Chem. 2005, 51(10): 1867-73), and a panel of biomarkers consisting of tissue inhibitor of metalloproteinase-1, hyaluronic acid, and a2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 August; 59(2): 236-42, which is incorporated by reference herein in its entirety). In some embodiments, the presence of fibrosis is determined by one or more of the FIB-4 score, a panel of biomarkers consisting of a2-macroglobulin, haptoglobin, apolipoprotein AI, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, a2-macroglobulin combined with the subject's age and sex (e.g., HEPASCORE®; see, e.g., Adams et al., Clin Chem. 2005 October; 51(10): 1867-73), and a panel of biomarkers consisting of tissue inhibitor of metalloproteinase-1, hyaluronic acid, and a2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, the severity of fibrosis is determined by one or more of the FIB-4 score, a panel of biomarkers consisting of a2-macroglobulin, haptoglobin, apolipoprotein AI, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, a2-macroglobulin combined with the subject's age and sex (e.g., HEPASCORE®; see, e.g., Adams et al., Clin Chem. 2005 October; 51(10): 1867-73, which is incorporated by reference herein in its entirety), and a panel of biomarkers consisting of tissue inhibitor of metalloproteinase-1, hyaluronic acid, and a2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score). In some embodiments, hepatic inflammation is determined by the level of liver inflammation biomarkers, e.g., pro-inflammatory cytokines. Non-limiting examples of biomarkers indicative of liver inflammation include interleukin-(IL) 6, interleukin-(IL) 1b, tumor necrosis factor (TNF)-a, transforming growth factor (TGF)-P, monocyte chemotactic protein (MCP)-I, C-reactive protein (CRP), PAI-1, and collagen isoforms such as CollaI, Colla2, and Col4aI (see, e.g., Neuman, et ah, Can J Gastroenterol Hepatol. 2014 December; 28(11): 607-618 and U.S. Pat. No. 9,872,844, each of which are incorporated by reference herein in their entireties). Liver inflammation can also be assessed by change of macrophage infiltration, e.g., measuring a change of CD68 expression level. In some embodiments, liver inflammation can be determined by measuring or monitoring serum levels or circulating levels of one or more of interleukin-(IL) 6, interleukin-(IL) 1b, tumor necrosis factor (TNF)-a, transforming growth factor (TGF-b, monocyte chemotactic protein (MCP)-I, and C-reactive protein (CRP).

In some embodiments, the level of one or more biomarkers indicative of one or more of liver damage, inflammation, liver fibrosis, and/or liver cirrhosis is determined for a sample from the subject prior to administration of the combination of (a) the compositions comprising the chimeric protein disclosed herein or the polynucleotides encoding the chimeric protein disclosed herein, and (b) an additional therapeutic agent. In some embodiments, the level of one or more biomarkers indicative of one or more of liver damage, inflammation, liver fibrosis, and/or liver cirrhosis is determined during the period of time or after the period of time of administration of the combination of (a) and (b). In some embodiments, a decrease in the level of one or more biomarkers indicative of one or more of liver damage, inflammation, liver fibrosis, and/or liver cirrhosis during the period of time or after the period of time of administration of the combination of (a) and (b) compared to prior to administration of the combination of (a) and (b) indicates treatment of NAFLD. For example, a decrease in the level of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of NAFLD. In some embodiments, the decrease in the level of one or more biomarkers indicative of one or more of liver damage, inflammation, liver fibrosis, and/or liver cirrhosis following administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis during the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis after the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the level of serum bile acids is determined by, for example, an ELISA enzymatic assay or the assays for the measurement of total bile acids as described in Danese et ah, PLOS One. 2017; 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of (a) and (b). In some embodiments, the NAFLD is NAFLD with attendant cholestasis. In cholestasis, the release of bile, including bile acids, from the liver is blocked. Bile acids can cause hepatocyte damage (see, e.g., Perez M J, Briz O. World J Gastroenterol. 2009 Apr. 14; 15(14): 1677-89) likely leading to or increasing the progression of fibrosis (e.g., cirrhosis) and increasing the risk of hepatocellular carcinoma (see, e.g., Sorrentino P et ah. Dig Dis Sci. 2005 June; 50(6): 1130-5 and Satapathy S K and Sanyal A J. Semin Liver Dis. 2015, 35(3):221-35, each of which are incorporated by reference herein in their entireties). In some embodiments, the NAFLD with attendant cholestasis is NASH with attendant cholestasis. In some embodiments, the treatment of NAFLD comprises treatment of pruritus. In some embodiments, the treatment of NAFLD with attendant cholestasis comprises treatment of pruritus. In some embodiments, a subject with NAFLD with attendant cholestasis has pruritus.

In some embodiments, treatment of NAFLD comprises an increase in adiponectin. It is thought that the compound of Formula (I) may be a selective activator of a highly limited number of PPARy pathways including pathways regulated by adiponectin. Adiponectin is an anti-fibrotic and anti-inflammatory adipokine in the liver (see e.g., Park et ah, Curr Pathobiol Rep. 2015 Dec. 1; 3(4): 243-252.). In some embodiments, the level of adiponectin is determined by, for example, an ELIS A enzymatic assay. In some embodiments, the adiponectin level in the subject is increased by at least about 30%, at least about 68%, at least about 175%, or at least about 200%. In some embodiments, the increase is by at least about 175%. In some embodiments, the level of adiponectin is determined for a sample from the subject prior to administration of the combination of (a) the compositions comprising the chimeric protein disclosed herein or the polynucleotides encoding the chimeric protein disclosed herein, and (b) an additional therapeutic agent. In some embodiments, the level of adiponectin is determined for a sample from the subject prior to administration of the combination of (a) the compositions comprising the chimeric protein disclosed herein or the polynucleotides encoding the chimeric protein disclosed herein, and (b) an additional therapeutic agent. In some embodiments, the level of adiponectin is determined during the period of time or after the period of time of administration of the combination of (a) and (b). In some embodiments, an increase in the level of adiponectin during the period of time or after the period of time of administration of the combination of (a) and (b) compared to prior to administration of the combination of (a) and (b) indicates treatment of NAFLD. For example, an increase in the level of adiponectin by at least about 30%, at least about 68%, at least about 175%, or at least about 200% indicates treatment of NAFLD. In some embodiments, the increase in the level of adiponectin following administration of the combination of (a) and (b) is at least about 200%.

Provided herein are methods of treating non-alcoholic fatty liver disease (NAFLD) in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating NAFLD. In some embodiments, a method of treating non-alcoholic fatty liver disease (NAFLD) in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating NAFLD.

Provided herein are methods of treating obesity in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating obesity. In some embodiments, a method of treating obesity in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating obesity.

Provided herein are methods of controlling body weight in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in controlling body weight. In some embodiments, a method of controlling body weight in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in controlling body weight.

Provided herein are methods of treating obesity in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating obesity. In some embodiments, a method of treating obesity in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating obesity.

Provided herein are methods of controlling plasma insulin in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in controlling plasma insulin. In some embodiments, a method of controlling plasma insulin in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in controlling plasma insulin.

Provided herein are methods of controlling food intake in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in controlling food intake. In some embodiments, a method of controlling food intake in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in controlling food intake.

Provided herein are methods of treating hyperglycemia in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating hyperglycemia. In some embodiments, a method of treating hyperglycemia in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating hyperglycemia.

Provided herein are methods of treating insulin resistance in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating insulin resistance. In some embodiments, a method of treating insulin resistance in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating insulin resistance.

Also provided herein are methods of treating fibrosis in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating fibrosis. In some embodiments, a method of treating fibrosis in a subject in need thereof comprises or consists essentially of administering to the subject (the compositions comprising the chimeric protein disclosed herein or the polynucleotides encoding the chimeric protein disclosed herein, during a period of time, wherein the amount of the compositions comprising the chimeric protein disclosed herein or the polynucleotides encoding the chimeric protein disclosed herein is effective in treating fibrosis.

Also provided herein are methods of treating steatosis in a subject in need thereof comprising or consisting essentially of administering to the subject the compositions comprising the chimeric protein disclosed herein or the polynucleotides encoding the chimeric protein disclosed herein, wherein the amount of the compositions comprising the chimeric protein disclosed herein or the polynucleotides encoding the chimeric protein disclosed herein is effective in treating steatosis. In some embodiments, a method of treating steatosis in a subject in need thereof comprises or consists essentially of administering to the subject the compositions comprising the chimeric protein disclosed herein or the polynucleotides encoding the chimeric protein disclosed herein during a period of time, wherein the amount of the compositions comprising the chimeric protein disclosed herein or the polynucleotides encoding the chimeric protein disclosed herein is effective in treating steatosis.

Also provided herein are methods of treating a subject, the method comprising: selecting a subject having non-alcoholic fatty liver disease (NAFLD); and administering the compositions comprising the chimeric protein disclosed herein or the polynucleotides encoding the chimeric protein disclosed herein to the selected subject, wherein the amount of (the compositions comprising the chimeric protein disclosed herein or the polynucleotides encoding the chimeric protein disclosed herein is effective in treating NAFLD. In some embodiments, the compositions comprising the chimeric protein disclosed herein or the polynucleotides encoding the chimeric protein disclosed herein is administered during a period of time. Also provided herein are methods of treating a subject, the method comprising: identifying a subject having non-alcoholic fatty liver disease (NAFLD); and administering the compositions comprising the chimeric protein disclosed herein or the polynucleotides encoding the chimeric protein disclosed herein to the selected subject, wherein the amount of the compositions comprising the chimeric protein disclosed herein or the polynucleotides encoding the chimeric protein disclosed herein is effective in treating NAFLD. In some embodiments, the compositions comprising the chimeric protein disclosed herein or the polynucleotides encoding the chimeric protein disclosed herein is administered during a period of time.

Also provided herein are methods of selecting a subject for participation in a clinical trial, the method comprising: identifying a subject having NAFLD; and selecting the identified subject for participation in a clinical trial that comprises administration of the compositions comprising the chimeric protein disclosed herein or the polynucleotides encoding the chimeric protein disclosed herein. In some embodiments, the amount of the compositions comprising the chimeric protein disclosed herein or the polynucleotides encoding the chimeric protein disclosed herein is effective in treating NAFLD.

In aspects, the present disclosure provides a method of treating or preventing non-alcoholic steatohepatitis (NASH) in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing nonalcoholic fatty liver disease (NAFLD) in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing obesity in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing metabolic syndrome in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing blood glucose in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing steady state plasma insulin in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing food intake in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing serum cholesterol in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver adiposity in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver weight in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing epididymal white adipose tissue (eWAT) accumulation in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing subcutaneous white adipose tissue (sWAT) accumulation in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of decreasing hepatocellular ballooning in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver fibrosis in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing excessive fatty acid intake by liver cells in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of protecting liver cells from fatty acid-induced toxicity in a subject in need thereof, the method comprising administering pharmaceutical composition comprising a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating nonalcoholic fatty liver disease (NAFLD) or non-alcoholic steatohepatitis (NASH) in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of treating or preventing non-alcoholic steatohepatitis (NASH) in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of treating or preventing nonalcoholic fatty liver disease (NAFLD) in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of treating or preventing obesity in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of treating or preventing metabolic syndrome in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing blood glucose in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing steady state plasma insulin in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing food intake in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing serum cholesterol in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing liver adiposity in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing liver weight in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing epididymal white adipose tissue (eWAT) accumulation in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing subcutaneous white adipose tissue (sWAT) accumulation in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of decreasing hepatocellular ballooning in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing liver fibrosis in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of reducing excessive fatty acid intake by liver cells in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In one aspect, the present disclosure provides a method of protecting liver cells from fatty acid-induced toxicity in a subject in need thereof, the method comprising administering pharmaceutical composition comprising a polynucleotide encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: N terminus—(a)-(b)-(c)—C terminus, wherein: (A) (a) is a first domain comprising glucagon-like peptide-1 (GLP-1), (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor 19 (FGF19); or (B) (a) is a first domain comprising a fibroblast growth factor 19 (FGF19), (b) is a linker adjoining the first domain and a second domain, optionally wherein the linker comprises one or more protease-cleavable polypeptide linkers, and/or a hinge-CH2-CH3 Fc domain, and (c) is the second domain comprising glucagon-like peptide-1 (GLP-1).

In aspects, the present disclosure provides a method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: N terminus—(a)-(b)-(c)—C terminus, wherein: (A) (a) is a first domain comprising glucagon-like peptide-1 (GLP-1), (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor 21 (FGF21); or (B) (a) is a first domain comprising a fibroblast growth factor 21 (FGF21), (b) is a linker adjoining the first domain and a second domain, optionally wherein the linker comprises one or more protease-cleavable polypeptide linkers, and/or a hinge-CH2-CH3 Fc domain, and (c) is the second domain comprising glucagon-like peptide-1 (GLP-1).

In aspects, the present disclosure provides a method of treating or preventing nonalcoholic fatty liver disease (NAFLD), non-alcoholic steatohepatitis (NASH), obesity, or metabolic syndrome, or of reducing blood glucose, or of reducing fed and fasting blood glucose, or of reducing steady state plasma insulin, or of reducing food intake, or of reducing serum cholesterol, or of reducing liver adiposity, or of reducing liver weight, or of reducing epididymal white adipose tissue (eWAT) accumulation, or of reducing subcutaneous white adipose tissue (sWAT) accumulation, or of decreasing hepatocellular ballooning, or of reducing liver fibrosis, or of reducing excessive fatty acid intake by liver cells, or of protecting liver cells from fatty acid-induced toxicity a subject in need thereof, the method comprising administering to the subject a chimeric protein of any one of the embodiments disclosed herein, the isolated polynucleotide of any one of the embodiments disclosed herein, or the pharmaceutical composition of any one of the embodiments disclosed herein, or the host cell of any of the embodiments disclosed herein.

In aspects, the present disclosure provides a method of treating or preventing non-alcoholic steatohepatitis (NASH) in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing nonalcoholic fatty liver disease (NAFLD) in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing obesity in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating or preventing metabolic syndrome in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing blood glucose in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing steady state plasma insulin in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing food intake in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing serum cholesterol in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver adiposity in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver weight in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing epididymal white adipose tissue (eWAT) accumulation in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing subcutaneous white adipose tissue (sWAT) accumulation in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of decreasing hepatocellular ballooning in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing liver fibrosis in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of reducing excessive fatty acid intake by liver cells in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of protecting liver cells from fatty acid-induced toxicity in a subject in need thereof, the method comprising administering pharmaceutical composition comprising a modified mRNA (mmRNA) encoding a chimeric protein having a general structure of: N terminus—(a)-(b)-(c)—C terminus, wherein: (A) (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or (B) (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In aspects, the present disclosure provides a method of treating nonalcoholic fatty liver disease (NAFLD) or non-alcoholic steatohepatitis (NASH) in a subject in need thereof, the method comprising administering pharmaceutical composition comprising a polynucleotide encoding a chimeric protein having a general structure of: N terminus—(a)-(b)-(c)—C terminus, wherein: A. (a) is a first domain comprising a glucagon-like peptide-1 (GLP-1) receptor agonist, (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor selected from fibroblast growth factor 19 (FGF19), FGF21, FGF23, a variant thereof, and an analog thereof; or B. (a) is a first domain comprising a fibroblast growth factor selected from fibroblast growth factor 19 (FGF19), FGF21, FGF23, a variant thereof, and an analog thereof, (b) is a linker adjoining the first domain and a second domain, optionally wherein the linker comprises one or more protease-cleavable polypeptide linkers, and/or a hinge-CH2-CH3 Fc domain, and (c) is the second domain comprising a glucagon-like peptide-1 (GLP-1) receptor agonist.

In embodiments, the GLP-1 receptor agonist is selected from wherein the GLP-1 receptor agonist is selected from GLP-1, DPP4 degradation resistant GLP-1 (7-37, A8G), exenatide, lixisenatide, albiglutide, dulaglutide, or a variant thereof having one or more amino acid mutations, independently selected from substitutions, insertions, deletions, and truncations. In embodiments, the GLP-1 receptor agonist has an amino acid sequence of any one of SEQ ID NOs: 57 to 62, 75, and 87-90, or a variant having about 1, 2, 3, 4, 5, 6, 7, 8, 9, or 10 or more amino acid mutations with respect to an amino acid sequence selected from SEQ ID NOS: 57 to 62, 75, and 87-90. In embodiments, the mutations are independently selected from substitutions, insertions, deletions, and truncations. In embodiments, the GLP-1 receptor agonist is capable of binding a GLP-1 receptor. In embodiments, the GLP-1 receptor agonist is capable of stimulating insulin secretion and/or inhibiting glucagon secretion.

In embodiments, the fibroblast growth factor comprises FGF19, or an analog thereof. In embodiments, the analog of FGF19 is aldafermin (NGM282). In embodiments, fibroblast growth factor is capable of activating FGFR4, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence selected from SEQ ID NOs: 65 or 66.

Additionally, or alternatively, in embodiments, the fibroblast growth factor comprises FGF21, or an analog thereof. In embodiments, the analog of FGF21 is selected from efruxifermin, LY2405319, FGF21 (RGE) and FGF21 (L146P). In embodiments, the fibroblast growth factor is capable of activating FGFR1c, optionally wherein the activating requires β-Klotho as a coreceptor. In embodiments, the fibroblast growth factor comprises an amino acid sequence that is at least about 90%, or at least about 95%, or at least about 97%, or at least about 98%, or at least about 99% identical to an amino acid sequence selected from SEQ ID NOs: 66 to 68, 76 and 77.

In embodiments, the linker comprises a protease-cleavable polypeptide linker. In embodiments, the protease-cleavable polypeptide linker cleavable by a protease that is endogenous to a mammalian liver. In embodiments, the protease-cleavable linker is cleavable by a protease selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the protease-cleavable linker comprises a consensus recognition and/or cleavage site of a protease selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

In embodiments, the chimeric protein comprises two protease-cleavable polypeptide linkers. In embodiments, wherein the first protease-cleavable polypeptide linker is C terminal to the first domain and the second protease-cleavable polypeptide linker is N terminal to the second domain. In embodiments, the two protease-cleavable polypeptide linkers are cleavable by a protease that is endogenous to a mammalian liver. In embodiments, the two protease-cleavable polypeptide linkers are cleavable by a protease independently selected from caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers comprise consensus recognition and/or cleavage sites of a proteases independently selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the two protease-cleavable polypeptide linkers are cleavable by a protease independently comprises an amino acid sequence selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

In embodiments, the protease-cleavable polypeptide linkers are cleavable by a protease that is endogenous to a mammalian liver. In embodiments, the protease being selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin. In embodiments, the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74. In embodiments, the protease-cleavable polypeptide linker is C terminal to the first domain or N terminal to the second domain.

In embodiments, the first domain comprising a glucagon-like peptide-1 (GLP-1) receptor agonist and the protease-cleavable polypeptide linker is C terminal to the first domain; or the second domain comprises a glucagon-like peptide-1 (GLP-1) receptor agonist and the protease-cleavable polypeptide linker is N terminal to the second domain. In embodiments, the chimeric protein comprises two protease-cleavable polypeptide linkers, such protease-cleavable polypeptide linker independently selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74. In embodiments, the first protease-cleavable polypeptide linker is C terminal to the first domain and the second protease-cleavable polypeptide linker is N terminal to the second domain.

In embodiments, the linker comprises a hinge-CH2-CH3 Fc domain derived from IgG1. In embodiments, the IgG1 is human IgG1. In embodiments, the linker further comprises a hinge-CH2-CH3 Fc domain derived from IgG4. In embodiments, the IgG4 is human IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 79. In embodiments, the linker further comprises the linker comprises one or more joining linkers, such joining linkers independently selected from SEQ ID NOs: 4 to 50 or 79. In embodiments, the linker comprises two or more joining linkers each joining linker independently selected from SEQ ID NOs: 4 to 50 or 79; wherein one joining linker is N terminal to the hinge-CH2-CH3-Fc domain and another joining linker is C terminal to the hinge-CH2-CH3-Fc domain.

In embodiments, the polynucleotide is an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. In embodiments, the mmRNA further comprises a 5′-cap and/or a poly A tail.

In embodiments, the pharmaceutical composition further comprises a carrier. In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the polynucleotide is formulated as lipid nanoparticles (LNPs), a lipoplex, or a liposome.

In embodiments, the polynucleotide is formulated as lipid nanoparticles (LNPs). In embodiments, the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the polynucleotide (e.g., mmRNA of any of the embodiments disclosed herein).

In embodiments, the lipid nanoparticles comprise lipids selected from one or more of an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the polynucleotide (e.g., mmRNA of any of the embodiments disclosed herein). In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g., C12, a PEG-dimyristyloxypropyl (C14), a PEG-dipalmityloxypropyl (C16), or a PEG-distearyloxypropyl (C18)).

In embodiments, the lipid nanoparticles comprise an ionizable lipid, a PEG-lipid, a phospholipid and a structural lipid. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid. In embodiments, the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle, and/or wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the isolated polynucleotide administered by a parenteral administration. In embodiments, the parenteral administration is selected from intradermal, intramuscular, intraperitoneal, intraarticular, intravenous, subcutaneous, intraarterial or transdermal administration.

In embodiments, the chimeric protein is expressed in liver. In embodiments, the chimeric protein is cleaved in liver. In embodiments, the GLP-1 receptor agonist is released in liver. In embodiments, the GLP-1 receptor agonist enters circulation upon release in liver.

In embodiments, the subject is obese or at risk of obesity. In embodiments, the subject is suffering from metabolic syndrome. In embodiments, the subject has high blood glucose. In embodiments, the subject is high fed and fasting blood glucose.

In embodiments, an increase in the level of chimeric protein encoded by the isolated polynucleotide may be observed in tissue selected from the liver, spleen, kidney, lung, heart, peri-renal adipose tissue, thymus and muscle and/or in a bodily fluid selected from peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. The increased level of chimeric protein can be observed in the tissue and/or bodily fluid of the subject within 2, 8 and/or 24 hours after administration.

In aspects, the present disclosure provides a method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: N terminus—(a)-(b)-(c)—C terminus, wherein: A. (a) is a first domain comprising glucagon-like peptide-1 (GLP-1), (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor 19 (FGF19); or B. (a) is a first domain comprising a fibroblast growth factor 19 (FGF19), (b) is a linker adjoining the first domain and a second domain, optionally wherein the linker comprises one or more protease-cleavable polypeptide linkers, and/or a hinge-CH2-CH3 Fc domain, and (c) is the second domain comprising glucagon-like peptide-1 (GLP-1).

In aspects, the present disclosure provides a method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: N terminus—(a)-(b)-(c)—C terminus, wherein: A. (a) is a first domain comprising glucagon-like peptide-1 (GLP-1), (b) is a linker adjoining the first domain and a second domain, optionally wherein the linker comprises one or more protease-cleavable polypeptide linkers, and/or a hinge-CH2-CH3 Fc domain, and (c) is the second domain comprising a fibroblast growth factor 21 (FGF21); or B. (a) is a first domain comprising a fibroblast growth factor 21 (FGF21), (b) is a linker adjoining the first domain and a second domain, optionally wherein the linker comprises one or more protease-cleavable polypeptide linkers, and/or a hinge-CH2-CH3 Fc domain, and (c) is the second domain comprising glucagon-like peptide-1 (GLP-1).

In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

In embodiment, the modified nucleoside of methods and compositions of the present invention is m5° C. (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is Y (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl) adenosine); ms²i⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵ Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl) uridine); mchm⁵U (5-(carboxyhydroxymethyl) uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴ Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); Tm⁵U (5-taurinomethyluridine); Tm⁵s²U (5-taurinomethyl-2-thiouridine)); imG-14 (4-demethylwyosine); imG² (isowyosine); or ac⁶A (N⁶-acetyladenosine).

In embodiments, the mmRNA further comprises a 5′-cap and/or a poly A tail.

In embodiments, the pharmaceutical composition further comprises a carrier. In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the polynucleotide is formulated as lipid nanoparticles (LNPs), a lipoplex, or a liposome. In embodiments, the polynucleotide is formulated as lipid nanoparticles (LNPs). In embodiments, the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18); 1,2-dioleoyl-3-trimethylammoniumpropane (DOTAP); dioleoylphosphatidylethanolamine (DOPE); and the polynucleotide (e.g., mmRNA of any of the embodiments disclosed herein).

In embodiments, the lipid nanoparticles comprise lipids selected from one or more of an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the polynucleotide (e.g., mmRNA of any of the embodiments disclosed herein). In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g., C12, a PEG-dimyristyloxypropyl (C14), a PEG-dipalmityloxypropyl (C16), or a PEG-distearyloxypropyl (C18)).

In embodiments, the lipid nanoparticles comprise an ionizable lipid, a PEG-lipid, a phospholipid and a structural lipid. In embodiments, the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the isolated polynucleotide administered by a parenteral administration. In embodiments, the parenteral administration is selected from intradermal, intramuscular, intraperitoneal, intraarticular, intravenous, subcutaneous, intraarterial or transdermal administration. In embodiments, the chimeric protein is expressed in liver. In embodiments, the chimeric protein is cleaved in liver. In embodiments, the GLP-1 receptor agonist is released in liver. In embodiments, the GLP-1 receptor agonist enters circulation upon release in liver.

In embodiments, the treatment of NAFLD comprises a reduction in hepatic steatosis. In embodiments, the treatment of NAFLD comprises a reduction in hepatic inflammation. In embodiments, the NAFLD activity score (NAS) following administration is 7 or less. In embodiments, the NAS following administration is 5 or less. In embodiments, the NAS following administration is 3 or less. In embodiments, the subject has hepatic cirrhosis associated with the NAFLD. In embodiments, the subject has hepatic cirrhosis as a comorbidity. In embodiments, the subject has hepatic cirrhosis caused by the NAFLD. In embodiments, the NAFLD is NAFL with attendant liver cirrhosis.

In embodiments, the treatment of the NAFLD comprises treatment of liver cirrhosis. In embodiments, the treatment of NAFLD decreases the level of serum bile acids in the subject. In embodiments, the treatment of NAFLD comprises treatment of pruritus. In embodiments, the NAFLD is simple nonalcoholic fatty liver (NAFL). In embodiments, the treatment of NAFL comprises treatment of pruritus. In embodiments, the treatment of NAFL decreases the level of serum bile acids in the subject.

In embodiments, the NAFLD is nonalcoholic steatohepatitis (NASH). In embodiments, the NAFLD is NASH with attendant liver cirrhosis. In embodiments, the treatment of NASH decreases the level of serum bile acids in the subject. In embodiments, the treatment of NASH comprises treatment of pruritus.

In embodiments, an increase in the level of chimeric protein encoded by the isolated polynucleotide may be observed in tissue selected from the liver, spleen, kidney, lung, heart, peri-renal adipose tissue, thymus and muscle and/or in a bodily fluid selected from peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. The increased level of chimeric protein can be observed in the tissue and/or bodily fluid of the subject within 2, 8 and/or 24 hours after administration.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g. GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the disclosure pertains to veterinary use. In a specific embodiment, the non-human animal is a household pet. In another specific embodiment, the non-human animal is a livestock animal.

### Kits

The disclosure provides kits that can simplify the administration of any agent described herein. An illustrative kit of the disclosure comprises any composition described herein in unit dosage form. In embodiments, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In embodiments, the kit comprises a container containing an effective amount of a composition of the disclosure and an effective amount of another composition, such those described herein.

Any aspect or embodiment described herein can be combined with any other aspect or embodiment as disclosed herein.

The disclosure will be further described in the following examples, which do not limit the scope of the disclosure described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present disclosure, as exemplified by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Construction and Characterization of an Illustrative GLP-1- and FGF19-Based Chimeric Protein

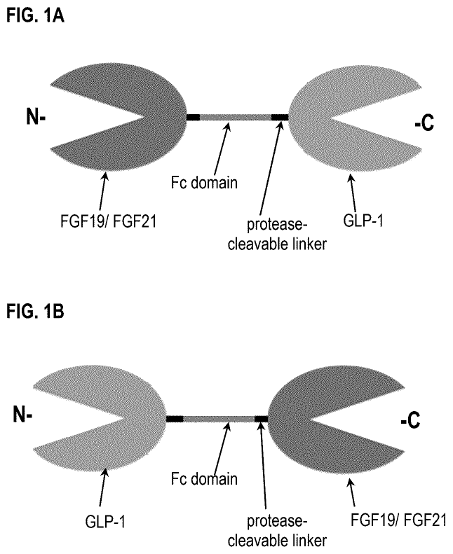

A construct encoding a GLP-1- and FGF19-based chimeric protein was generated. The “GLP-1-Fc-FGF19” construct included GLP-1 fused to FGF19 via a hinge-CH2-CH3 Fc domain derived from IgG1 and a protease-cleavable polypeptide linker. See, FIG. 1A.

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The GLP-1-Fc-FGF19 construct was transiently expressed in 293 cells and purified using protein-A affinity chromatography. To understand the native structure of the GLP-1-Fc-FGF19 chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the GLP-1-Fc-FGF19 chimeric protein, the gels were run in triplicates and probed using an anti-GLP-1 antibody, an anti-IgG+IgM (H+L) antibody, or an anti-FGF19 antibody.

### Example 2. Construction and Characterization of an Illustrative GLP-1- and FGF21-Based Chimeric Protein

A construct encoding a GLP-1- and FGF21-based chimeric protein was generated. The “GLP-1-Fc-FGF21” construct included GLP-1 fused to FGF21 via a hinge-CH2-CH3 Fc domain derived from IgG1 and a protease-cleavable polypeptide linker. See, FIG. 1B.

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with affinity chromatography binding resin columns.

The GLP-1-Fc-FGF21 construct was transiently expressed in CHO cells and purified using affinity chromatography. To understand the native structure of the GLP-1-Fc-FGF21 chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the GLP-1-Fc-FGF21 chimeric protein, the gels were run in triplicates and probed using an anti-GLP-1 antibody, an anti-IgG+IgM (H+L) antibody, or an anti-FGF21 antibody.

### Example 3. Further Characterization of the GLP-1- and FGF19/FGF21-Based Chimeric Proteins Disclosed Herein

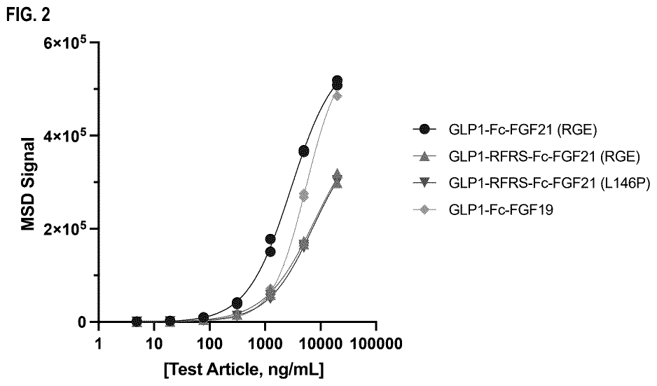

The binding of the chimeric proteins disclosed above to an anti-mouse FGF19 antibody or anti-mouse FGF21 antibody was measured using a Meso Scale Discovery (MSD) platform-based ELISA assay. Briefly, the anti-mouse FGF19 antibody or anti-mouse FGF21 antibody was coated on a plate. Increasing amounts of the mouse surrogates of human GLP-1-Fc-FGF21 (RGE), GLP-1-RFRS-Fc-FGF21 (RGE), GLP-1-RFRS-Fc-FGF21 (L146P), GLP-1-RFRS-Fc-FGF19 chimeric proteins were added to the plate for capture by the plate-bound anti-mouse FGF19 antibody or anti-mouse FGF21 antibody. The proteins captured by the plate-bound anti-mouse FGF19 antibody or anti-mouse FGF21 antibody were detected using an anti-mouse Fc antibody and a SULFO-TAG conjugated secondary antibody. As shown in FIG. 2, each of the human GLP-1-Fc-FGF21 (RGE), GLP-1-RFRS-Fc-FGF21 (RGE), GLP-1-RFRS-Fc-FGF21 (L146P), GLP-1-RFRS-Fc-FGF19 chimeric proteins bound to the FGF19 or FGF21 antibodies in a dose-dependent and saturable manner.

These results demonstrate, inter alia, that the chimeric proteins disclosed herein contain the above FGF19 or FGF21 variants and an Fc domain.

### Example 4. The Purified Chimeric Proteins and the Culture Supernatants of Cells Transfected with the Modified mRNA (mmRNA) Disclosed Herein Bind to GLP-1 Receptor

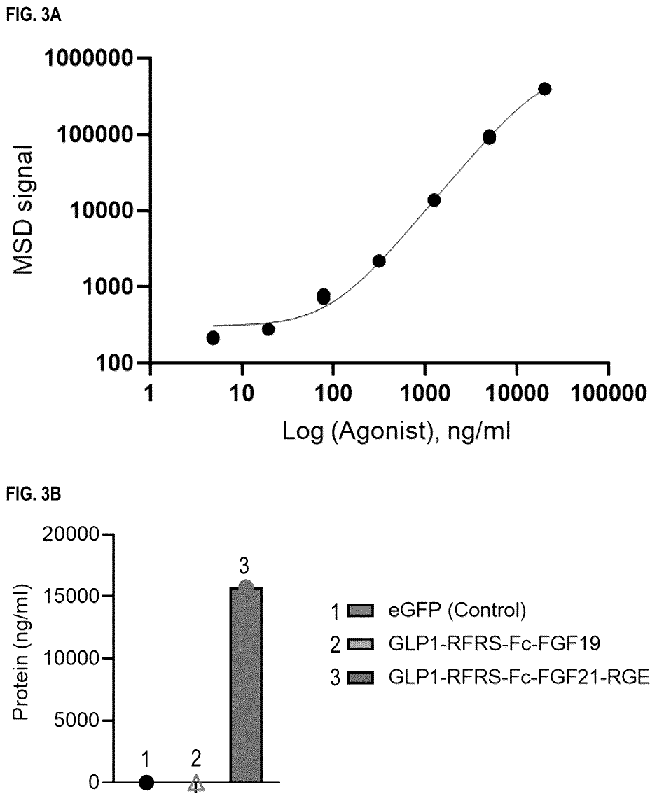

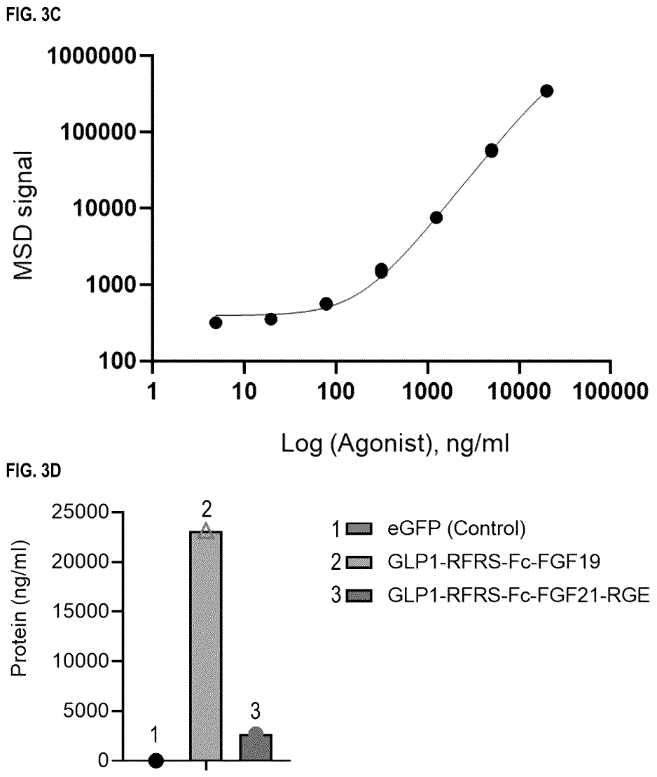

The binding of the purified GLP-1-RFRS-Fc-FGF21 (RGE) chimeric protein to human GLP-1 receptor was measured using a Meso Scale Discovery (MSD) platform-based ELISA assay. Briefly, recombinant human GLP-1 receptor was coated on a plate. Increasing amounts of the purified human GLP-1-RFRS-Fc-FGF21 (RGE) chimeric protein was added to the plate for capture by the plate-bound recombinant human GLP-1 receptor and detected using an anti-FGF21 antibody and a SULFO-TAG conjugated secondary antibody. As shown in FIG. 3A, the purified GLP-1-RFRS-Fc-FGF21 (RGE) chimeric protein showed a dose-dependent signal. These results demonstrate, inter alia, that the purified GLP-1-RFRS-Fc-FGF21 (RGE) chimeric protein binds to the human GLP-1 receptor.

The GLP-1 receptor binding by the human GLP-1-RFRS-Fc-FGF21 (RGE) chimeric protein secreted by cells transfected with a modified mRNA (mmRNA) encoding the GLP-1-RFRS-Fc-FGF21 (RGE) chimeric protein was assessed. Briefly, CHO-K1 cells were transfected with modified mRNA (mmRNA) encoding the GLP-1-RFRS-Fc-FGF19 or GLP-1-RFRS-Fc-FGF21 (RGE) chimeric proteins. Cells transfected with mmRNA encoding eGFP was used as a positive control. Culture supernatant was collected from 72 hours after transfection. The binding of the protein in the culture supernatant to human GLP-1 receptor was measured using a Meso Scale Discovery (MSD) platform-based ELISA assay. Briefly, recombinant human GLP-1 receptor was coated on a plate. The culture supernatant was added to the plate for capture by the plate-bound recombinant human GLP-1 receptor and detected using an anti-FGF21 antibody and a SULFO-TAG conjugated secondary antibody. The GLP-1-RFRS-Fc-FGF21 (RGE) chimeric protein in the culture supernatant was quantitated using the purified chimeric protein as a reference standard. As shown in FIG. 3B, the culture supernatant of the cells transfected with modified mRNA (mmRNA) encoding the GLP-1-RFRS-Fc-FGF21 (RGE) chimeric protein showed a signal equivalent to about 15,000 ng/ml of the purified protein. In contrast, supernatants the cells transfected with mmRNA encoding the GLP-1-RFRS-Fc-FGF19 chimeric protein or eGFP showed only a background level of signal (FIG. 3B). Since signal generation in this assay requires binding of both GLP-1 end of the molecule to GLP-1 receptor and the anti-FGF21 antibody to FGF21 (RGE), These results demonstrate, inter alia, that cells transfected with mmRNA encoding the GLP-1-RFRS-Fc-FGF21 (RGE) chimeric protein produce the GLP-1-RFRS-Fc-FGF21 (RGE) chimeric protein.

The binding of the purified GLP-1-RFRS-Fc-FGF19 chimeric protein to human GLP-1 receptor was measured using a Meso Scale Discovery (MSD) platform-based ELISA assay. Briefly, recombinant human GLP-1 receptor was coated on a plate. Increasing amounts of the purified human GLP-1-RFRS-Fc-FGF19 chimeric protein was added to the plate for capture by the plate-bound recombinant human GLP-1 receptor and detected using an anti-FGF19 antibody and a SULFO-TAG conjugated secondary antibody. As shown in FIG. 3C, the purified GLP-1-RFRS-Fc-FGF19 chimeric protein showed a dose-dependent signal. These results demonstrate, inter alia, that the purified GLP-1-RFRS-Fc-FGF19 chimeric protein binds to the human GLP-1 receptor.

The GLP-1 receptor binding by the human GLP-1-RFRS-Fc-FGF19 chimeric protein secreted by cells transfected with a modified mRNA (mmRNA) encoding the GLP-1-RFRS-Fc-FGF19 chimeric protein was assessed. Briefly, CHO-K1 cells were transfected with modified mRNA (mmRNA) encoding the GLP-1-RFRS-Fc-FGF19 or GLP-1-RFRS-Fc-FGF21 (RGE) chimeric proteins. Cells transfected with mmRNA encoding eGFP was used as a positive control. Culture supernatant was collected from 72 hours after transfection. The binding of the protein in the culture supernatant to human GLP-1 receptor was measured using a Meso Scale Discovery (MSD) platform-based ELISA assay. Briefly, recombinant human GLP-1 receptor was coated on a plate. The culture supernatant was added to the plate for capture by the plate-bound recombinant human GLP-1 receptor and detected using an anti-FGF19 antibody and a SULFO-TAG conjugated secondary antibody. The GLP-1-RFRS-Fc-FGF19 chimeric protein in the culture supernatant was quantitated using the purified chimeric protein as a reference standard. As shown in FIG. 3D, the culture supernatant of the cells transfected with modified mRNA (mmRNA) encoding the GLP-1-RFRS-Fc-FGF19 chimeric protein showed a signal equivalent to more than 20,000 ng/ml of the purified protein. In contrast, supernatants the cells transfected with mmRNA encoding the GLP-1-RFRS-Fc-FGF21 (RGE) chimeric protein or eGFP showed about 2000 ng and only a background level of signal, respectively (FIG. 3D). Since signal generation in this assay requires binding of both GLP-1 end of the molecule to GLP-1 receptor and the anti-FGF19 antibody to FGF19, these results demonstrate, inter alia, that cells transfected with mmRNA encoding the GLP-1-RFRS-Fc-FGF19 chimeric protein produce the GLP-1-RFRS-Fc-FGF19 chimeric protein.

Separately, binding affinity of GLP-1-Fc-FGF19 and GLP-1-Fc-FGF21 (RGE) to the GLP-1 receptor were also determined using bio-layer interferometry. The resulting Kd values were 13.2 pM for GLP-1-Fc-FGF19 and and 37.8 pM GLP-1-Fc-FGF21 (RGE).

### Example 5. The Purified Chimeric Proteins Disclosed Herein Bind to Cells Expressing GLP-1 Receptor and FGF Receptors

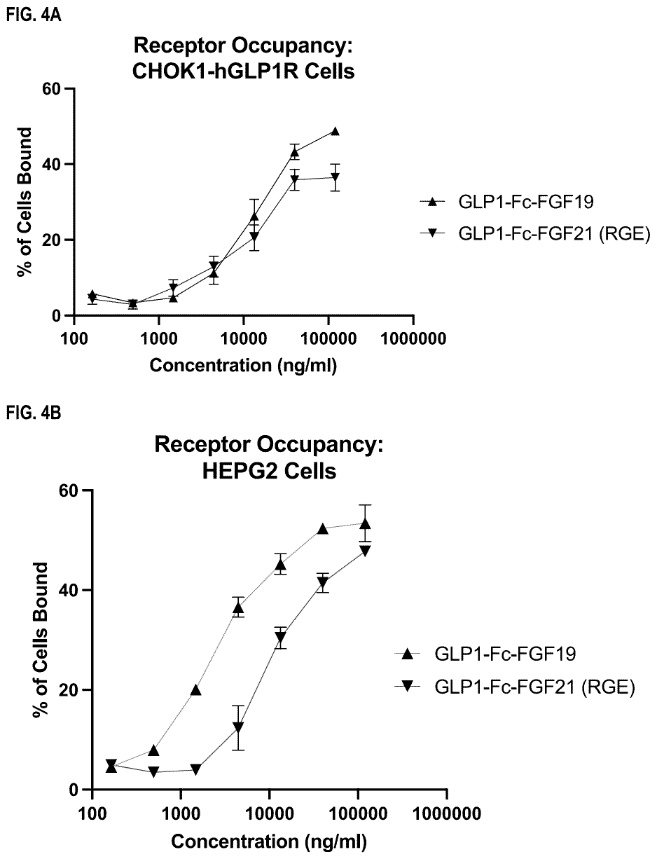

To understand whether the GLP-1 part of the GLP-1-RFRS-Fc-FGF21 (RGE) and GLP-1-RFRS-Fc-FGF19 chimeric proteins can specifically bind cells expressing GLP-1 receptor, CHO-K1 cells expressing GLP-1 receptor were generated using standard techniques. A confirmed GLP-1 receptor-expressing CHO-K1 clone was used for further studies. To determine whether the GLP-1-RFRS-Fc-FGF21 (RGE) and GLP-1-RFRS-Fc-FGF19 chimeric proteins can specifically bind the GLP-1 receptor-expressing CHO-K1 cells, a flow-cytometry-based assay was carried out. The GLP-1 receptor-expressing CHO-K1 cells were incubated with increasing concentrations of the GLP-1-RFRS-Fc-FGF21 (RGE) and GLP-1-RFRS-Fc-FGF19 chimeric proteins, and stained with a secondary agent specific to the chimeric proteins, and analyzed by flow cytometry. As shown in FIG. 4A, the GLP-1-RFRS-Fc-FGF21 (RGE) and GLP-1-RFRS-Fc-FGF19 chimeric proteins bound the GLP-1 receptor-expressing CHO-K1 cells in a dose-dependent and saturable manner. These results demonstrate, inter alia, that the GLP-1 end of the GLP-1-RFRS-Fc-FGF21 (RGE) and GLP-1-RFRS-Fc-FGF19 chimeric proteins specifically binds to cells expressing GLP-1 receptor.

To understand whether the FGF part of the GLP-1-RFRS-Fc-FGF21 (RGE) and GLP-1-RFRS-Fc-FGF19 chimeric proteins can specifically bind cells expressing FGF receptors, the hepatocyte-like human hepatoblastoma HepG2 cells, which express FGF receptors such as FGFR1, FGFR2, FGFR3 and FGFR4 were used. Kan et al., Specificity for Fibroblast Growth Factors Determined by Heparan Sulfate in a Binary Complex with the Receptor Kinase, Journal of Biological Chemistry 274(22): 15947-15952 (1999). To determine whether the GLP-1-RFRS-Fc-FGF21 (RGE) and GLP-1-RFRS-Fc-FGF19 chimeric proteins can specifically bind the HepG2 cells, a flow-cytometry-based assay was carried out. The HepG2 cells were incubated with increasing concentrations of the GLP-1-RFRS-Fc-FGF21 (RGE) and GLP-1-RFRS-Fc-FGF19 chimeric proteins, stained with a secondary agent specific to the chimeric proteins, and analyzed by flow cytometry. As shown in FIG. 4B, the GLP-1-RFRS-Fc-FGF21 (RGE) and GLP-1-RFRS-Fc-FGF19 chimeric proteins bound HepG2 cells in a dose-dependent and saturable manner. These results demonstrate, inter alia, that the FGF21 (RGE) or FGF19 ends of the GLP-1-RFRS-Fc-FGF21 (RGE) and GLP-1-RFRS-Fc-FGF19 chimeric proteins specifically binds to cells expressing FGF receptors.

### Example 6. Modified mRNA (mmRNA) Disclosed Herein Drives the Production of the Active GLP-1- and FGF19/FGF21-Based Chimeric Proteins

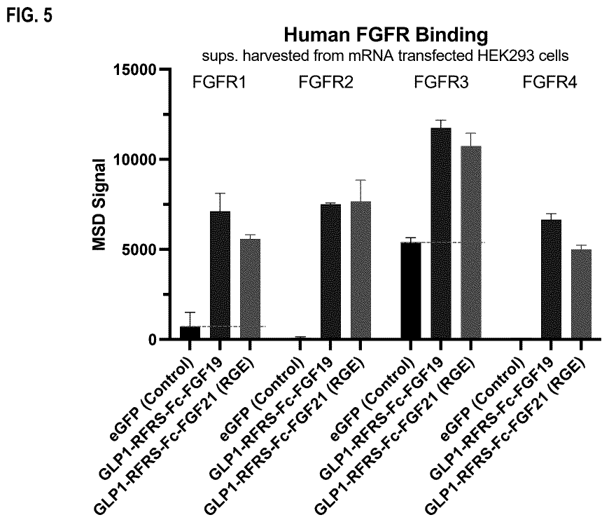

HEK293 cells were transfected with the modified mRNA (mmRNA) encoding the GLP-1-RFRS-Fc-FGF19 or GLP-1-RFRS-Fc-FGF21 (RGE) chimeric proteins disclosed herein and the transfected HEK293 cells were cultured. The GLP-1-RFRS-Fc-FGF19 or GLP-1-RFRS-Fc-FGF21 (RGE) chimeric proteins secreted by the transfected HEK293 cells were assayed for contemporaneous binding to recombinant FGF receptors FGFR1, FGFR2, FGFR3 or FGFR4 and recombinant GLP-1 receptor (GLP-1R) using a Meso Scale Discovery (MSD) platform-based ELISA assay. Briefly, the recombinant FGF receptors FGFR1, FGFR2, FGFR3 or FGFR4 were coated on a plate. Culture supernatants of HEK293 cells transfected with mmRNA encoding human GLP-1-RFRS-Fc-FGF19 or GLP-1-RFRS-Fc-FGF21 (RGE) chimeric proteins or the eGFP protein were added to the plate for capture by the plate-bound recombinant FGF receptors. The eGFP protein was used as a negative control. The proteins captured by the plate-bound recombinant FGF receptors were detected using recombinant GLP-1 receptor (GLP-1R), an anti-GLP-1R antibody and a SULFO-TAG conjugated secondary antibody. In this case, generation of signal requires contemporaneous binding to both the recombinant FGF receptor and GLP-1R. As shown in FIG. 5, the culture supernatants of HEK293 cells transfected with mmRNA encoding human GLP-1-RFRS-Fc-FGF19 or GLP-1-RFRS-Fc-FGF21 (RGE) chimeric proteins produced a signal corresponding to binding to each of FGF receptors FGFR1, FGFR2, FGFR3 or FGFR4 and GLP-1R. In contrast, the culture supernatants of HEK293 cells expressing eGFP generated only a background level of signal.

These results demonstrate, inter alia, that (1) the modified mRNA (mmRNA) disclosed herein encoding the chimeric proteins of the present disclosure drive the production of functional chimeric protein capable of binding their respective receptors, and (2) chimeric proteins of the present disclosure are capable of contemporaneously binding to the receptors of FGF and GLP-1 proteins.

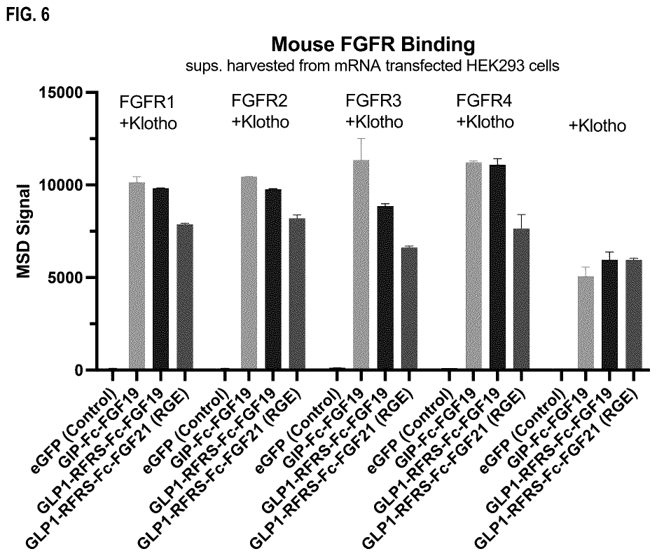

HEK293 cells were transfected with the modified mRNA (mmRNA) encoding the GLP-1-Fc-FGF19, GLP-1-RFRS-Fc-FGF19, or GLP-1-RFRS-Fc-FGF21 (RGE) chimeric proteins and the transfected HEK293 cells were cultured. The GLP-1-Fc-FGF19, GLP-1-RFRS-Fc-FGF19, and GLP-1-RFRS-Fc-FGF21 (RGE) chimeric proteins secreted by the transfected HEK293 cells were assayed for contemporaneous binding to (1) Klotho alone or in combination with a recombinant mouse FGF receptor FGFR1, FGFR2, FGFR3 or FGFR4, and (2) recombinant GLP-1 receptor (GLP-1R) using a Meso Scale Discovery (MSD) platform-based ELISA assay. Briefly, the Klotho alone or in combination with one of mouse FGFR1, FGFR2, FGFR3 or FGFR4 was coated on a plate. Culture supernatants of HEK293 cells transfected with mmRNA encoding human GLP-1-Fc-FGF19, GLP-1-RFRS-Fc-FGF19, and GLP-1-RFRS-Fc-FGF21 (RGE) chimeric proteins or the eGFP protein were added to the plate for capture by the plate-bound recombinant FGF receptors. The eGFP protein was used as a negative control. The proteins captured by the plate-bound recombinant FGF receptors were detected using recombinant mouse GLP-1 receptor (GLP-1R), an anti-GLP-1R antibody and a SULFO-TAG conjugated secondary antibody. In this case, generation of signal requires contemporaneous binding to both the recombinant FGF receptor and GLP-1R. As shown in FIG. 6, the culture supernatants of HEK293 cells transfected with mmRNA encoding human GLP-1-Fc-FGF19, GLP-1-RFRS-Fc-FGF19, and GLP-1-RFRS-Fc-FGF21 (RGE) chimeric proteins produced a signal corresponding to Klotho alone. When the combination of Klotho and mouse FGFR1, FGFR2, FGFR3 or FGFR4 was coated on the plate, a higher amount of MSD signal was detected compared to plates having of Klotho alone coated (FIG. 6). As expected, the culture supernatants of HEK293 cells expressing eGFP generated only a background level of signal (FIG. 6).

These results demonstrate, inter alia, (1) the expression of functional chimeric protein driven in cells by the modified mRNA (mmRNA) disclosed herein, (2) contemporaneous binding of the chimeric proteins disclosed herein to the receptors of FGF and GLP-1 proteins, (3) the cross-reactivity of the human FGF proteins present in the chimeric proteins disclosed herein with mouse FGF receptors, and (4) binding of FGF proteins present in the chimeric proteins disclosed herein both to Klotho and FGF receptors, and increased binding to the combination of Klotho/FGFR1, Klotho/FGFR2, Klotho/FGFR3 or Klotho/FGFR4 compared to the binding to Klotho alone.

### Example 7. The Purified Chimeric Proteins and the Culture Supernatants of Cells Transfected with the Modified mRNA (mmRNA) Disclosed Herein Activate GLP-1 Receptor

The activity of GLP-1 receptor was assayed using the GeneBLAzer® GLP-1R-CRE-bla CHO-K1 cell-based assay (ThermoFisher) and purified proteins. This assay is diagrammatically represented in FIG. 7A. Upon binding of a glucagon-like peptide-1 (GLP-1) receptor agonist to the targeting construct, a transcriptional cascade is initiated that produces β-lactamase (FIG. 7A). In the presence of the β-lactamase LiveBLAzer™ substrate, cells expressing β-lactamase fluoresce blue (460 nm), while those not expressing β-lactamase fluoresce green (530 nm); higher 460/530 ratio indicates the activation of GLP-1R.

Briefly, GeneBLAzer® GLP-1R-CRE-bla CHO-K1 cells were added to wells of black-wall assay plates. Medium alone (negative control) or increasing amounts of the GLP-1-His, GLP-1-RFRS-His proteins, or the purified GLP-1-Fc-FGF19, and GLP-1-RFRS-Fc-FGF21 (RGE) chimeric proteins were added to the plates and the plates were incubated in a humidified 37° C./5% CO₂ incubator for 5 hours. GLP-1-His, GLP-1-RFRS-His proteins were used as a positive control. To each well, LiverBLAzer-FRET B/G substrate mixture was added. The plate was covered with plate sealer and incubated at room temperature for 2 hours in the dark. Fluorescence emission was measured on SpectraMax plate reader at 460 nm and 530 nm with excitation set to 409 nm. After subtraction of background, average value at 460 nm (blue color) was divided by average value at 530 nm (green color) to obtain the 460/530 ratio. As shown in FIG. 7B, each of the GLP-1-His, GLP-1-RFRS-His proteins and the purified GLP-1-Fc-FGF19, and GLP-1-RFRS-Fc-FGF21 (RGE) chimeric proteins produced a dose-dependent and saturable increase in 460/530 ratio, indicative of the activation of GLP-1R. The recombinant purified GLP-1-His, GLP-1-RFRS-His proteins activated GLP-1R with an EC₅₀ of 192.5 and 35.9 nM, respectively (FIG. 7B). Surprisingly, the purified GLP-1-Fc-FGF19, and GLP-1-RFRS-Fc-FGF21 (RGE) chimeric proteins activated GLP-1R with an EC₅₀ of 0.65 and 0.44 nM, respectively, which is 2 orders of magnitude lower compared to the recombinant purified GLP-1-His, GLP-1-RFRS-His proteins (FIG. 7B).

These results indicate, inter alia, that chimeric proteins of the present disclosure activate the GLP-1R receptor with a higher potency compared to the recombinant purified GLP-1-His, GLP-1-RFRS-His proteins.

The activity of GLP-1 receptor was assayed using the GeneBLAzer® GLP-1R-CRE-bla CHO-K1 cell-based assay (ThermoFisher) and culture supernatants of HEK293 cells transfected with mmRNA encoding eGFP protein, human GLP-1-RFRS-Fc-FGF19, and GLP-1-RFRS-Fc-FGF21 (RGE) chimeric proteins or X-Fc-FGF19 chimeric protein that lacks GLP-1 and includes an unrelated peptide hormone X.

Briefly, GeneBLAzer® GLP-1R-CRE-bla CHO-K1 cells were added to wells of black-wall assay plates. Medium alone (negative control) or undiluted or 1:2, 1:4 or 1:8 dilutions of the culture supernatants of HEK293 cells transfected with mmRNA encoding eGFP protein, human GLP-1-RFRS-Fc-FGF19, and GLP-1-RFRS-Fc-FGF21 (RGE) chimeric proteins or X-Fc-FGF19 chimeric protein were added to the plates and the plates were incubated in a humidified 37° C./5% CO₂ incubator for 5 hours. The culture supernatants of HEK293 cells transfected with mmRNA encoding eGFP protein or the X-Fc-FGF19 chimeric protein that lacks GLP-1 and includes an unrelated peptide hormone X were used as negative controls. To each well, LiverBLAzer-FRET B/G substrate mixture was added. The plate was covered with plate sealer and incubated at room temperature for 2 hours in the dark. Fluorescence emission was measured on SpectraMax plate reader at 460 nm and 530 nm with excitation set to 409 nm. After subtraction of background, average value at 460 nm (blue color) was divided by average value at 530 nm (green color) to obtain the 460/530 ratio. As shown in FIG. 7C, each of the culture supernatants of HEK293 cells transfected with mmRNA encoding human GLP-1-RFRS-Fc-FGF19, and GLP-1-RFRS-Fc-FGF21 (RGE) chimeric proteins produced a dose-dependent increase in 460/530 ratio, indicative of the activation of GLP-1R (FIG. 7C). on the other hand, the culture supernatants of HEK293 cells transfected with mmRNA encoding eGFP protein or the X-Fc-FGF19 chimeric protein that lacks GLP-1 and includes an unrelated peptide hormone X produced only a background signal (FIG. 7C).

Without wishing to be bound by theory, the activation of GLP-1R leads to temporary depletion of ATP because of consumption of ATP in cyclization reaction to produce CAMP as well as by phosphorylation by protein kinase A of its substrate. Without wishing to be bound by theory, the temporary depletion of ATP leads to an inhibition of luciferase activity, which needs ATP as a substrate. The activation of GLP-1R receptor, induced by the modified mRNA (mmRNA) disclosed herein was assayed using a different experimental system.

Briefly, CHO-K1 cells expressing GLP-1 receptor were generated using standard techniques. A confirmed GLP-1 receptor-expressing CHO-K1 clone was used for further studies. The CHO-K1 cells expressing GLP-1 receptor were treated with increasing amounts of the culture supernatants of HEK293T cells, which were transfected with mmRNA encoding human GLP-1-Fc-FGF19, and GLP-1-Fc-FGF21 (RGE) chimeric proteins. Culture supernatants of un-transfected control HEK293T cells was used as a negative control. ATP levels in the cells, which are decreased as more CAMP is produced in response to the activation of GLP-1 receptor, were detected. As shown in FIG. 7D, each of the culture supernatants of HEK293T cells transfected with mmRNA encoding human GLP-1-Fc-FGF19, and GLP-1-Fc-FGF21 (RGE) chimeric proteins produced a dose-dependent, and saturable decrease in the levels of ATP, consistent with the activation of the GLP-1 receptor. On the other hand, the culture supernatants of un-transfected control HEK293T cells produced no change in the signal (FIG. 7D).

These results demonstrate, inter alia, that the chimeric proteins of the present disclosure and the protein secreted of by the cells transfected with the modified mRNA (mmRNA) encoding the chimeric proteins disclosed herein activate the GLP-1R receptor.

### Example 8. The Chimeric Proteins a Disclosed Herein Activate FGFR/Klotho Receptor

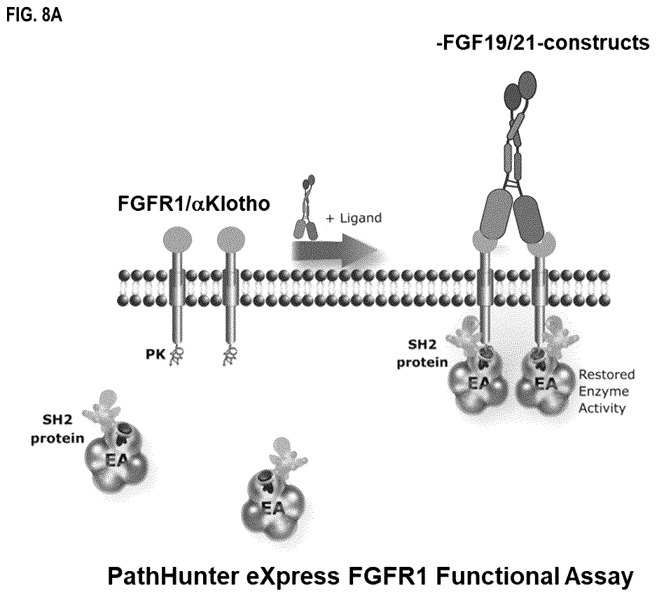

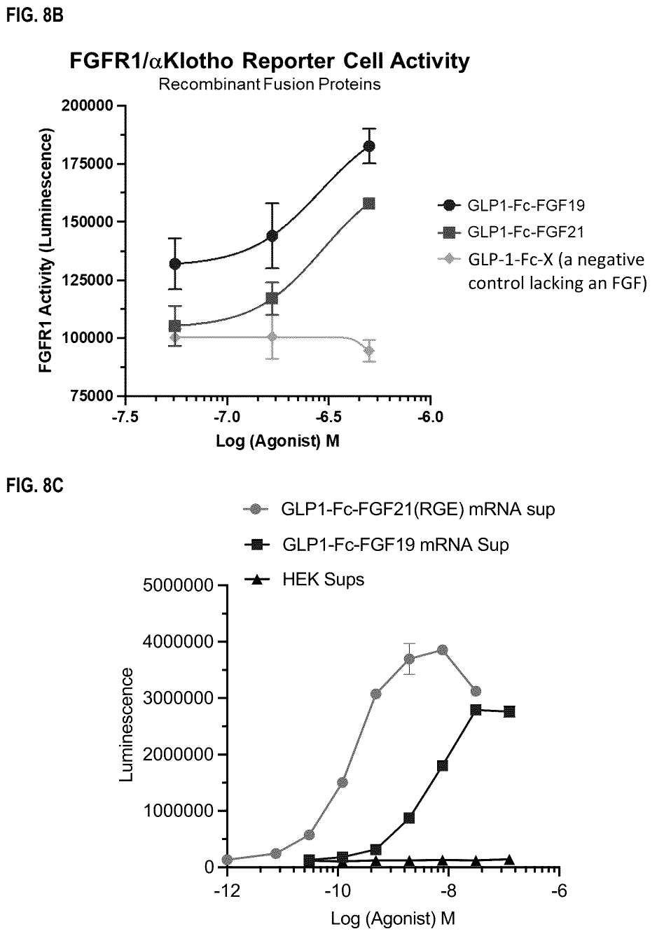

The activity of FGFR/Klotho receptor was assayed using the PathHunter express FGFR1 functional assay. This assay is diagrammatically represented in FIG. 8A. Upon binding of a chimeric protein, the FGFR1/Klotho receptor dimerizes, leading to the recruitment of a ProLink™ (PK) tagged RTK, an Enzyme Acceptor (EA) tagged SH2 domain, and forcing complementation of the two β-galactosidase enzyme fragments (EA and PK). The resulting functional enzyme hydrolyzes substrate to generate a chemiluminescent signal (FIG. 8A).

Briefly, PathHunter® RTK Functional cells were added to wells of black-wall assay plates and increasing amounts of the purified GLP-1-Fc-FGF19, and GLP-1-RFRS-Fc-FGF21 (RGE), and GLP-1-Fc-X chimeric proteins were added to the plates and the plates were incubated in a humidified 37° C./5% CO₂ incubator for 5 hours. The GLP-1-Fc-X chimeric protein that lacks FGF and includes an unrelated peptide hormone X was used as a negative control. To each well, a luminogenic β-galactosidase substrate mixture was added and luminescence was measured. As shown in FIG. 8B, each of the purified GLP-1-Fc-FGF19, and GLP-1-RFRS-Fc-FGF21 (RGE) chimeric proteins produced a dose-dependent increase in luminescence, indicative of the activation of FGFR1. On the other hand, the GLP-1-Fc-X chimeric protein, which lacks FGF and includes an unrelated peptide hormone X, produced only a background signal (FIG. 8B).

PathHunter® RTK Functional cells were added to wells of black-wall assay plates and increasing amounts of the culture supernatants of HEK293T cells, which were transfected with mmRNA encoding human GLP-1-Fc-FGF19, and GLP-1-Fc-FGF21 (RGE) chimeric proteins were added to the plates and the plates were incubated in a humidified 37° C./5% CO₂ incubator for 5 hours. The culture supernatants of un-transfected control HEK293T cells was used as a negative control. To each well, a luminogenic β-galactosidase substrate mixture was added and luminescence was measured. As shown in FIG. 8C, the culture supernatants of HEK293T cells transfected with mmRNA encoding human GLP-1-Fc-FGF19, and GLP-1-Fc-FGF21 (RGE) chimeric proteins produced a dose-dependent and saturable increase in luminescence, indicative of the activation of FGFR1. On the other hand, the culture supernatants of un-transfected control HEK293T cells produced only a background signal (FIG. 8C).

These results indicate, inter alia, that the chimeric proteins of the present disclosure and the protein secreted of by the cells transfected with the modified mRNA (mmRNA) encoding the chimeric proteins disclosed herein activate the FGFR1 receptor.

### Example 9. The Chimeric Proteins a Disclosed Herein Activate GLP-1 Receptor

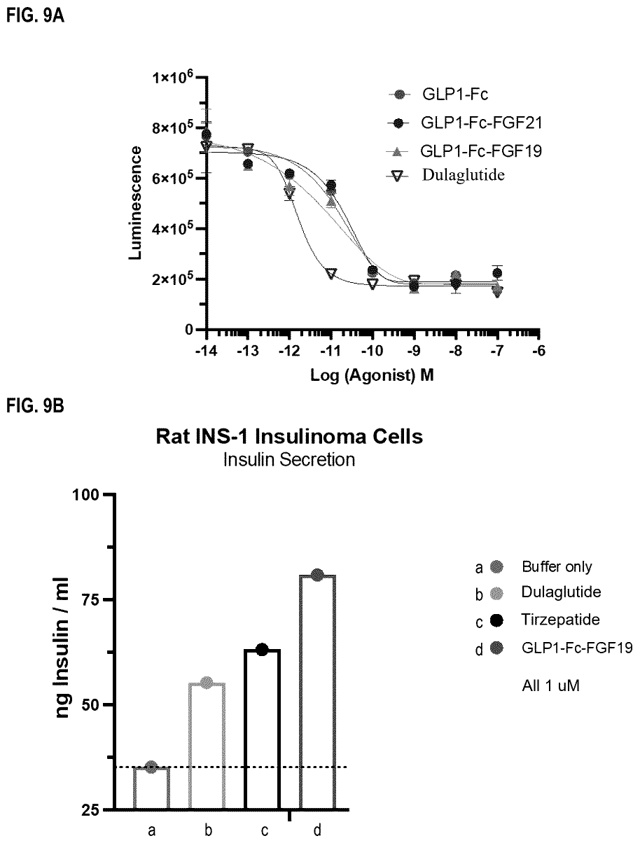

A derivative of the rat INS-1 insulinoma cell line, which is a commonly used model to study pancreatic islet beta cell function following glucose stimulation, was used to study the activation of GLP-1R and glucose-stimulated insulin secretion (GSIS) in vitro. Rat insulinoma INS-1 cells harboring a CAMP-luciferase reporter gene were used to study the activation of GLP-1R. Without wishing to be bound by theory, the activation of GLP-1R leads to temporary depletion of ATP because of consumption of ATP in cyclization reaction to produce cAMP as well as by phosphorylation by protein kinase A of its substrate. Without wishing to be bound by theory, the temporary depletion of ATP leads to an inhibition of luciferase activity, which needs ATP as a substrate. Briefly, the rat insulinoma INS-1 cells harboring a cAMP-luciferase reporter gene were stimulated for 1 hour with glucose, followed by a 2 hour incubation with increasing amounts of GLP-1R by the GLP-1-Fc fusion protein, the GLP-1-Fc-FGF19 or GLP-1-Fc-FGF21 chimeric proteins, or dulaglutide. Luciferase activity was measured using the CAMP-Glo™ assay (Promega). As shown in FIG. 9A, each of the GLP-1-Fc fusion protein, the GLP-1-Fc fusion protein, the GLP-1-Fc-FGF19 or GLP-1-Fc-FGF21 chimeric proteins, or dulaglutide caused a dose-dependent and saturable inhibition of luciferase.

These results indicate, inter alia, that the GLP-1-Fc-FGF19 or GLP-1-Fc-FGF21 chimeric proteins activate the GLP-1 receptor.

Rat insulinoma INS-1 cells harboring a cAMP-luciferase reporter gene were used to study in vitro glucose-stimulated insulin secretion (GSIS). Briefly, the rat insulinoma INS-1 cells harboring a CAMP-luciferase reporter gene were stimulated for 1 hour with glucose, followed by a 2 hour incubation with increasing amounts of dulaglutide, tirzepatide, or the GLP-1-Fc-FGF19 chimeric protein. Dulaglutide and tirzepatide were used as positive controls and buffer alone was used as a negative control. After 2 hours of incubation, insulin was quantitated in the culture supernatant using ELIZA. As shown in FIG. 9B, the GLP-1-Fc-FGF19 chimeric protein stimulated insulin production to a similar, if not greater, magnitude compared to dulaglutide or tirzepatide.

These results indicate, inter alia, that the chimeric proteins disclosed herein induce insulin production by pancreatic islet beta cells.

### Example 10. In Vivo Control of Non-Alcoholic Steatohepatitis (NASH) and Cirrhosis by the Purified Chimeric Proteins and Modified mRNA (mmRNA) Encoding the Chimeric Proteins Disclosed Herein

GLP-1/FGF21 fusion protein and mRNA constructs were assessed in commonly used models of obesity/type 2 diabetes shown in FIG. 10A. Briefly, male mice (C57BL6) were of approximately 13 weeks of age were housed 4 mice per cage. Following 2 weeks of acclimation, mice were introduced to 60% kcal HFD (Research Diets D12492). Cages were changed once, then changed once a week. Weekly body weights and non-fasted blood glucose were collected. Upon induction of obesity using high fat diet, the mice were randomly divided in the following 7 treatment groups:
Group #1 Untreated,
Group #2 dulaglutide (2.5 mpk/18 nmol/kg): dose weekly,
Group #3 Human GLP-1-Fc-FGF21 Fusion protein (5 mpk), and
Group #3 Human GLP-1-Fc-FGF21 mRNA (0.35 mpk)

Mice were injected by tail vein with vehicle and the drugs on Day 0, Day 4, Day 7, Day 10, Day 14, and Day 18. Body weights were measured every 2 days. Change in body weight was calculated and plotted. As shown in FIG. 10B, the body weights of control mice that received only PBS slightly increased during the course of the experiment (Group 1, closed circles in FIG. 10B). On the other hand, the treatment with each of dulaglutide, the GLP-1-Fc-FGF21 fusion protein, and the mmRNA encoding the GLP-1-Fc-FGF21 fusion protein induced a progressive weight loss (FIG. 10B).

Cumulative food intake was measured between days 7 and 10 and represented as average daily food intake. For food consumption, remaining food was weighed on day 11 and subtracted from the starting weight. This value was then normalized to the number of mice in the cage, and the number of days the mice had access to the food prior to weighing. As shown in FIG. 10C, the food intake of mice treated with each of dulaglutide, the GLP-1-Fc-FGF21 fusion protein, and the mmRNA encoding the GLP-1-Fc-FGF21 fusion protein decreased compared to the untreated mice (FIG. 10C).

Plasma insulin and triglyceride levels were measured. As shown in FIG. 10D (left panel), the plasma insulin levels of mice treated with each of dulaglutide, the GLP-1-Fc-FGF21 fusion protein, and the mmRNA encoding the GLP-1-Fc-FGF21 fusion protein decreased compared to the untreated mice. Plasma triglycerides levels were measured. As shown in FIG. 10D (right panel), the plasma triglycerides levels of mice treated with each of dulaglutide, the GLP-1-Fc-FGF21 fusion protein, and the mmRNA encoding the GLP-1-Fc-FGF21 fusion protein decreased compared to the untreated mice.

The levels of liver Lipin1 mRNA and Glut4 mRNA were measured. As shown in FIG. 10E (left panel), the liver Lipin1 mRNA levels of mice treated with each of dulaglutide, the GLP-1-Fc-FGF21 fusion protein, and the mmRNA encoding the GLP-1-Fc-FGF21 fusion protein decreased compared to the untreated mice. As shown in FIG. 10E (right panel), the liver Glut4 mRNA levels of mice treated with each of dulaglutide, the GLP-1-Fc-FGF21 fusion protein, and the mmRNA encoding the GLP-1-Fc-FGF21 fusion protein decreased compared to the untreated mice. Surprisingly, lipin1 was downregulated more in mice treated with the GLP-1-Fc-FGF21 fusion protein and the mmRNA encoding the GLP-1-Fc-FGF21 fusion protein compared to the mice treated with dulaglutide (FIG. 10E). Therefore, it is expected that the GLP-1-Fc-FGF21 fusion protein and the mmRNA encoding the GLP-1-Fc-FGF21 fusion protein is effective against hyperglycemia and insulin resistance. See Ryu et al., TORC2 Regulates Hepatic Insulin Signaling via a Mammalian Phosphatidic Acid Phosphatase, LIPIN1, Cell Metabolism 9:240-251 (2009).

These results indicate, inter alia, that the chimeric proteins disclosed herein and the mmRNA encoding the chimeric proteins disclosed herein control body weight, plasma insulin, food intake, hyperglycemia and insulin resistance. These results further indicate, inter alia, that the chimeric proteins disclosed herein and the mmRNA encoding the chimeric proteins disclosed herein are useful to treat diabetes, obesity, Type II diabetes, metabolic syndrome, hyperglycemia insulin resistance and related ailments.

### Example 12. In Vivo Control of Liver Fibrosis, Steatosis, Body Weight, Plasma Glucose, Food Intake, and Body Fat by the Purified Chimeric Proteins and Modified mRNA (mmRNA) Encoding the Chimeric Proteins Disclosed Herein

Various parameters related to fatty liver disease (steatosis) early-stage liver fibrosis model were assessed in a mouse model shown in FIG. 10A. Briefly, C57BL/6 mice were of approximately 13 weeks of age were housed 4 mice per cage. Following 2 weeks of acclimation, mice were introduced to 60% kcal HFD (Research Diets D12492). Cages were changed once, then changed once a week. Following their maintenance on the high fat diet for 24 weeks they were randomly assigned to the following treatment groups:
Group #1 Vehicle (PBS)
Group #2 Tirzepatide (0.5 mpk)
Group #3 Human GLP-Fc-FGF19 fusion protein (5 mpk)
Group #4 Human GLP-Fc-FGF19 mRNA/LNP (1 mpk)

The mice of groups 1, 3 and 4 were administered tail vein injections of the vehicle, purified fusion protein or mmRNA encoding the fusion protein on Day 0, Day 4, Day 7, Day 10, Day 14, and Day 18. Tirzepatide was administered on days 0, 7, and 14 due poor tolerability and toxicity in mice, causing diarrhea, dehydration, lethargic behavior, scruffy fur, and moribund appearance. Similar adverse reactions not observed with the other constructs. Body weights were measured every 2 days. Change in body weight was calculated and plotted.

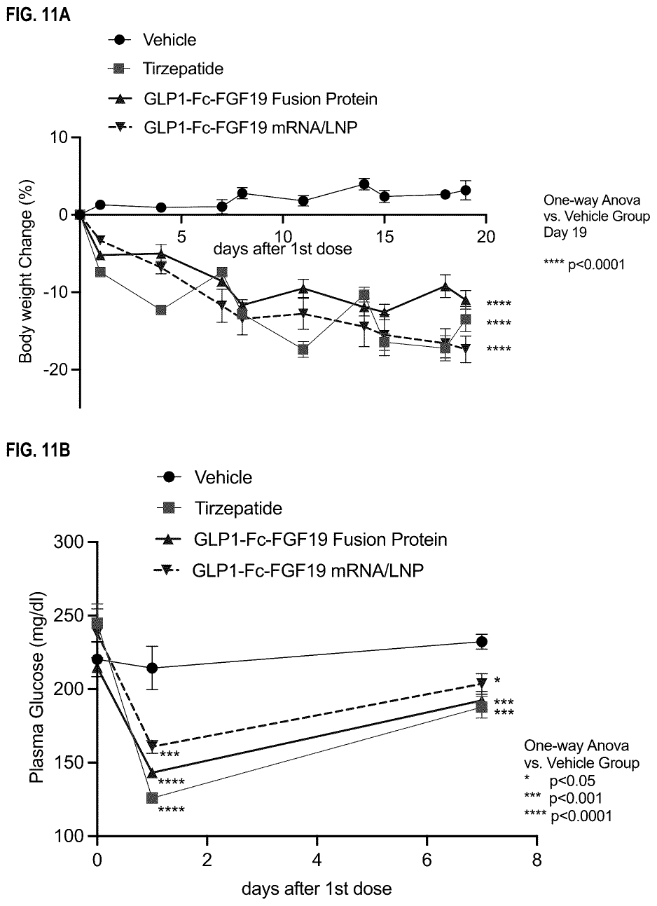

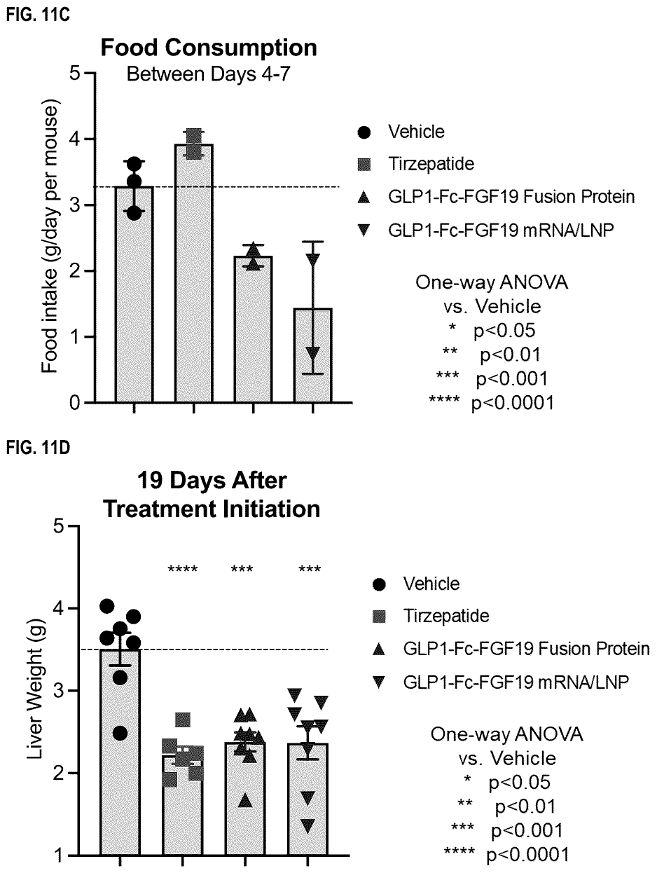

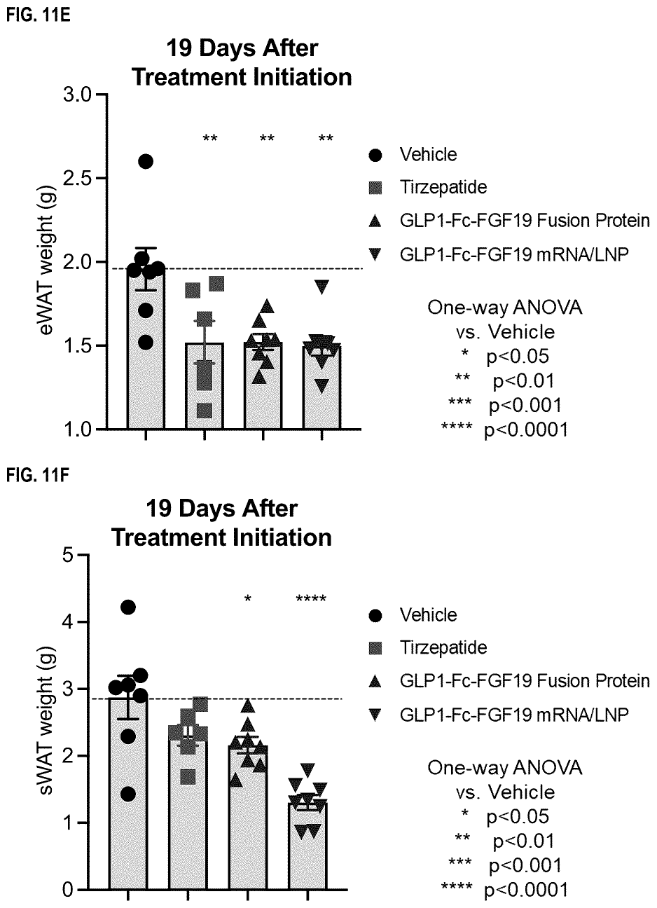

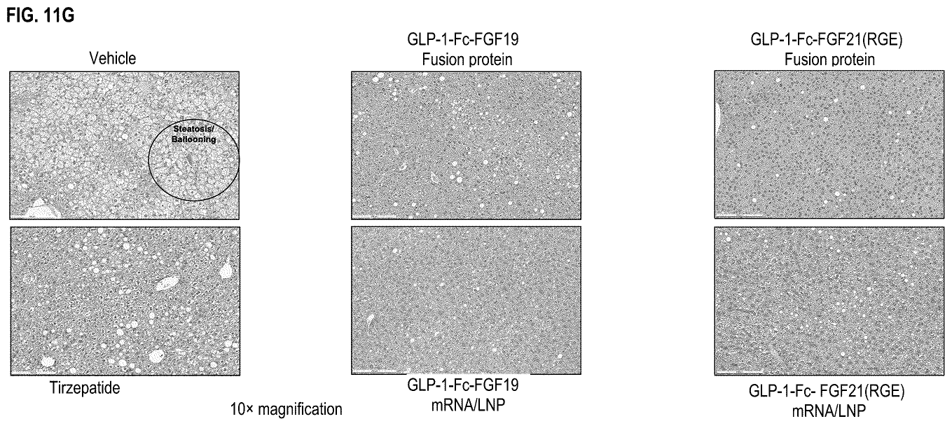

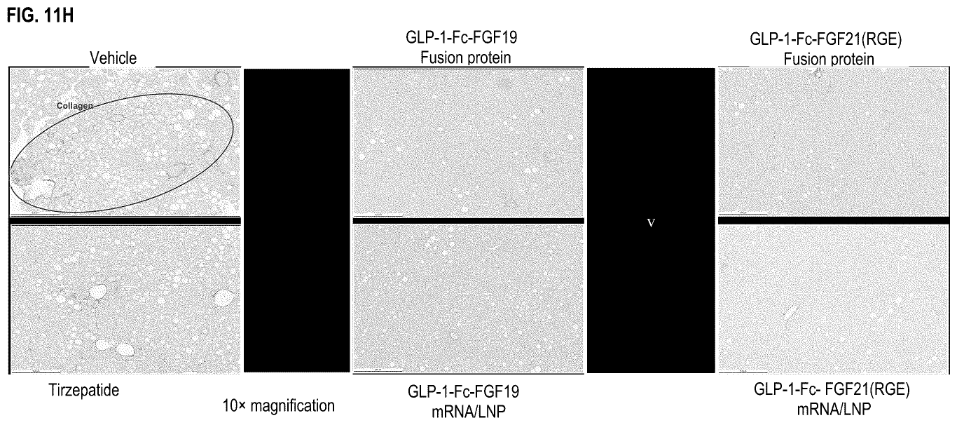

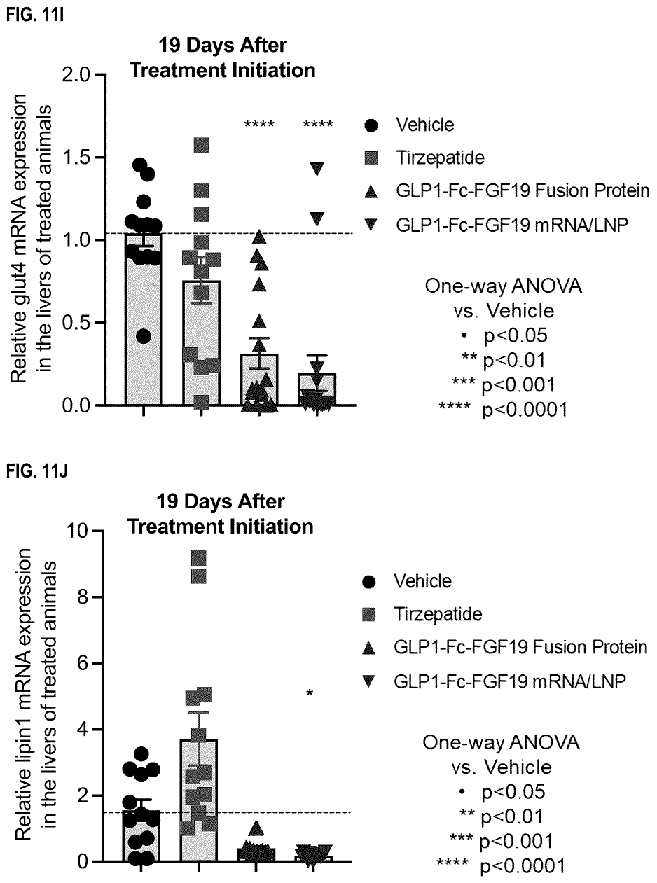

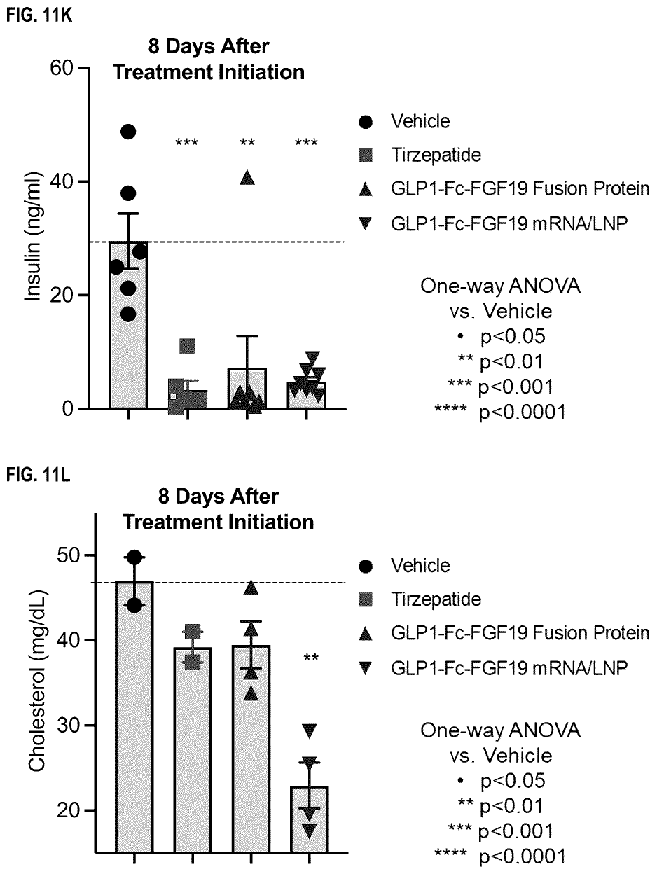

As shown in FIG. 11A, the GLP1-Fc-FGF19 fusion protein and mRNA/LNP encoding the GLP1-Fc-FGF19 fusion protein induced significant weight loss (p<0.0001) in treated animals. Tirzepatide also induced significant weight loss (p<0.0001) in treated animals (FIG. 11A). Non-fasted blood glucose was measured in the animals on days 0, 1 and 7. As shown in FIG. 11B, the blood glucose levels in control mice that received only PBS remained unchanged during the course of the experiment. On the other hand, each of tirzepatide, the GLP1-Fc-FGF19 fusion protein and mRNA/LNP encoding the GLP1-Fc-FGF19 fusion protein caused a significant decrease (p<0.0001) in blood glucose (FIG. 11B) on day 1 compared to the mice receiving vehicle only. The mmRNA was found to be more effective in decreasing blood glucose, which without wishing to be bound by theory, may be because the accumulation of protein for longer duration in mice following mRNA integration into cells.

Cumulative food intake was measured between day 4 and 7, and represented as average daily food intake. For food consumption, remaining food was weighed on days 8, and subtracted from the starting food weight on day 4. This value was then normalized to the number of mice in the cage, and the number of days the mice had access to the food prior to weighing. As shown in FIG. 11C, the food intake of mice that received the GLP1-Fc-FGF19 fusion protein and mRNA/LNP encoding the GLP1-Fc-FGF19 fusion protein decreased compared to the mice that received vehicle only. On the other hand, the mice that received tirzepatide slightly increased compared to the mice that received vehicle only (FIG. 11C).

Plasma insulin levels were measured on Day 8 in mice treated with vehicle alone, tirzepatide, the GLP-1-Fc-FGF19 fusion protein, and the mmRNA encoding the GLP-1-Fc-FGF19 fusion protein. As shown in FIG. 11K, compared to vehicle alone-treated mice, plasma insulin levels significantly decreased in mice treated with tirzepatide (p<0.001), the GLP-1-Fc-FGF19 fusion protein (p<0.01), and the mmRNA encoding the GLP-1-Fc-FGF19 fusion protein (p<0.001). Serum cholesterol levels were measured on Day 8 in mice treated with vehicle alone, tirzepatide, the GLP-1-Fc-FGF19 fusion protein, and the mmRNA encoding the GLP-1-Fc-FGF19 fusion protein. As shown in FIG. 11L, compared to vehicle alone-treated mice, serum cholesterol levels decreased in mice treated with tirzepatide (statistically not significant), the GLP-1-Fc-FGF19 fusion protein (statistically not significant), and the mmRNA encoding the GLP-1-Fc-FGF19 fusion protein (statistically significant, p<0.01). The mmRNA was found to be more effective in decreasing steady-state insulin levels and decreasing cholesterol levels, which without wishing to be bound by theory, may be because the accumulation of protein for longer duration in mice following mRNA integration into cells.

These results indicate, inter alia, that the GLP1-Fc-FGF19 fusion protein and the mmRNA encoding the GLP1-Fc-FGF19 fusion protein is effective in controlling body weight, plasma insulin, food intake, blood glucose, blood insulin and serum cholesterol.

On day 19 during terminal necropsy, liver and subcutaneous white adipose tissue were recovered. The epididymal white adipose tissue (eWAT) recovered from the mice was weighed. As shown in FIG. 11E, compared to the mice receiving vehicle only, a significant decrease in the weights of eWAT was observed in mice treated with tirzepatide (p<0.01), the GLP1-Fc-FGF19 fusion protein (p<0.01), or mRNA/LNP encoding the GLP1-Fc-FGF19 fusion protein (p<0.01). The subcutaneous white adipose tissue (sWAT) recovered from the mice was weighed. As shown in FIG. 11F, compared to the mice receiving vehicle only, a decrease in the weights of sWAT was observed in mice treated with tirzepatide (statistically insignificant), the GLP1-Fc-FGF19 fusion protein (p<0.05), or mRNA/LNP encoding the GLP1-Fc-FGF19 fusion protein (p<0.0001). The mmRNA was found to be more effective in decreasing weights of sWAT, which without wishing to be bound by theory, may be because the accumulation of protein for longer duration in mice following mRNA integration into cells.

The livers of control mice that received only PBS appeared enlarged and whitish in color, indicating an increase in adiposity. By contrast, the livers of mice receiving the treatments were pinkish in color and smaller in size, showing a decrease in adiposity. The livers were weighed. As shown in FIG. 11D, compared to the mice receiving vehicle only, a significant decrease in the weights of livers was observed in mice treated with tirzepatide (p<0.0001), the GLP1-Fc-FGF19 fusion protein (p<0.001), or mRNA/LNP encoding the GLP1-Fc-FGF19 fusion protein (p<0.001).

These results indicate, inter alia, that the GLP1-Fc-FGF19 fusion protein and the mmRNA encoding the GLP1-Fc-FGF19 fusion protein is effective in controlling liver adiposity, liver weight, epididymal white adipose tissue (eWAT) and subcutaneous white adipose tissue (sWAT) weight.

To further analyze the changes in liver histopathological analysis was conducted. Briefly, the liver tissue was stained with hematoxylin and eosin (H&E) was evaluated using standard techniques. In addition to the liver samples from this experiment, liver tissue samples from the mice treated with GLP1-Fc-GLP-1-Fc-FGF21 (RGE) fusion protein, mRNA/LNP encoding the GLP1-Fc-GLP-1-Fc-FGF21 (RGE) fusion protein (Example 11) were also analyzed along with the controls included therein. FIG. 11G shows representative images. As shown in FIG. 11G, the liver tissue from vehicle-treated mice showed enlarged cells with rarefied cytoplasm and hepatocellular ballooning, which is associated with fat droplet accumulation and steatosis. The livers from mice treated with tirzepatide showed a decrease in hepatocellular ballooning and steatosis (FIG. 11G). Interestingly, livers from mice treated with the GLP1-Fc-FGF19 fusion protein, GLP1-Fc-GLP-1-Fc-FGF21 (RGE) fusion protein, mRNA/LNP encoding the GLP1-Fc-FGF19 fusion protein, and mRNA/LNP encoding the GLP1-Fc-GLP-1-Fc-FGF21 (RGE) fusion protein a decrease in hepatocellular ballooning and steatosis that was greater than the decrease observed in mice that received tirzepatide (FIG. 11G).

To assess whether the observed hepatocellular ballooning was associated with fibrosis, liver sections stained with picrosirius red (PSR) were analyzed. FIG. 11H shows representative images. As shown in FIG. 11H, the liver tissue from vehicle-treated mice showed collagen staining, indicating existence of fibrosis. The livers from mice treated with tirzepatide showed a decrease in collagen staining, indicating a decrease in fibrosis (FIG. 11G). Interestingly, livers from mice treated with the GLP1-Fc-FGF19 fusion protein, GLP1-Fc-GLP-1-Fc-FGF21 (RGE) fusion protein, mRNA/LNP encoding the GLP1-Fc-FGF19 fusion protein, and mRNA/LNP encoding the GLP1-Fc-GLP-1-Fc-FGF21 (RGE) fusion protein a decrease in ease in collagen staining, indicating a decrease in fibrosis that was greater than the decrease observed in mice that received tirzepatide (FIG. 11H).

These results indicate, inter alia, that the GLP1-Fc-FGF19 fusion protein, the GLP-1-Fc-FGF21 (RGE) fusion protein, the mmRNA encoding the GLP1-Fc-FGF19 fusion protein, and the mmRNA encoding the GLP-1-Fc-FGF21 (RGE) is effective in decreasing hepatocellular ballooning liver, steatosis, and fibrosis.

The levels of liver glut4 mRNA were measured in the liver samples. As shown in FIG. 11I, compared to the vehicle-alone treated mice, the liver glut4 mRNA levels decreased in mice treated with tirzepatide (not statistically significant), the GLP-1-Fc-FGF19 fusion protein (p<0.00001), and the mmRNA encoding the GLP-1-Fc-FGF19 fusion protein (p<0.00001). The levels of liver lipin1 mRNA were measured in the liver samples. As shown in FIG. 11J, compared to the vehicle-alone treated mice, the liver lipin1 mRNA levels increased in mice treated with tirzepatide. Interestingly, compared to the vehicle-alone treated mice, the liver lipin1 mRNA levels decreased the GLP-1-Fc-FGF19 fusion protein, and the mmRNA encoding the GLP-1-Fc-FGF19 fusion protein (p<0.05). Therefore, it is expected that the GLP-1-Fc-FGF19 fusion protein and the mmRNA encoding the GLP-1-Fc-FGF19 fusion protein is effective against hyperglycemia and insulin resistance. See Ryu et al., TORC2 Regulates Hepatic Insulin Signaling via a Mammalian Phosphatidic Acid Phosphatase, LIPIN1, Cell Metabolism 9:240-251 (2009).

### Example 13. In Vivo Control of Liver Fibrosis, Steatosis, Body Weight, Plasma Glucose, Food Intake, and Body Fat by the Purified Chimeric Proteins and Modified mRNA (mmRNA) Encoding the Chimeric Proteins Disclosed Herein

HEPG2, human liver hepatoma cells express FGF receptors. When liver cells are exposed to fatty acids (which can be recapitulated in vitro with oleic, palmitic, and other acids), they can uptake the acid, resulting in liver cytotoxicity and dysfunction.

HEK293T cells transfected with the modified mRNA encoding the GLP-1-Fc-FGF19 and GLP-1-Fc-FGF21 (RGE) chimeric proteins, transfectants were cultures, and culture supernatants were obtained. The amounts of the GLP-1-Fc-FGF19 and GLP-1-Fc-FGF21 (RGE) chimeric proteins in the culture supernatants was measured using a Meso Scale Discovery (MSD) platform-based ELISA assay.

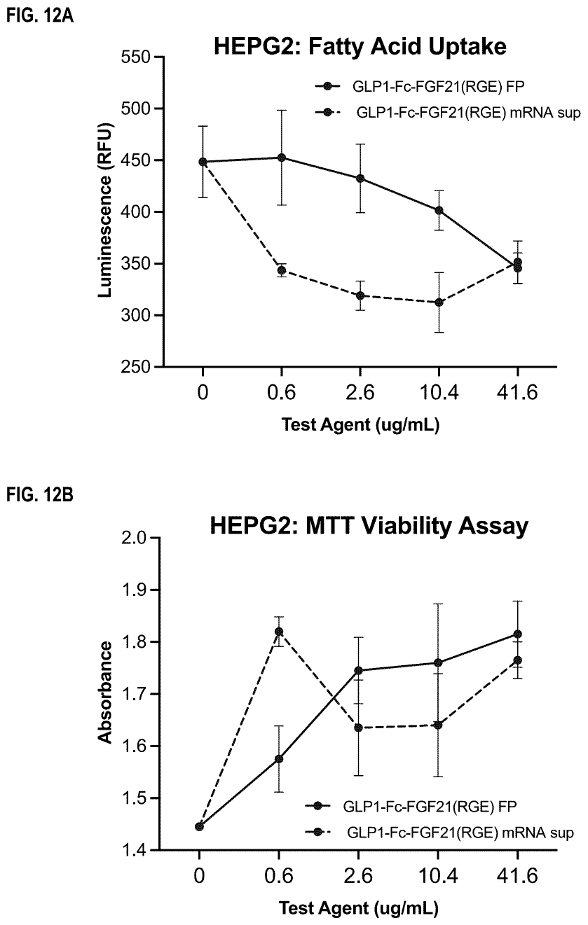

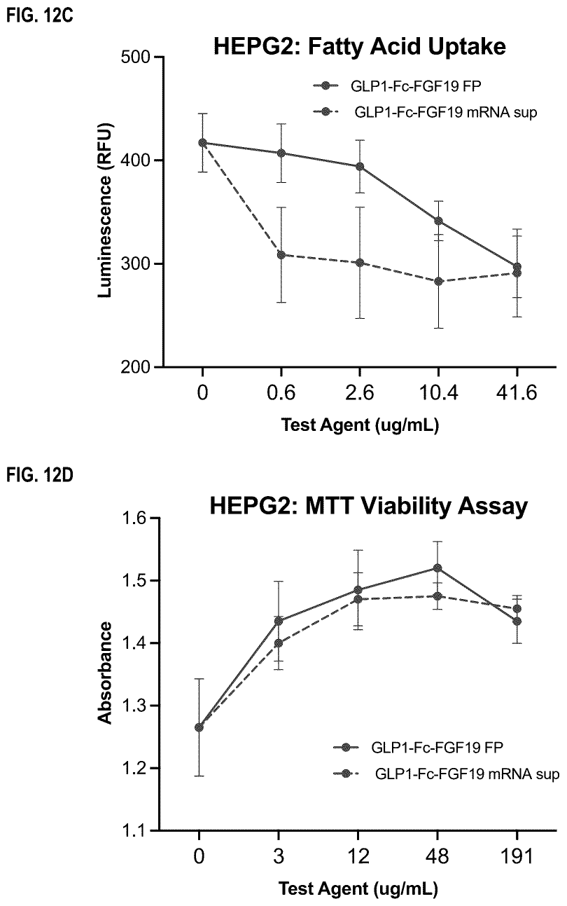

HEPG2 cells were cultured with oleic acid and fluorescently labeled oleic acid in the presence of either increasing amounts of the GLP-1-Fc-FGF21 (RGE) chimeric protein or matched concentrations of culture supernatants of HEK293T cells transfected with the modified mRNA encoding the mmRNA encoding the GLP-1-Fc-FGF21 (RGE) fusion protein. Uptake of the fluorescent label was quantitated using flow cytometry and plotted as a function of the amount of the GLP-1-Fc-FGF21 (RGE) chimeric protein. As shown in FIG. 12A, these was a dose-dependent decrease in oleic acid intake in cells treated with the GLP-1-Fc-FGF21 (RGE) chimeric protein or the culture supernatants of HEK293T cells transfected with the modified mRNA encoding the mmRNA encoding the GLP-1-Fc-FGF21 (RGE) fusion protein. These results indicate, inter alia, that the GLP-1-Fc-FGF21 (RGE) chimeric protein or the culture supernatants of HEK293T cells transfected with the modified mRNA encoding the mmRNA encoding the GLP-1-Fc-FGF21 (RGE) fusion protein inhibit fatty acid intake by liver cells.

The effect of the GLP-1-Fc-FGF21 (RGE) chimeric protein or the culture supernatants of HEK293T cells transfected with the modified mRNA encoding the mmRNA encoding the GLP-1-Fc-FGF21 (RGE) fusion protein on lethality induced by fatty acids was assessed. HEPG2 cells were cultured with oleic acid and fluorescently labeled oleic acid in the presence of either increasing amounts of the GLP-1-Fc-FGF21 (RGE) chimeric protein or matched concentrations of culture supernatants of HEK293T cells transfected with the modified mRNA encoding the mmRNA encoding the GLP-1-Fc-FGF21 (RGE) fusion protein. After 24 hours in culture, cell viability was measured using the MTT assay. As shown in FIG. 12B, the GLP-1-Fc-FGF21 (RGE) chimeric protein or the culture supernatants of HEK293T cells transfected with the modified mRNA encoding the mmRNA encoding the GLP-1-Fc-FGF21 (RGE) fusion protein induced a dose-dependent and saturable increase in viability of oleic acid HEPG2 cells were cultured with oleic acid. These results indicate, inter alia, that the GLP-1-Fc-FGF21 (RGE) chimeric protein or the culture supernatants of HEK293T cells transfected with the modified mRNA encoding the mmRNA encoding the GLP-1-Fc-FGF21 (RGE) fusion protein protect liver cells from fatty acid-induced lethality.

HEPG2 cells were cultured with oleic acid and fluorescently labeled oleic acid in the presence of either increasing amounts of the GLP-1-Fc-FGF19 chimeric protein or matched concentrations of culture supernatants of HEK293T cells transfected with the modified mRNA encoding the mmRNA encoding the GLP-1-Fc-FGF19 fusion protein. Uptake of the fluorescent label was quantitated using flow cytometry and plotted as a function of the amount of the GLP-1-Fc-FGF19 chimeric protein. As shown in FIG. 12C, these was a dose-dependent decrease in oleic acid intake in cells treated with the GLP-1-Fc-FGF19 chimeric protein or the culture supernatants of HEK293T cells transfected with the modified mRNA encoding the mmRNA encoding the GLP-1-Fc-FGF19 fusion protein. These results indicate, inter alia, that the GLP-1-Fc-FGF19 chimeric protein or the culture supernatants of HEK293T cells transfected with the modified mRNA encoding the mmRNA encoding the GLP-1-Fc-FGF19 fusion protein inhibit fatty acid intake by liver cells.

The effect of the GLP-1-Fc-FGF19 chimeric protein or the culture supernatants of HEK293T cells transfected with the modified mRNA encoding the mmRNA encoding the GLP-1-Fc-FGF19 fusion protein on lethality induced by fatty acids was assessed. HEPG2 cells were cultured with oleic acid and fluorescently labeled oleic acid in the presence of either increasing amounts of the GLP-1-Fc-FGF19 chimeric protein or matched concentrations of culture supernatants of HEK293T cells transfected with the modified mRNA encoding the mmRNA encoding the GLP-1-Fc-FGF19 fusion protein. After 24 hours in culture, cell viability was measured using the MTT assay. As shown in FIG. 12D, the GLP-1-Fc-FGF19 chimeric protein or the culture supernatants of HEK293T cells transfected with the modified mRNA encoding the mmRNA encoding the GLP-1-Fc-FGF19 fusion protein induced a dose-dependent and saturable increase in viability of oleic acid HEPG2 cells were cultured with oleic acid. These results indicate, inter alia, that the GLP-1-Fc-FGF19 chimeric protein or the culture supernatants of HEK293T cells transfected with the modified mRNA encoding the mmRNA encoding the GLP-1-Fc-FGF19 fusion protein protect liver cells from fatty acid-induced lethality.

These results indicate, inter alia, that the GLP-1-Fc-FGF19 and GLP-1-Fc-FGF21 (RGE) chimeric proteins and the modified mRNA encoding the GLP-1-Fc-FGF19 and GLP-1-Fc-FGF21 (RGE) chimeric proteins inhibit excessive fatty acid intake by liver cells and protect them from fatty acid-induced lethality.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A chimeric protein having a general structure of: 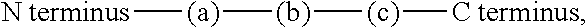
wherein:
(A)
(a) is a first domain comprising a glucagon-like peptide-1 (GLP-1) receptor agonist,
(b) is a linker adjoining the first domain and a second domain, optionally wherein the linker comprises one or more protease-cleavable polypeptide linkers, and/or a hinge-CH2-CH3 Fc domain, and
(c) is the second domain comprising a fibroblast growth factor selected from fibroblast growth factor 19 (FGF19), FGF21, FGF23, a variant thereof, and an analog thereof; or
(B)
(a) is a first domain comprising a fibroblast growth factor selected from fibroblast growth factor 19 (FGF19), FGF21, FGF23, a variant thereof, and an analog thereof,
(b) is a linker adjoining the first domain and a second domain, optionally wherein the linker comprises one or more protease-cleavable polypeptide linkers, and/or a hinge-CH2-CH3 Fc domain, and
(c) is the second domain comprising a glucagon-like peptide-1 (GLP-1) receptor agonist.

2. The chimeric protein of claim 1, wherein the GLP-1 receptor agonist is selected from GLP-1, DPP4 degradation resistant GLP-1 (7-37, A8G), exenatide, lixisenatide, albiglutide, dulaglutide, or a variant thereof having one or more amino acid mutations, independently selected from substitutions, insertions, deletions, and truncations.

3. The chimeric protein of claim 1, wherein the GLP-1 receptor agonist has an amino acid sequence of any one of SEQ ID NOs: 57 to 62, 75, and 87-90, or a variant having about 1, 2, 3, 4, 5, 6, 7, 8, 9, or 10 or more amino acid mutations with respect to an amino acid sequence selected from SEQ ID NOs: 57 to 62, 75, and 87-90.

4. The chimeric protein of any one of claims 1 to 3, wherein the GLP-1 receptor agonist is capable of binding a GLP-1 receptor.

5. The chimeric protein of any one of claims 1 to 4, wherein the GLP-1 receptor agonist is capable of stimulating insulin secretion and/or inhibiting glucagon secretion.

6. The chimeric protein of any one of claims 1 to 5, wherein the fibroblast growth factor comprises FGF19, or an analog thereof.

7. The chimeric protein of claim 6, wherein the analog of FGF19 is aldafermin (NGM282).

8. The chimeric protein of claim 6 or claim 7, wherein the fibroblast growth factor is capable of activating FGFR4, optionally wherein the activating requires β-Klotho as a coreceptor.

9. The chimeric protein of any one of claims 6 to 8, wherein the fibroblast growth factor comprises an amino acid sequence that is at least about 90%, or at least about 95%, or at least about 97%, or at least about 98%, or at least about 99% identical to an amino acid sequence of SEQ ID NOs: 65 or 66.

10. The chimeric protein of any one of claims 1 to 5, wherein the fibroblast growth factor comprises FGF21, or an analog thereof.

11. The chimeric protein of claim 10, wherein the analog of FGF21 is selected from efruxifermin, LY2405319, FGF21 (RGE) and FGF21 (L146P).

12. The chimeric protein of claim 11, wherein the fibroblast growth factor is capable of activating FGFR1c, optionally wherein the activating requires β-Klotho as a coreceptor.

13. The chimeric protein of any one of claim 6, 11 or 12, wherein the fibroblast growth factor comprises an amino acid sequence that is at least about 90%, or at least about 95%, or at least about 97%, or at least about 98%, or at least about 99% identical to an amino acid sequence selected from SEQ ID NOs: 66 to 68, 76 and 77.

14. The chimeric protein of any one of claims 1 to 13, wherein the linker comprises a protease-cleavable polypeptide linker.

15. The chimeric protein of any one of claims 1 to 14, wherein the linker comprises a protease-cleavable polypeptide linker cleavable by a protease that is endogenous to a mammalian liver, optionally, the protease being selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin.

16. The chimeric protein of any one of claims 1 to 15, wherein the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

17. The chimeric protein of claim 16, wherein the protease-cleavable polypeptide linker is C terminal to the first domain or N terminal to the second domain.

18. The chimeric protein of claim 17, wherein:
the first domain comprising a glucagon-like peptide-1 (GLP-1) receptor agonist and the protease-cleavable polypeptide linker is C terminal to the first domain; or
the second domain comprises a glucagon-like peptide-1 (GLP-1) receptor agonist and the protease-cleavable polypeptide linker is N terminal to the second domain.

19. The chimeric protein of claim 18, wherein the chimeric protein comprises two protease-cleavable polypeptide linkers, optionally wherein the protease-cleavable polypeptide linkers are independently selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74, optionally wherein the first protease-cleavable polypeptide linker is C terminal to the first domain and the second protease-cleavable polypeptide linker is N terminal to the second domain.

20. The chimeric protein of any one of claims 1 to 19, wherein the linker comprises a hinge-CH2-CH3 Fc domain derived from IgG1.

21. The chimeric protein of claim 20, wherein the IgG1 is human IgG1.

22. The chimeric protein of any one of claims 1 to 19, wherein the linker comprises a hinge-CH2-CH3 Fc domain derived from IgG4.

23. The chimeric protein of claim 22, wherein the IgG4 is human IgG4.

24. The chimeric protein of claim 23, wherein the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 79.

25. The chimeric protein of claim 24, wherein the linker further comprises the linker comprises one or more joining linkers, such joining linkers independently selected from SEQ ID NOs: 4 to 50 or 79.

26. The chimeric protein of claim 25, wherein the linker comprises two or more joining linkers each joining linker independently selected from SEQ ID NOs: 4 to 50 or 79; wherein one joining linker is N terminal to the hinge-CH2-CH3-Fc domain and another joining linker is C terminal to the hinge-CH2-CH3-Fc domain.

27. An isolated polynucleotide encoding the chimeric protein of any one of claims 1 to 26.

28. The isolated polynucleotide of claim 27, wherein the polynucleotide is mRNA or a modified mRNA (mmRNA).

29. The isolated polynucleotide of claim 28, wherein the polynucleotide is an mmRNA.

30. The isolated polynucleotide of claim 29, wherein the mmRNA comprises one or more nucleoside modifications.

31. The isolated polynucleotide of claim 30, wherein the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

32. The isolated polynucleotide of claim 31, wherein the mmRNA further comprises a 5′-cap and/or a poly A tail.

33. The isolated polynucleotide of claim 27, wherein the polynucleotide is DNA.

34. The isolated polynucleotide of claim 33, wherein the polynucleotide comprises a liver-specific control element.

35. The isolated polynucleotide of claim 34, wherein the liver-specific control element is a liver-specific promoter selected from albumin promoter, thyroxine-binding globulin (TBG) promoter, hybrid liver-specific promoter (HLP), human α1-antitrypsin promoter, LP1 promoter, and hemopexin promoter.

36. A vector comprising the polynucleotide of any one of claims 33 to 35.

37. A host cell comprising the vector of claim 36 or the mmRNA of claim 29.

38. A pharmaceutical composition comprising the chimeric protein of any one of claims 1 to 26, the isolated polynucleotide of any one of claims 27 to 35, or the vector of claim 36, the host cell of claim 37, and a pharmaceutically acceptable carrier.

39. The pharmaceutical composition of claim 38, wherein the pharmaceutical composition comprises the mmRNA of any one of claims 28 to 32.

40. The pharmaceutical composition of claim 38 or claim 39, wherein the carrier is a lipidoid, a liposome, a lipoplex, a lipid nanoparticle, a polymeric nanoparticle, a peptide, a protein, a cell, a nanoparticle mimic, a nanotube, or a conjugate.

41. The pharmaceutical composition of claim 40, wherein the pharmaceutical composition is formulated as lipid nanoparticles (LNPs), a lipoplex, or a liposome.

42. The pharmaceutical composition of claim 41, wherein the pharmaceutical composition is formulated as lipid nanoparticles (LNPs).

43. The pharmaceutical composition of claim 42, wherein the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); and dioleoylphosphatidylethanolamine (DOPE).

44. The pharmaceutical composition of claim 42, wherein the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle.

45. The pharmaceutical composition of claim 42 or claim 44, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

46. The pharmaceutical composition of any one of claims 38 to 45, wherein the pharmaceutical composition is formulated for parenteral administration.

47. The pharmaceutical composition of claim 46, wherein the pharmaceutical composition is formulated for intradermal, intramuscular, intraperitoneal, intraarticular, intravenous, subcutaneous, intraarterial or transdermal administration.

48. A method of treating or preventing nonalcoholic fatty liver disease (NAFLD), non-alcoholic steatohepatitis (NASH), obesity, or metabolic syndrome, or of reducing blood glucose, or of reducing fed and fasting blood glucose, or of reducing steady state plasma insulin, or of reducing food intake, or of reducing serum cholesterol, or of reducing liver adiposity, or of reducing liver weight, or of reducing epididymal white adipose tissue (eWAT) accumulation, or of reducing subcutaneous white adipose tissue (sWAT) accumulation, or of decreasing hepatocellular ballooning, or of reducing liver fibrosis, or of reducing excessive fatty acid intake by liver cells, or of protecting liver cells from fatty acid-induced toxicity a subject in need thereof, the method comprising administering to the subject a chimeric protein of any one of claims 1 to 26, the isolated polynucleotide of any one of claims 27 to 35, the vector of claim 36, or the host cell of claim 37, or the pharmaceutical composition of any one of claims 38 to 47.

49. A method of treating nonalcoholic fatty liver disease (NAFLD) or non-alcoholic steatohepatitis (NASH) in a subject in need thereof, the method comprising administering pharmaceutical composition comprising a polynucleotide encoding a chimeric protein having a general structure of: 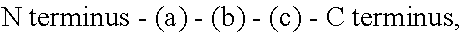
wherein:
(A)
(a) is a first domain comprising a glucagon-like peptide-1 (GLP-1) receptor agonist,
(b) is a linker adjoining the first domain and a second domain, optionally wherein the linker comprises one or more protease-cleavable polypeptide linkers, and/or a hinge-CH2-CH3 Fc domain, and
(c) is the second domain comprising a fibroblast growth factor selected from fibroblast growth factor 19 (FGF19), FGF21, FGF23, a variant thereof, and an analog thereof; or
(B)
(a) is a first domain comprising a fibroblast growth factor selected from fibroblast growth factor 19 (FGF19), FGF21, FGF23, a variant thereof, and an analog thereof,
(b) is a linker adjoining the first domain and a second domain, optionally wherein the linker comprises one or more protease-cleavable polypeptide linkers, and/or a hinge-CH2-CH3 Fc domain, and
(c) is the second domain comprising a glucagon-like peptide-1 (GLP-1) receptor agonist.

50. The method of claim 49, wherein the GLP-1 receptor agonist is selected from wherein the GLP-1 receptor agonist is selected from GLP-1, DPP4 degradation resistant GLP-1 (7-37, A8G), exenatide, lixisenatide, albiglutide, dulaglutide, or a variant thereof having one or more amino acid mutations, independently selected from substitutions, insertions, deletions, and truncations.

51. The method of claim 49 or claim 50, wherein the GLP-1 receptor agonist has an amino acid sequence of any one of SEQ ID NOs: 57 to 62, 75, and 87-90, or a variant having about 1, 2, 3, 4, 5, 6, 7, 8, 9, or 10 or more amino acid mutations with respect to an amino acid sequence selected from SEQ ID NOs: 57 to 62, 75, and 87-90.

52. The method of any one of claims 49 to 51, wherein the GLP-1 receptor agonist is capable of binding a GLP-1 receptor.

53. The method of any one of claims 49 to 52, wherein the GLP-1 receptor agonist is capable of stimulating insulin secretion and/or inhibiting glucagon secretion.

54. The method of any one of claims 49 to 53, wherein the fibroblast growth factor comprises FGF19, or an analog thereof.

55. The method of claim 54, wherein the analog of FGF19 is aldafermin (NGM282).

56. The method of claim 49, wherein the fibroblast growth factor is capable of activating FGFR4, optionally wherein the activating requires β-Klotho as a coreceptor.

57. The method of any one of claims 49 to 56, wherein the fibroblast growth factor comprises an amino acid sequence that is at least about 90%, or at least about 95%, or at least about 97%, or at least about 98%, or at least about 99% identical to an amino acid sequence selected from SEQ ID NOs: 65 or 66.

58. The method of any one of claims 49 to 53, wherein the fibroblast growth factor comprises FGF21, or an analog thereof.

59. The method of claim 58, wherein the analog of FGF21 is selected from efruxifermin, LY2405319, FGF21 (RGE) and FGF21 (L146P).

60. The method of claim 58 or claim 59, wherein the fibroblast growth factor is capable of activating FGFR1c, optionally wherein the activating requires β-Klotho as a coreceptor.

61. The method of any one of claims 49 to 53, or 58 to 60, wherein the fibroblast growth factor comprises an amino acid sequence that is at least about 90%, or at least about 95%, or at least about 97%, or at least about 98%, or at least about 99% identical to an amino acid sequence selected from SEQ ID NOs: 66 to 68, 76 and 77.

62. The method of any one of claims 49 to 61, wherein the linker comprises a protease-cleavable polypeptide linker.

63. The method of any one of claims 49 to 62, wherein the linker comprises a protease-cleavable polypeptide linker cleavable by a protease that is endogenous to a mammalian liver, optionally, the protease being selected from, caspases, kallikreins, cathepsins, legumain, matrix metalloproteinases (MMPs), cathepsin, elastase, plasmin, thrombin, trypsin, urokinase-type plasminogen activator (uPA), matriptase, meprins and hepsin

64. The method of any one of claims 49 to 63, wherein the chimeric protein comprises one protease-cleavable polypeptide linker selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74.

65. The method of any one of claims 49 to 64, wherein the protease-cleavable polypeptide linker is C terminal to the first domain or N terminal to the second domain.

66. The method of claim 65, wherein:
the first domain comprising a glucagon-like peptide-1 (GLP-1) receptor agonist and the protease-cleavable polypeptide linker is C terminal to the first domain; or
the second domain comprises a glucagon-like peptide-1 (GLP-1) receptor agonist and the protease-cleavable polypeptide linker is N terminal to the second domain.

67. The method of any one of claims 49 to 66, wherein the chimeric protein comprises two protease-cleavable polypeptide linkers, optionally wherein the protease-cleavable polypeptide linkers are independently selected from HSSKLQ (SEQ ID NO: 70), GPLGVRG (SEQ ID NO: 71), IPVSLRSG (SEQ ID NO: 72), VPLSLYSG (SEQ ID NO: 73), SGESPAYYTA (SEQ ID NO: 74), and RFRS (SEQ ID NO: 78), or a variant thereof having about 1, 2, 3, 4, or more amino acid mutations with respect to an amino acid sequence selected from SEQ ID NOs: 70 to 74, optionally wherein the first protease-cleavable polypeptide linker is C terminal to the first domain and the second protease-cleavable polypeptide linker is N terminal to the second domain.

68. The method of any one of claims 49 to 67, wherein the linker comprises a hinge-CH2-CH3 Fc domain derived from IgG1.

69. The method of claim 68, wherein the IgG1 is human IgG1.

70. The method of any one of claims 49 to 65, wherein the linker comprises a hinge-CH2-CH3 Fc domain derived from IgG4.

71. The method of claim 68, wherein the IgG4 is human IgG4.

72. The method of claim 71, wherein the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 79.

73. The method of claim 72, wherein the linker further comprises the linker comprises one or more joining linkers, such joining linkers independently selected from SEQ ID NOs: 4 to 50 or 79.

74. The method of claim 73, wherein the linker comprises two or more joining linkers each joining linker independently selected from SEQ ID NOs: 4 to 50 or 79; wherein one joining linker is N terminal to the hinge-CH2-CH3-Fc domain and another joining linker is C terminal to the hinge-CH2-CH3-Fc domain.

75. The method of any one of claims 49 to 74, wherein the polynucleotide is an mmRNA.

76. The method of claim 75, wherein the mmRNA comprises one or more nucleoside modifications.

77. The method of claim 76, wherein the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

78. The method of claim 77, wherein the mmRNA further comprises a 5′-cap and/or a poly A tail.

79. The method of any one of claims 49 to 78, wherein the pharmaceutical composition further comprises a carrier.

80. The method of claim 79, wherein the carrier is a lipidoid, a liposome, a lipoplex, a lipid nanoparticle, a polymeric nanoparticle, a peptide, a protein, a cell, a nanoparticle mimic, a nanotube, or a conjugate.

81. The method of any one of claims 49 to 78, wherein the polynucleotide is formulated as lipid nanoparticles (LNPs), a lipoplex, or a liposome.

82. The method of claim 81, wherein the polynucleotide is formulated as lipid nanoparticles (LNPs).

83. The method of claim 82, wherein the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18); 1,2-dioleoyl-3-trimethylammoniumpropane (DOTAP); and dioleoylphosphatidylethanolamine (DOPE).

84. The method of claim 83, wherein the lipid nanoparticles comprise an ionizable lipid, a PEG-lipid, a phospholipid and a structural lipid.

85. The method of claim 83 or claim 84, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

86. The method of any one of claims 81 to 85, wherein the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle, and/or wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

87. The method of any one of claims 49 to 86, wherein the isolated polynucleotide administered by a parenteral administration.

88. The method of claim 87, wherein the parenteral administration is selected from intradermal, intramuscular, intraperitoneal, intraarticular, intravenous, subcutaneous, intraarterial or transdermal administration.

89. The method of any one of claims 49 to 88, wherein the chimeric protein is expressed in liver.

90. The method of any one of claims 49 to 89, wherein the chimeric protein is cleaved in liver.

91. The method of any one of claims 49 to 88, wherein the GLP-1 receptor agonist is released in liver.

92. The method of claim 91, wherein the GLP-1 receptor agonist enters circulation upon release in liver.

93. The method of any one of claims 49 to 92, wherein the subject is obese or at risk of obesity.

94. The method of any one of claims 49 to 93, wherein the subject is suffering from metabolic syndrome.

95. The method of any one of claims 49 to 94, wherein the subject has high blood glucose.

96. The method of claim 49, wherein the subject has high fed and fasting blood glucose.

97. A method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: 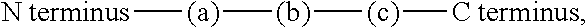
wherein:
(A)
(a) is a first domain comprising glucagon-like peptide-1 (GLP-1),
(b) is a linker adjoining the first domain and a second domain, optionally wherein the linker comprises one or more protease-cleavable polypeptide linkers, and/or a hinge-CH2-CH3 Fc domain, and
(c) is the second domain comprising a fibroblast growth factor 19 (FGF19); or
(B)
(a) is a first domain comprising a fibroblast growth factor 19 (FGF19),
(b) is a linker adjoining the first domain and a second domain, optionally wherein the linker comprises one or more protease-cleavable polypeptide linkers, and/or a hinge-CH2-CH3 Fc domain, and
(c) is the second domain comprising glucagon-like peptide-1 (GLP-1).

98. A method of treating nonalcoholic fatty liver disease (NAFLD) or non-alcoholic steatohepatitis (NASH), in a subject in need thereof, the method comprising administering a pharmaceutical composition comprising a modified mRNA (mmRNA) encoding a chimeric protein having a general structure of: 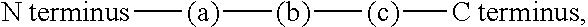
wherein:
(A)
(a) is a first domain comprising glucagon-like peptide-1 (GLP-1),
(b) is a linker adjoining the first domain and a second domain, optionally wherein the linker comprises one or more protease-cleavable polypeptide linkers, and/or a hinge-CH2-CH3 Fc domain, and
(c) is the second domain comprising a fibroblast growth factor 21 (FGF21); or
(B)
(a) is a first domain comprising a fibroblast growth factor 21 (FGF21),
(b) is a linker adjoining the first domain and a second domain, optionally wherein the linker comprises one or more protease-cleavable polypeptide linkers, and/or a hinge-CH2-CH3 Fc domain, and
(c) is the second domain comprising glucagon-like peptide-1 (GLP-1).

99. The method of claim 97 or claim 98, wherein the mmRNA comprises one or more nucleoside modifications.

100. The method of any one of claims 97 to 99, wherein the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 2-aminoadenine, 7-deaza-adenine, 7-deaza-8-aza-adenine, 7-deaza-2-aminopurine, 7-deaza-8-aza-2-aminopurine, 7-deaza-2,6-diaminopurine, 6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

101. The method of claim 100, wherein the mmRNA further comprises a 5′-cap and/or a poly A tail.

102. The method of any one of claims 97 to 101, wherein the pharmaceutical composition further comprises a carrier.

103. The method of claim 102, wherein the carrier is a lipidoid, a liposome, a lipoplex, a lipid nanoparticle, a polymeric nanoparticle, a peptide, a protein, a cell, a nanoparticle mimic, a nanotube, or a conjugate.

104. The method of any one of claims 97 to 102, wherein the polynucleotide is formulated as lipid nanoparticles (LNPs), a lipoplex, or a liposome.

105. The method of any one of claims 97 to 104, wherein the polynucleotide is formulated as lipid nanoparticles (LNPs).

106. The method of claim 105, wherein the lipid nanoparticles comprise lipids selected from one or more of an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); and dioleoylphosphatidylethanolamine (DOPE).

107. The method of claim 106, wherein the lipid nanoparticles comprise an ionizable lipid, a PEG-lipid, a phospholipid and a structural lipid.

108. The method of claim 106 or claim 107, wherein the lipid nanoparticles comprise (a) a cationic lipid comprising from about 50 mol % to about 85 mol % of the total lipid present in the particle; (b) a non-cationic lipid comprising from about 13 mol % to about 49.5 mol % of the total lipid present in the particle; and (c) a conjugated lipid that inhibits aggregation of particles comprising from about 0.5 mol % to about 2 mol % of the total lipid present in the particle.

109. The method of any one of claims 103 to 108, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

110. The method of any one of claims 97 to 109, wherein the isolated polynucleotide administered by a parenteral administration.

111. The method of claim 110, wherein the parenteral administration is selected from intradermal, intramuscular, intraperitoneal, intraarticular, intravenous, subcutaneous, intraarterial or transdermal administration.

112. The method of any one of claims 97 to 111, wherein the chimeric protein is expressed in liver.

113. The method of any one of claims 97 to 112, wherein the chimeric protein is cleaved in liver.

114. The method of any one of claims 97 to 113, wherein the GLP-1 receptor agonist is released in liver.

115. The method of any one of claims 97 to 114, wherein the GLP-1 receptor agonist enters circulation upon release in liver.

116. The method of any one of claims 48 to 115, wherein the treatment of NAFLD comprises a reduction in hepatic steatosis.

117. The method of any one of claims 48 to 116, wherein the treatment of NAFLD comprises a reduction in hepatic inflammation.

118. The method of any one of claims 48 to 117, wherein the NAFLD activity score (NAS) following administration is 7 or less.

119. The method of any one of claims 48 to 118, wherein the NAS following administration is 5 or less.

120. The method of any one of claims 48 to 119, wherein the NAS following administration is 3 or less.

121. The method of any one of claims 48 to 120, wherein the subject has hepatic cirrhosis associated with the NAFLD.

122. The method of any one of claims 48 to 121, wherein the subject has hepatic cirrhosis as a comorbidity.

123. The method of any one of claims 48 to 122, wherein the subject has hepatic cirrhosis caused by the NAFLD.

124. The method of any one of claims 48 to 123, wherein the NAFLD is NAFL with attendant liver cirrhosis.

125. The method of any one of claims 48 to 124, wherein the treatment of the NAFLD comprises treatment of liver cirrhosis.

126. The method of any one of claims 48 to 125, wherein the treatment of NAFLD decreases the level of serum bile acids in the subject.

127. The method of any one of claims 48 to 126, wherein the treatment of NAFLD comprises treatment of pruritus.

128. The method of any one of claims 48 to 127, wherein the NAFLD is simple nonalcoholic fatty liver (NAFL).

129. The method of claim 128, wherein the treatment of NAFL comprises treatment of pruritus.

130. The method of claim 128, wherein the treatment of NAFL decreases the level of serum bile acids in the subject.

131. The method of any one of claims 48 to 123 or 125 to 127, wherein the NAFLD is nonalcoholic steatohepatitis (NASH).

132. The method of any one of claim 48 to 123, 125 to 127, or 131, wherein the NAFLD is NASH with attendant liver cirrhosis.

133. The method of any one of claims 48 to 123, 125 to 127, or 131 to 132, wherein the treatment of NASH decreases the level of serum bile acids in the subject.

134. The method of any one of claims 48 to 123, 125 to 127, or 131 to 133, wherein the treatment of NASH comprises treatment of pruritus.


SEQUENCE LISTING (94 sequences)

SEQ ID NO: 1 - protein, 217 aa, synthetic construct
  REGION 1..217: note = Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, synthetic construct
  REGION 1..217: note = Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, synthetic construct
  REGION 1..217: note = Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, synthetic construct
  REGION 1..11: note = Synthetic Polypeptide
SKYGPPCPSCP

SEQ ID NO: 5 - protein, 11 aa, synthetic construct
  REGION 1..11: note = Synthetic Polypeptide
SKYGPPCPPCP

SEQ ID NO: 6 - protein, 6 aa, synthetic construct
  REGION 1..6: note = Synthetic Polypeptide
SKYGPP

SEQ ID NO: 7 - protein, 6 aa, synthetic construct
  REGION 1..6: note = Synthetic Polypeptide
IEGRMD

SEQ ID NO: 8 - protein, 9 aa, synthetic construct
  REGION 1..9: note = Synthetic Polypeptide
GGGVPRDCG

SEQ ID NO: 9 - protein, 15 aa, synthetic construct
  REGION 1..15: note = Synthetic Polypeptide
IEGRMDGGGGAGGGG

SEQ ID NO: 10 - protein, 8 aa, synthetic construct
  REGION 1..8: note = Synthetic Polypeptide
GGGSGGGS

SEQ ID NO: 11 - protein, 12 aa, synthetic construct
  REGION 1..12: note = Synthetic Polypeptide
GGGSGGGGSGGG

SEQ ID NO: 12 - protein, 14 aa, synthetic construct
  REGION 1..14: note = Synthetic Polypeptide
EGKSSGSGSESKST

SEQ ID NO: 13 - protein, 4 aa, synthetic construct
  REGION 1..4: note = Synthetic Polypeptide
GGSG

SEQ ID NO: 14 - protein, 12 aa, synthetic construct
  REGION 1..12: note = Synthetic Polypeptide
GGSGGGSGGGSG

SEQ ID NO: 15 - protein, 15 aa, synthetic construct
  REGION 1..15: note = Synthetic Polypeptide
EAAAKEAAAKEAAAK

SEQ ID NO: 16 - protein, 20 aa, synthetic construct
  REGION 1..20: note = Synthetic Polypeptide
EAAAREAAAREAAAREAAAR

SEQ ID NO: 17 - protein, 17 aa, synthetic construct
  REGION 1..17: note = Synthetic Polypeptide
GGGGSGGGGSGGGGSAS

SEQ ID NO: 18 - protein, 9 aa, synthetic construct
  REGION 1..9: note = Synthetic Polypeptide
GGGGAGGGG

SEQ ID NO: 19 - skipped

SEQ ID NO: 20 - protein, 6 aa, synthetic construct
  REGION 1..6: note = Synthetic Polypeptide
GSGSGS

SEQ ID NO: 21 - protein, 10 aa, synthetic construct
  REGION 1..10: note = Synthetic Polypeptide
GSGSGSGSGS

SEQ ID NO: 22 - protein, 7 aa, synthetic construct
  REGION 1..7: note = Synthetic Polypeptide
GGGGSAS

SEQ ID NO: 23 - protein, 20 aa, synthetic construct
  REGION 1..20: note = Synthetic Polypeptide
APAPAPAPAPAPAPAPAPAP

SEQ ID NO: 24 - protein, 4 aa, synthetic construct
  REGION 1..4: note = Synthetic Polypeptide
CPPC

SEQ ID NO: 25 - protein, 5 aa, synthetic construct
  REGION 1..5: note = Synthetic Polypeptide
GGGGS

SEQ ID NO: 26 - protein, 10 aa, synthetic construct
  REGION 1..10: note = Synthetic Polypeptide
GGGGSGGGGS

SEQ ID NO: 27 - protein, 15 aa, synthetic construct
  REGION 1..15: note = Synthetic Polypeptide
GGGGSGGGGSGGGGS

SEQ ID NO: 28 - protein, 20 aa, synthetic construct
  REGION 1..20: note = Synthetic Polypeptide
GGGGSGGGGSGGGGSGGGGS

SEQ ID NO: 29 - protein, 25 aa, synthetic construct
  REGION 1..25: note = Synthetic Polypeptide
GGGGSGGGGSGGGGSGGGGSGGGGS

SEQ ID NO: 30 - protein, 30 aa, synthetic construct
  REGION 1..30: note = Synthetic Polypeptide
GGGGSGGGGSGGGGSGGGGSGGGGSGGGGS

SEQ ID NO: 31 - protein, 35 aa, synthetic construct
  REGION 1..35: note = Synthetic Polypeptide
GGGGSGGGGSGGGGSGGGGSGGGGSGGGGSGGGGS

SEQ ID NO: 32 - protein, 40 aa, synthetic construct
  REGION 1..40: note = Synthetic Polypeptide
GGGGSGGGGSGGGGSGGGGSGGGGSGGGGSGGGGSGGGGS

SEQ ID NO: 33 - protein, 16 aa, synthetic construct
  REGION 1..16: note = Synthetic Polypeptide
GGSGGSGGGGSGGGGS

SEQ ID NO: 34 - protein, 8 aa, synthetic construct
  REGION 1..8: note = Synthetic Polypeptide
GGGGGGGG

SEQ ID NO: 35 - protein, 6 aa, synthetic construct
  REGION 1..6: note = Synthetic Polypeptide
GGGGGG

SEQ ID NO: 36 - protein, 5 aa, synthetic construct
  REGION 1..5: note = Synthetic Polypeptide
EAAAK

SEQ ID NO: 37 - protein, 10 aa, synthetic construct
  REGION 1..10: note = Synthetic Polypeptide
EAAAKEAAAK

SEQ ID NO: 38 - protein, 15 aa, synthetic construct
  REGION 1..15: note = Synthetic Polypeptide
EAAAKEAAAKEAAAK

SEQ ID NO: 39 - protein, 12 aa, synthetic construct
  REGION 1..12: note = Synthetic Polypeptide
AEAAAKEAAAKA

SEQ ID NO: 40 - protein, 17 aa, synthetic construct
  REGION 1..17: note = Synthetic Polypeptide
AEAAAKEAAAKEAAAKA

SEQ ID NO: 41 - protein, 22 aa, synthetic construct
  REGION 1..22: note = Synthetic Polypeptide
AEAAAKEAAAKEAAAKEAAAKA

SEQ ID NO: 42 - protein, 27 aa, synthetic construct
  REGION 1..27: note = Synthetic Polypeptide
AEAAAKEAAAKEAAAKEAAAKEAAAKA

SEQ ID NO: 43 - protein, 46 aa, synthetic construct
  REGION 1..46: note = Synthetic Polypeptide
AEAAAKEAAAKEAAAKEAAAKALEAEAAAKEAAAKEAAAKEAAAKA

SEQ ID NO: 44 - protein, 5 aa, synthetic construct
  REGION 1..5: note = Synthetic Polypeptide
PAPAP

SEQ ID NO: 45 - protein, 18 aa, synthetic construct
  REGION 1..18: note = Synthetic Polypeptide
KESGSVSSEQLAQFRSLD

SEQ ID NO: 46 - protein, 12 aa, synthetic construct
  REGION 1..12: note = Synthetic Polypeptide
GSAGSAAGSGEF

SEQ ID NO: 47 - protein, 5 aa, synthetic construct
  REGION 1..5: note = Synthetic Polypeptide
GGGSE

SEQ ID NO: 48 - protein, 5 aa, synthetic construct
  REGION 1..5: note = Synthetic Polypeptide
GSESG

SEQ ID NO: 49 - protein, 5 aa, synthetic construct
  REGION 1..5: note = Synthetic Polypeptide
GSEGS

SEQ ID NO: 50 - protein, 35 aa, synthetic construct
  REGION 1..35: note = Synthetic Polypeptide
GEGGSGEGSSGEGSSSEGGGSEGGGSEGGGSEGGS

SEQ ID NO: 51 - protein, 234 aa, synthetic construct
  REGION 1..234: note = Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, synthetic construct
  REGION 1..234: note = Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, synthetic construct
  REGION 1..234: note = Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, synthetic construct
  REGION 1..234: note = Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, synthetic construct
  REGION 1..234: note = Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, synthetic construct
  REGION 1..234: note = Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 30 aa, synthetic construct
  REGION 1..30: note = Synthetic Polypeptide
HAEGTFTSDVSSYLEGQAAKEFIAWLVKGR

SEQ ID NO: 58 - protein, 39 aa, synthetic construct
  REGION 1..39: note = Synthetic Polypeptide
HGEGTFTSDLSKQMEEEAVRLFIEWLKNGGPSSGAPPPS

SEQ ID NO: 59 - protein, 44 aa, synthetic construct
  REGION 1..44: note = Synthetic Polypeptide
HGEGTFTSDLSKQMEEEAVRLFIEWLKNGGPSSGAPPSKKKKKK

SEQ ID NO: 60 - protein, 30 aa, synthetic construct
  REGION 1..30: note = Synthetic Polypeptide
HGEGTFTSDVSSYLEGQAAKEFIAWLVKGR

SEQ ID NO: 61 - protein, 38 aa, synthetic construct
  REGION 1..38: note = Synthetic Polypeptide
HGEGTFTSDLSKQMEEEAVRLFEWLKNGGPSSGAPPPS

SEQ ID NO: 62 - protein, 31 aa, synthetic construct
  REGION 1..31: note = Synthetic Polypeptide
HGEGTFTSDVSSYLEEQAAKEFIAWLVKGGG

SEQ ID NO: 63 - protein, 275 aa, synthetic construct
  REGION 1..275: note = Synthetic Polypeptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 192 aa, synthetic construct
  REGION 1..192: note = Synthetic Polypeptide
LAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 65 - protein, 190 aa, synthetic construct
  REGION 1..190: note = Synthetic Polypeptide
MRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 66 - protein, 181 aa, synthetic construct
  REGION 1..181: note = Synthetic Polypeptide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 67 - protein, 181 aa, synthetic construct
  REGION 1..181: note = Synthetic Polypeptide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 68 - protein, 177 aa, synthetic construct
  REGION 1..177: note = Synthetic Polypeptide
DSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLAMVGPSQGRSPSYAS

SEQ ID NO: 69 - protein, 424 aa, synthetic construct
  REGION 1..424: note = Synthetic Polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 70 - protein, 6 aa, synthetic construct
  REGION 1..6: note = Synthetic Polypeptide
HSSKLQ

SEQ ID NO: 71 - protein, 7 aa, synthetic construct
  REGION 1..7: note = Synthetic Polypeptide
GPLGVRG

SEQ ID NO: 72 - protein, 8 aa, synthetic construct
  REGION 1..8: note = Synthetic Polypeptide
IPVSLRSG

SEQ ID NO: 73 - protein, 8 aa, synthetic construct
  REGION 1..8: note = Synthetic Polypeptide
VPLSLYSG

SEQ ID NO: 74 - protein, 10 aa, synthetic construct
  REGION 1..10: note = Synthetic Polypeptide
SGESPAYYTA

SEQ ID NO: 75 - protein, 31 aa, synthetic construct
HGEGTFTSDVSSYLEEQAAKEFIAWLVKGRG

SEQ ID NO: 76 - protein, 181 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 77 - protein, 181 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHPPGNKSPHRDPAPRGPARFLPLPGLPPALPEPPGILAPQPPDVGSSDPLSMVGPSQGRSPSYAS

SEQ ID NO: 78 - protein, 4 aa, synthetic construct
RFRS

SEQ ID NO: 79 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 12 aa, synthetic construct
EPKSCDKTHTCP

SEQ ID NO: 81 - protein, 459 aa, synthetic construct
HGEGTFTSDVSSYLEEQAAKEFIAWLVKGRGEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDMRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 82 - protein, 450 aa, synthetic construct
HGEGTFTSDVSSYLEEQAAKEFIAWLVKGRGEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 83 - protein, 450 aa, synthetic construct
HGEGTFTSDVSSYLEEQAAKEFIAWLVKGRGEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHPPGNKSPHRDPAPRGPARFLPLPGLPPALPEPPGILAPQPPDVGSSDPLSMVGPSQGRSPSYAS

SEQ ID NO: 84 - protein, 463 aa, synthetic construct
HGEGTFTSDVSSYLEEQAAKEFIAWLVKGRGRFRSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDMRDSSPLVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 85 - protein, 454 aa, synthetic construct
HGEGTFTSDVSSYLEEQAAKEFIAWLVKGRGRFRSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 86 - protein, 454 aa, synthetic construct
HGEGTFTSDVSSYLEEQAAKEFIAWLVKGRGRFRSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHPPGNKSPHRDPAPRGPARFLPLPGLPPALPEPPGILAPQPPDVGSSDPLSMVGPSQGRSPSYAS

SEQ ID NO: 87 - protein, 180 aa, synthetic construct
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 88 - protein, 36 aa, synthetic construct
HDEFERHAEGTFTSDVSSYLEGQAAKEFIAWLVKGR

SEQ ID NO: 89 - protein, 30 aa, synthetic construct; identical to SEQ ID NO: 57

SEQ ID NO: 90 - protein, 31 aa, synthetic construct
HAEGTFTSDVSSYLEGQAAKEFIAWLVRGRG

SEQ ID NO: 91 - DNA, 235 bp, synthetic construct
  source 1..235: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaattgccatgtgtatgtgggttcgcccacatactctgatgatccccaatcgtggcgtgtcggcctgcttcggcaggcactggcgccgggatcattcatggcaa

SEQ ID NO: 92 - DNA, 451 bp, synthetic construct
  source 1..451: mol_type = other DNA
aaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 93 - DNA, 50 bp, synthetic construct
  source 1..50: mol_type = other DNA
acatttgcttctgacacaactgtgttcactagcaacctcaaacagacacc

SEQ ID NO: 94 - DNA, 398 bp, synthetic construct
  source 1..398: mol_type = other DNA
ttgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaagggcgaattcgttggtaaagccacc